(19) United States
(12) Patent Application Publication
(10) Pub. No.: US 2026/0159829 A1
(43) Pub. Date: Jun. 11, 2026

(54) METHODS AND SYSTEMS FOR TARGET CAPTURE LONG-READ SEQUENCING WITH MULTIPLEXING

(71) Applicants: Monica Sentmanat, St. Louis, MO (US); Xiaoxia Cui, St. Louis, MO (US)

(72) Inventors: Monica Sentmanat, St. Louis, MO (US); Xiaoxia Cui, St. Louis, MO (US)

(73) Assignee: Washington University, St. Louis, MO (US)

(21) Appl. No.: 19/387,616

(22) Filed: Nov. 12, 2025

Related U.S. Application Data
(60) Provisional application No. 63/719,601, filed on Nov. 12, 2024.

(51) Int. Cl. C12N 15/10; C12N 5/073; C12N 5/074; C12N 9/22; C12N 15/11; C12N 15/86; C12N 15/90

(52) U.S. Cl. CPC C12N 15/1065; C12N 5/0604; C12N 5/0696; C12N 9/226; C12N 15/11; C12N 15/86; C12N 15/907; C12N 2310/20; C12N 2510/00; C12N 2750/14143

(57) ABSTRACT

Assays and methods for target-capture long-read sequencing, including multiplexed assays and methods, are provided. Knockin models and constructs are also provided. In exemplary embodiments, multiple AAV donors are used, including at least two rAAV donors.

### CROSS-REFERENCE TO RELATED APPLICATIONS

This application claims priority from U.S. Provisional Application Ser. No. 63/719,601 filed on Nov. 12, 2024, which is incorporated herein by reference in its entirety.

### STATEMENT OF FEDERALLY SPONSORED RESEARCH OR DEVELOPMENT

Not applicable.

### MATERIAL INCORPORATED BY REFERENCE

The Sequence Listing, which is a part of the present disclosure, includes a computer-readable form comprising nucleotide and/or amino acid sequences of the present invention (file name “020942-US-NP_2025-11-12_Sequence-Listing” created 7 Nov. 2025; 1,583,987 bytes). The subject matter of the Sequence Listing is incorporated herein by reference in its entirety.

### FIELD

The present disclosure generally relates to methods of sample preparation and long-read sequencing assays.

### BACKGROUND

Genome engineering is indispensable for both emerging medical applications, such as gene and cell therapies, and the creation of research tools, including cell lines and model organisms. The continuous advancement of programmable nucleases, especially since the advent of CRISPR technologies in 2012, enables the introduction of increasingly sophisticated genomic modifications, allowing the recapitulation or correction of disease-relevant patient mutations, delivery of precise therapeutic cargos, or generation of a wide variety types of research models (e.g., floxed alleles, reporter lines, inducible expression) to facilitate mechanistic studies. However, site-specific large insertions remain far more challenging than simpler modifications such as knockouts, deletions, and point mutations.

Editing single-cell mouse embryos is limited in embryo numbers, both because of the source of the embryos and the need of surgical implantation post manipulation. Electroporation of CRISPR RNPs and small single-stranded DNA donors into mouse embryos circumvents the need for microinjection and enables more embryos to be edited and implanted with remarkable efficiency for point mutations and small inserts, such as loxP site. Plasmid DNA donors, however, must be microinjected into individual embryos, limiting throughput. The issue was partially addressed when rAAV donors were shown to sufficiently mediate HDR via simple incubation prior to electroporation of embryos with RNPs, bypassing the need for pronuclear microinjection.

One limitation of using rAAV is its maximum 4.7 kb payload, restricting insert size. Successful large insertions are commonly identified by junction PCRs spanning from the insert into genomic sequences adjacent to homology arms. PCRs do not always work across the insertion junctions even when an integration occurs, depending on the locus and sequence context. This leads to substantial challenges with false negatives, as a failed PCR cannot be distinguished from a failed integration, and there are no obvious positive controls for these reactions. In addition, DNA templates in either plasmid or viral forms can randomly integrate into the genome, which may confound the intended use of the engineered research model.

While high fidelity, long-read, whole genome sequencing with deep coverage is ideal for thorough characterization of the edited genome, it is too costly and low-throughput as a screening tool. Targeted alternatives have been described, namely Cas9-targeted sequencing (nCATS) with or without long DNA size-selection steps, but the on-target coverage and throughput are too low for screening multiple founders and/or clones.

Overall, two bottlenecks exist for screening mice for targeted insertion of large cassettes (>500 bp) by long-read sequencing. First, low-input material is typically the starting point. The reason is that only a small amount of tissue (<2-3 mm) can be collected from live mice intended for breeding. The tissue is usually a 1-2 mm tail snip, which makes it challenging to extract high-quality, high-molecular-weight material. Yields are typically less than 1-2 ug and vary across samples. However, there are no unbiased, pre-amplification processes currently available to enable the required representation of all samples. Second, cost is prohibitory and reduced cost methods are needed.

### BRIEF DESCRIPTION OF THE DISCLOSURE

Among the various aspects of the present disclosure is the provision of multiplexed long-read sequencing.

In accordance with an aspect of the present disclosure, a method for preparing long-read sequence samples for multiplexing is provided. The method comprising: providing a genomic DNA sample; barcoding the genomic DNA sample with an adapted Tn5 barcode to generate a barcoded material; fragmenting the barcoded material, wherein the fragmented barcoded material is longer than about 2 kb; and amplifying the fragmented barcoded material prior to multiplexing.

In some embodiments, the genomic DNA sample is a low-input genomic DNA sample, such as comprising at least about 250 ng and not more than about 5 μg. In some embodiments, the adapted Tn5 barcode is pre-loaded with unique universal flanking sequences, such as comprising Illumina P5 and P7. In some embodiments, the fragmenting is performed at a temperature of about 37° C., with a buffer comprising PEG, TAPS, and MgCl₂, and/or comprises an incubation time of at least about 15 minutes and not more than about 20 minutes. In some embodiments, the barcoding and fragmenting steps are performed simultaneously in a tagmentation step. In some embodiments, the amplifying is performed in two rounds of PCR.

In accordance with another aspect of the present disclosure, a method for generating a knockin (KI) model is provided. The method comprising: providing at least two rAAV donor vectors; and inserting the more than one rAAV donor vectors with CRISPR/Cas9 to generate the KI model.

In some embodiments, the KI model is a multi-kilobase KI model. In some embodiments, providing at least two rAAV donor vectors comprises providing at least three rAAV donor vectors. In some embodiments, the KI model is a mouse embryo KI model. In some embodiments, the KI model is an iPSC cell line KI model.

In accordance with a further aspect of the present disclosure, a knockin (KI) model is provided. The KI model having been synthesized according to the method comprising: providing at least two rAAV donor vectors; and inserting the more than one rAAV donor vectors with CRISPR/Cas9 to generate the KI model.

In some embodiments, the KI model is a multi-kilobase KI model. In some embodiments, providing at least two rAAV donor vectors comprises providing at least three rAAV donor vectors. In some embodiments, the KI model is a mouse embryo KI model. In some embodiments, the KI model is an iPSC cell line KI model.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings described herein are for illustrative purposes only. The drawings are not intended to limit the scope of the present teachings in any way.

FIG. 1 is a schematic describing long-read sequencing methods of the present disclosure.

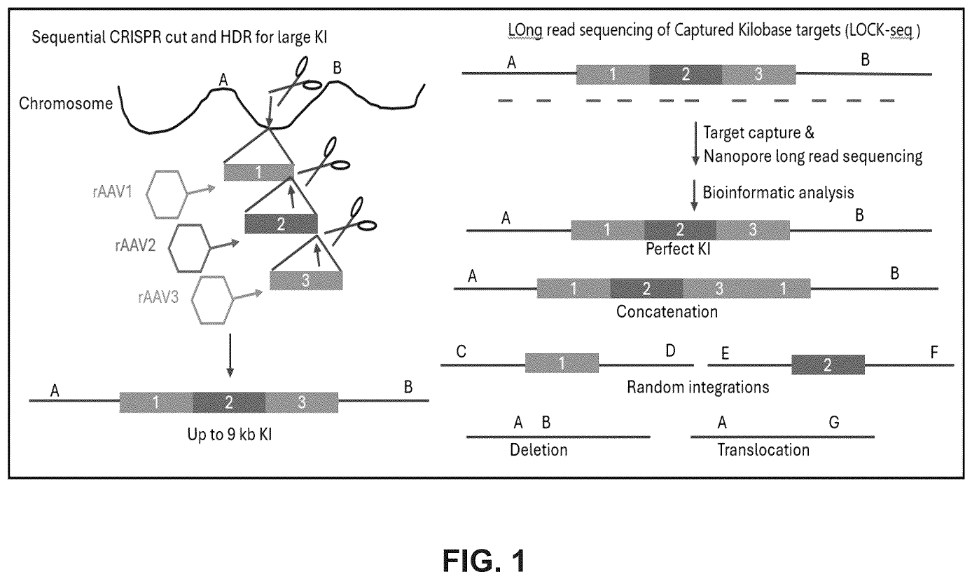

FIG. 2A is a schematic of rAAV donor and junction PCR designs.

FIG. 2B is a scatter plot of KI efficiency by junction PCRs vs. insert length in 108 single rAAV KI models and

FIG. 2C is a scatter plot of KI efficiency vs. Insert length/total homology length. R represents Pearson's correlation coefficient between x- and y-axis values shown.

FIG. 2D is a boxplot comparing HDR efficiency of direct insertion using a single gRNA vs. gene replacement KI models using two gRNAs. n.s. represents not significant with a p-value cut-off of >0.05 for significance.

FIG. 3A is a schematic of sequential insertions using two or three co-delivered rAAV donors.

FIG. 3B is a scatter plot of KI efficiency by junction PCRs vs. insert length in 16 models with two- (blue dots) and three-rAAVs (red dots).

FIG. 4A is a schematic of LOCK-seq.

FIG. 4B is a dotplot of read depth (log 2 normalized) per base across the XCB528 mCherry insertion (schematic on top). Dashed vertical lines (blue) mark boundaries of insert and homology arms. Red arrows point to copy number variation.

FIG. 4C is an IGV screenshot of aligned bam file for Project MS2757 illustrating multiple individual raw reads (grey bars) that cover the length of the cassette and extend into flanking genomic regions for both KI and wild-type alleles. The black horizonal bar across read indicates absence of FKBP-V insert, illustrating the heterozygous state of the F1 animal sequenced. Raw, uncorrected reads are shown, vertical purple bars within reads indicate sequencing error.

FIG. 4D is a dotplot of read depth (log 2 normalized) per base across the longer insert mediated by XCC113 3-rAAV donors. Dashed vertical lines (blue) mark boundaries of genetic regions: gen5, 5′ genomic; L and R, left and right homology arms, CAG, CAG promoter; LSL: floxed polyA signals; Per1, cDNA variants (hashed boxes); pA, polyA signal. Red arrows mark copy number variation.

FIG. 5A is a schematic of an unintended editing outcome in mice: random integration at unrelated, non-target loci.

FIG. 5B is a schematic of an unintended editing outcome in mice: deletion of repetitive element.

FIG. 5C is a schematic of an unintended editing outcome in mice: donor concatenation at the target site.

FIG. 5D is a schematic of an unintended editing outcome in mice; and imprecise KI resulting in additional sequence at 5′ end of transgene.

FIG. 6A is a schematic of probe generation and tiled probes across transgene.

FIG. 6B is a line graph of uniformity of coverage across samples FIG. 6C is a violin plot of lengths (bp) of mapped reads,

FIG. 6D is a graph of total mapped read counts.

FIG. 6E is a graph of capture efficiency (mapped reads/total reads).

FIG. 7A is a bar graph of HDR and NHEJ for BV2 and N2a cell lines using an ssODN template with CRISPR/Cas9. Projects MS2030 and XC10218, are an insertion of a loxP site with a BamHI site; MS2071 and XC101918, are a two base pair change from AG to CC and CC to TG, respectively.

FIG. 7B is a table of KI efficiency of three related 3-rAAV projects, Project XCC113, in BV2 cells. The size of each donor insert (bp) and total number of clones screened, resulting hits, and percent efficiency (hits/total clones) is reported.

FIG. 8 is a workflow schematic of the sequencing methods of the present disclosure. First, reads are aligned to the donor construct (including homology arms), then mapped reads (fastq) are pulled to assemble (fasta) the on-target editing events using Canu. Not all editing events are precise or on-target. When indels or mutations are present, they are evident in the assembly. These donor-specific mapped reads (fastq) will contain overhangs that map to the flanking genomic regions if an on-target event occurred or will have genomic regions that map elsewhere if a random integration occurred. In the second step, the mapped reads (fastq) are mapped to mm39 to determine where in the genome donor-specific reads map. If both on-target and random integrations occurred, high read depth at the on-target site (>40% of total mapped reads) will be identified, in addition to high read depth at the candidate off-target sites (>20% of total mapped reads).

FIG. 9 is a schematic of gene replacement strategy used in the study where two gRNAs are used to excise the region marked for replacement, i.e. concomitant deletion and knock-in (e.g. MS2742/MS2746). The homology arms are designed to flank the replacement region, outside of both cut sites and with 500-1000 bp in length each.

FIG. 10 is a schematic of conditional expression configuration in the ROSA26 locus, inserted with the first rAAV donor of the cassette containing the CAG promoter, floxed 3xSV40 poly A and a BGH poly A signal. The cDNA to be expressed will be inserted into the XCB1202b.sp2 gRNA site on a second donor. Sperm from males harboring this “recipient” site was frozen down to be used in IVT for fertilized eggs so that only rAAV donors delivering cDNAs are needed for future targeting.

FIG. 11A is a schematic of rAAV donor and junction PCR designs of a “failed” project.

FIG. 11B is a gel image of 22 animals positive for internal PCRs specific to cDNA transgenes, and

FIG. 11C is a gel image of 13/22 of internal PCR positive animals also positive for 3′ junction PCR.

FIG. 11D is a table of 5′ junction PCRs attempted and negative results across 22 animals positive for internal and/or 3′ junction.

FIG. 11E is a summary table of PCR results illustrating a labor intensive and inconclusive screening odyssey.

FIG. 12A is a schematic of x3 rAAV donor KI for XCC113-Wt (7.14% at 3/42 F0 pups positive) showing left and right homology arms on either side of the three donor inserts (green, yellow, purple), upstream and downstream (grey) flanking genomic regions. Primers (arrows) for two internal (B-C) and four junction PCRs (A, D, E, F) are shown.

FIG. 12B is a gel image of PCR products for FOs screened by junction A PCR. Arrows point to expected band sizes. Animal 20 was negative for junction A (*) but was maintained as putative hit since positive control also failed to amplify.

FIG. 12C is a gel image of PCR products for FOs screened by junction B PCR. Arrows point to expected band sizes.

FIG. 12D is a gel image of PCR products for FOs screened by junction C PCR. Arrows point to expected band sizes. PCR-based screening can result in incorrectly sized bands (**, junction C high background), double banding (junction C hits), weak bands (\hat{ }), and inconclusive results across difficult to amplify regions. Additionally, it is hard to determine whether all these products were amplified from the same allele.

FIG. 12E is a gel image of PCR products for FOs screened by junction D PCR. Arrows point to expected band sizes.

FIG. 12F is a gel image of PCR products for FOs screened by junction E PCR. Arrows point to expected band sizes. Junction E failed to produce positive band for positive control, suggesting further optimization is required. This junction was not used to identify putative hits.

FIG. 12G is a gel image of PCR products for FOs screened by junction F PCR. Arrows point to expected band sizes.

FIG. 13A is a schematic of x1 rAAV donor KI showing left (red) and right (blue) homology arms on either side of donor KI (green), upstream and downstream (grey, 5′ gen and 3′ gen) flanking genomic regions. Primers (arrows) for 5″ and 3′ ITR-specific amplification are shown.

FIG. 13B is a pair of gel images of PCR products for junction positive FOs screened by each set of primers.

FIG. 13C is a boxplot of percent positive for ITRs of total junction positive animals for 21 projects.

FIG. 14A is a violin plot of mapped read lengths from Cas9-seq compared to LOCK-seq.

FIG. 14B is a table of flow cell (R10.4.1) output for Cas9-seq and LOCK-seq, capture efficiency (mapped reads/total), average read length and number of samples multiplexed on a single flow cell.

FIG. 14C is a screenshot of IGV browser loaded with donor reference sequence and Cas9-seq vs. LOCK-seq bam files zoomed to expected −432 bp expected deletion. Samples are Per1 XCC113 also shown in FIG. 5D, LOCK-seq data shown in FIG. 5D.

FIG. 15A is a cartoon of typical probe design spanning the full-length donor including homology arms (e.g. Allele 2). The homology arms are present on all copies since they are designed to target an endogenous genomic locus.

FIG. 15B is an IGV screenshot of mapped reads show the non-targeted allele (or wild-type) is missing the 2.4 kb insert but has homology arms present while the knock-in allele has the 2.4 kb insert (dashed grey box for Allele 2).

FIG. 15C is a Snapgene screenshot for alignments of contigs for both alleles of a heterozygous KI animal resulting from the breeding of a mosaic FOs to wild-type.

FIG. 16(A-D) is set of Snapgene alignments of contiguous reads spanning the 5′ genomic region upstream of HA-L to 3′ genomic region downstream of HA-R for M24 (FIG. 16A), F19 (FIG. 16B), F20 (FIG. 16C), and F21 (FIG. 16D) that map entire KI cassette (homology arms in yellow, mCherry in red) and flanking genomic sequence.

FIG. 16E is a barplot of full-length contiguous reads mapping to the top (blue) or bottom (red) strands of the donor (and homology arms) and flanking genomic DNA.

FIG. 17 is an IGV screenshot of raw reads aligned to Project MS2757 (see SEQ ID NO: 17-318) donor sequence. Purple arrows along top horizonal region below coverage plot indicate “insertion” events, or more likely sequence error. Each grey bar is a single read with purple vertical lines indicating insertion or error within the read. Deletions are shown as horizonal black bars with numbers indicating the total number of missing bases. Non-sequencing error is supported by multiple reads, as shown for the FKBP-V region, where the F1 sequence is heterozygous with roughly half of the reads containing the FKBP-V sequence and the other half not. Pink outline at the end of the reads indicate soft-clipping of the read where additional sequence beyond what is show fails to align to the reference provided. In most cases, there is adapter sequence beyond that fails to align. The last three reads are described in more detail in FIG. 18 below.

FIG. 18 is an IGV screenshot of raw reads aligned to Project MS2757 (see SEQ ID NO: 17-318) donor sequence. Pile-up of three reads at the bottom with shared soft-clipped regions is due to the partial homology (88.5%) to chromosome 14 (mm10, 88.5% chr14:76636541-76636861, 321 bases). These reads have primary alignments to chr14 with the soft-clipped full-length reads mapping to the same region for all three.

FIG. 19A is a gel image and sequencing data for random integration event upstream of Slc6a15.

FIG. 19B is a gel image and sequencing data for random integration event within the first intron of Cd63.

FIG. 20A is a schematic of concatenation event and primer binding sites (red arrows) for PCR validation.

FIG. 20B is a gel image for validation PCRs showing positive 1.4 kb product for two F2 samples with WT Per1 cassettes.

FIG. 20C is a screen shot of alignments of 5′ and 3′ sequencing results for WT F2.1 (top) and WT F2.2 (bottom).

FIG. 21A is a schematic of two unrelated projects with unique insert (A vs. B) knockin. Each sample carried through each step of LOCK-seq, including barcoding, hybridization, ONT library prep, and ONT sequencing. MinION runs were analyzed to look for fusion reads where one read aligned to both unique insertion events from the two unrelated projects.

FIG. 21B is a graph wherein two independent runs were analyzed, each with different projects and samples. In both cases, the total number of fusion reads across samples was <2%.

FIG. 22A a set of flow cytometry of iPSCs edited with RNPs and a cDNA-GFP donor in rAAV vs plasmid format.

FIG. 22B is a pair of gel images of 5′ and 3′ junctions for 59 single-cell derived iPSC clones from a pool edited with cDNA-GFP donor in rAAV format. Numbers on top of lanes indicate clone IDs positive for 5′ and 3′ junctions, and asterisks indicate those clones only positive for a single junction. Gel image also shown in FIG. 26C to compare to alternative screening method.

FIG. 23A is a pair of gel images of high efficiency and consistence of positive junction PCR amplifications from single-cell BV2 clones transduced with x3 rAAV donors for sequential knock-in of WT Per1. All gels show multiplexed PCR for 3′ junction PCRs (larger 2.6 kb band) between donor 3 and 3′ genomic DNA as well as amplification of a 0.4 kb region of Nsmce3 as a loading control.

FIG. 23B is a pair of gel images of high efficiency and consistence of positive junction PCR amplifications from single-cell BV2 clones transduced with x3 rAAV donors for sequential knock-in of Per1 mutant with a 54 bp deletion. All gels show multiplexed PCR for 3′ junction PCRs (larger 2.6 kb band) between donor 3 and 3′ genomic DNA as well as amplification of a 0.4 kb region of Nsmce3 as a loading control.

FIG. 23C is a pair of gel images of high efficiency and consistence of positive junction PCR amplifications from single-cell BV2 clones transduced with x3 rAAV donors for sequential knock-in of −432 bp deletion. All gels show multiplexed PCR for 3′ junction PCRs (larger 2.6 kb band) between donor 3 and 3′ genomic DNA as well as amplification of a 0.4 kb region of Nsmce3 as a loading control.

FIG. 24A is a schematic showing that both rAAV donors and two RNPs are introduced to the embryos or cells at the same time. The first gRNA cleaves the genomic target site to mediate HDR from the first donor (1^{st} insertion), and successful knockin of the first insert introduces in the second, unique gRNA target site into the allele. The second gRNA target site is not cleavable in the single stranded rAAV genome. The second RNP then cleaves the newly introduced gRNA site, allowing the second insertion to occur via HDR (2^{nd} insertion).

FIG. 24B is a schematic showing that in the absence of the second gRNA, a seamless recombinant rAAV donor could be generated by recombination of ssDNA rAAV donors at regions of shared homology.

FIG. 25A is a schematic of primers used for five junction PCRs listed A-E. Homology arms (yellow bars) of second rAAV donor are annotated.

FIG. 25B is a set of gel images of validation PCRs of candidate hits 1-13, no template control is 14, from HEK293 Ts edited using 2 gRNAs (8 positives out of 76 screened) vs. 1 gRNA (2 positive out of 73 screened).

FIG. 25C is a set of gel images of validation PCRs of candidate hits 1-38, no template control is 39, from iPSCs edited using 2 gRNAs (17 positives out of 211 screened) vs. 1 gRNA (3 positives out of 218 screened). Even though without cleavage by the gRNA brought in by donor 1, the second donor was able to insert at a lower level, implying the possibility of recombination between donors 1 and 2 prior to HDR.

FIG. 25D is a table of types of double rAAV KI in HEK293K and iPSCs.

FIG. 26A is a schematic of rAAV sigle donor used for knock-in in iPSCs with primer binding sites shown used for 5′ and 3′ junction PCRs.

FIG. 26B is a circle plot of mean log 2 coverage across 59 clones screened by LOCK-seq.

FIG. 26C is a gel image of 5′ and 3′ junction PCRs (see also FIG. 11(B-C)).

FIG. 26D is a table of ranking of clones based on highest to lowest mean log 2 coverage showing that LOCK-seq.

FIG. 27A is a workflow schematic for evaluation of eight different probe designs (A-H) in parallel, using samples from two projects: XCE1205a (n=4 FOs) and XCC113 (n=6 F1s).

FIG. 27B is a schematic of various probe designs tested: the same probe length (125 bases) and density (20 bp spacing) against one strand (A), alternating strands (B) and both strands (C), lower density (D-E), longer probe length (F and G), or additional biotin moieties (H).

FIG. 27C is a graph showing probe sets of 125 bases in length designed against the top strand with 20 bp spacing and additional biotin moieties (H) had the highest capture efficiency (total mapped reads/total reads, y-axis), followed by 5′-bitoin modified probes (A), across project XCE1205a.

FIG. 27D is a graph showing probe sets of 125 bases in length designed against the top strand with 20 bp spacing and additional biotin moieties (H) had the highest capture efficiency (total mapped reads/total reads, y-axis), followed by 5′-bitoin modified probes (A), across project XCC113.

FIG. 27E is a graph showing the coverage depth (fraction of total bases on the y-axis versus depth of coverage on x-axis) across XCE1205a was also highest with probe designs A and H.

FIG. 27F is a graph showing the coverage depth (fraction of total bases on the y-axis versus depth of coverage on x-axis) across XCC113 was also highest with probe designs A and H.

FIG. 27G is a graph of coverage versus % GC content binned in 50 bp windows, fraction of bases covered (y-axis) are plotted across increasing % GC bins in XCE1205a. Size of data circle scaled to size of bin (number of loci that fit that % GC). Consistent with reduced overall coverage, lower probe density (D-E) performed worst across GC-rich regions while probes with more biotin moieties achieved the most consistent coverage.

FIG. 27H is a graph of coverage versus % GC content binned in 50 bp windows, fraction of bases covered (y-axis) are plotted across increasing % GC bins in XCC113. Size of data circle scaled to size of bin (number of loci that fit that % GC). Consistent with reduced overall coverage, lower probe density (D-E) performed worst across GC-rich regions while probes with more biotin moieties achieved the most consistent coverage.

FIG. 27I is a graph of read length distribution by test and quartile in XCE1205a. Read length (y-axis, bp) is shown per sample, with split distributions within samples across quartiles.

FIG. 27J is a graph of read length distribution by test and quartile in XCC113. Read length (y-axis, bp) is shown per sample, with split distributions within samples across quartiles.

FIG. 27K is a graph of read length composition in XCC113 by test using global deciles (top 10% and bottom 10% for all samples for each project), plotting the proportion that constitutes the globally calculated deciles for each sample.

FIG. 27L is a graph of read length composition in XCE1205a by test using global deciles (top 10% and bottom 10% for all samples for each project), plotting the proportion that constitutes the globally calculated deciles for each sample.

### DETAILED DESCRIPTION OF THE DISCLOSURE

The protocols disclosed herein address both of the input material and cost bottlenecks described above by using Tn5 to create long barcoded fragments in an unbiased manner that can be amplified and multiplexed for downstream library preparation. Development of a method to multiplex samples for long-read sequencing on the Oxford Nanopore. This method adapts a hyperactive version of Tn5 with three point mutations (E54K, M56A, and L372P) as previously described for fragmentation and barcoding of low-input genomic DNA. The construction of libraries for short-read sequencing on the Illumina platform using Tn5 was published previously. However, alternative reaction conditions had to be established for long-read sequencing of low-input material for the application disclosed herein. In particular, the use of a 5× TAPS-PEG buffer (5×PEG, 0.2 M TAPS, 1 M MgCl2) combined with a 37° C. or room temperature incubation for 15-20 minutes achieves longer fragment sizes (>2-3 kb) of barcoded material, suitable for pre-amplification of low-input material that can be multiplexed for probe-based target capture. The design of the oligos used to pre-load the Tn5 for barcoding have unique barcodes that are flanked by unique universal sequences (e.g., Illumina i5 and 7 sequences) so that a single primer set is used to amplify all samples while maintaining the unique barcode sequence. This process facilitates multiplexing of samples.

### Modulation Agents

As described herein, gene and/or associated protein expression has been implicated in various diseases, disorders, and conditions. As such, modulation of gene and protein expression can be used for treatment of such conditions. A modulation agent can modulate response, such as by inducing or inhibiting gene and/or protein expression signaling. Modulation can comprise modulating protein expression on cells, modulating the quantity of gene/protein expressing cells, or modulating the quality of gene/protein expressing cells.

Modulation agents can be any composition or method that modulates expression on cells. For example, a modulation agent can be an activator, an inhibitor, an agonist, or an antagonist. As another example, the modulation can be the result of gene editing.

A modulation agent can be an antibody (e.g., a monoclonal antibody). A modulating agent can be an agent that induces or inhibits progenitor cell differentiation into gene/protein expressing cells.

Signal Reduction, Elimination, or Inhibition by Small Molecule Inhibitors, shRNA, siRNA, or ASOS

As described herein, a modulation agent can be used for use in various therapies, such as to reduce/eliminate or enhance/increase expression signals. For example, a modulation agent can be a small molecule inhibitor, a short hairpin RNA (shRNA), or a short interfering RNA (siRNA). As another example, RNA (e.g., long noncoding RNA (lncRNA)) can be targeted with antisense oligonucleotides (ASOs) as a therapeutic. Processes for making ASOs targeted to RNAs are well known; see e.g., Zhou et al. 2016 Methods Mol Biol. 1402:199-213. Except as otherwise noted herein, therefore, the process of the present disclosure can be carried out in accordance with such processes.

### Inhibiting Agent

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. The biological component could be an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Chemical Agent

Examples of various sample preparation and assay agents are described herein. Some embodiments include a pharmaceutically acceptable salt, solvate, polymorph, tautomer, prodrug, analog, and/or stereoisomer thereof.

The formulas, analogs, and R groups can be optionally substituted or functionalized with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxyl; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O— cyclopropyl, —O-cyclobutyl, —O-cyclopentyl, —O-cyclohexyl, —O-cycloheptyl, —O-cyclooctyl, —O— cyclononyl and —O-cyclodecyl, —O—CH₂-cyclopropyl, —O—CH₂-cyclobutyl, —O—CH₂-cyclopentyl, —O-CH₂-cyclohexyl, —O—CH₂-cycloheptyl, —O—CH₂-cyclooctyl, —O— CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “heterocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | purine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes. An operon is a cluster of genes that are transcribed together to give a single messenger RNA (mRNA) molecule, which therefore encodes multiple proteins.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence (or polynucleotide sequence) must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using self-replicating primers, paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product-consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by IIe, Leu by IIe, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell geno

| | |
|---|---|
| Side Chain Characteristic | Amino Acid |

Conservative Substitutions I

| | |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| | Ile, Val, Met, Ala, |
| Leu (L) | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, |
| | Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Genome Editing

As described herein, gene and/or protein expression signals can be modulated (e.g., reduced, eliminated, or enhanced) using genome editing.

As described herein, activity, signals, expression, or function can be modulated (e.g., reduced, eliminated, or enhanced) using genome editing (e.g., upregulate, downregulate, overexpress, underexpress, express (e.g., transgenic expression), knock in, knock out, knockdown).

Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of gene/protein expression/signaling by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications to target cells by the removal or addition of signals (e.g., activate (e.g., CRISPRa), upregulate, overexpress, downregulate).

For example, the methods as described herein can comprise a method for altering a target polynucleotide sequence in a cell comprising contacting the polynucleotide sequence with a clustered regularly interspaced short palindromic repeats-associated (Cas) protein.

### Gene Therapy and Genome Editing

Gene therapies can include inserting a functional gene with a viral vector. Gene therapies are rapidly advancing.

There has recently been an improved landscape for gene therapies. For example, in the first quarter of 2019, there were 372 ongoing gene therapy clinical trials (Alliance for Regenerative Medicine, 5/9/19).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies.

| | Strategy |
|---|---|
| Viral Vectors | |
| Retroviruses | Retroviruses are RNA viruses transcribing |
| | their single-stranded |
| | genome into a double-stranded DNA copy, |
| | which can integrate into host chromosome |
| Adenoviruses (Ad) | Ad can transfect a variety of quiescent and |
| | proliferating |
| | cell types from various species and can |
| | mediate |
| | robust gene expression |
| Adeno-associated | Recombinant AAV vectors contain no viral |
| Viruses (AAV) | DNA and can carry ~4.7 kb of foreign |
| | transgenic material. They |
| | are replication defective and can replicate |
| | only while |
| | coinfecting with a helper virus |
| Non-viral vectors | |
| plasmid DNA | pDNA has many desired characteristics as a |
| (pDNA) | gene |
| | therapy vector; there are no limits on the size |
| | or genetic |
| | constitution of DNA, it is relatively |
| | inexpensive to supply, |
| | and unlike viruses, antibodies are not |
| | generated |
| | against DNA in normal individuals |
| RNAi | RNAi is a powerful tool for gene specific |
| | silencing that |
| | could be useful as an enzyme reduction |
| | therapy or |
| | means to promote read-through of a |
| | premature stop |
| | codon |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc., may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to reagents disclosed herein. Such packaging of the components separately can, if desired, be presented in a pack or dispenser device which may contain one or more unit dosage forms containing the composition. The pack may, for example, comprise metal or plasticfoil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit, instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

Multi-kilobase knock-ins (KIs) are a necessary, yet challenging type of genome editing to create and characterize in cell lines and animals. The combination of rAAV donor transduction and electroporation of single-cell mouse embryos with Cas9/gRNA ribonucleoprotein complex (RNP) enables highly efficient KI, but the insert size is limited by the viral packaging capacity. In Example 1, over 100 KI mice created with single rAAV donors are reported on and variables affecting KI efficiency are identified. To overcome the size limit of the rAAV genome, it is shown for the first time that co-delivery of up to three rAAV donors with CRISPR RNPs achieve 6.6 kb precise KI in mouse embryos. To fully characterize the edited genome, LOCK-seq (LOng-read sequencing of Captured Kilo-base targets) was developed. By leveraging biotinylated probe-based hybridization, LOCK-seq achieves >100-fold on-target read depth than previous Cas9-based enrichment methods. The approach simultaneously verifies the precision of KI, detects imperfections in KI, such as partial deletions and insertions as well as donor concatenation, variable sized indels in the non-KI allele and more importantly, localizes random integration of the full or partial donor, which other commonly used methods cannot. Additionally, the multi-rAAV donor approach was applied in cancer and stem cell lines, including BV2 cells where KI was previously thought to be impossible due to intolerance to plasmid DNA. In summary, the targetable insert length is expanded in both mice and cell lines using CRISPR and rAAV donors and a much-needed, sensitive method for surveying both desired targeted integration and undesired, potentially phenotypic-skewing modification events in cell and mouse models is reported.

### Materials and Methods

gRNA Design

gRNAs were designed using an in-house algorithm that incorporates off-target MIT specificity scores and Cutting Frequency Determination (CFD) scores weighted on the homology of the spacer sequence to other sites in the genome. The algorithm also reports SNPs at on-target sites from the Mouse Genomes Project. The selection of gRNAs prioritized higher specificity of spacer sequences with low off-target scores (>70 MIT, >0.2 CFD), proximity of the cut site to the insertion site, and absence of SNPs within the target strain background. Synthetic gRNAs were purchased as Alt-R single gRNAs from IDT (Coralville, Iowa). All gRNAs used for the present disclosure are listed in SEQ ID NO: 17-155.

### AAV Vector and Virus Production

Donors were synthesized by VectorBuilder (Chicago, IL), BioBasic (Amherst, NY), or Genscript (Piscataway, NJ) with flanking canonical AAV2 ITR sequences or cloned into a vector carrying canonical ITRs (5′cctgcaggcagctgcgcgctcgctcgctcactgaggccgcccgggcaaagcccgggcgtcgggcgacctttggtcgccc ggcctcagtgagcgagcgagcgcgcagagagggagtggccaactccatcactaggggttcct-3′, SEQ ID NO: 1) or one canonical and one alternative ITR (5′-aggaacccctagtgatggagttggccactccctctctgcgcgctcgctcgctcactgaggccgggcgaccaaaggtcgcccg acgcccgggcggcctcagtgagcgagcgagcgcgcagctgcctgcagg-3′, SEQ ID NO: 2). All donors used for the present disclosure are listed in SEQ ID NO: 156-318. Production of rAAV6 virus was performed by the Hope Viral Vectors Core (Washington University, St. Louis, MO) using iodixanol gradient purification, or from VectorBuilder (Chicago, IL) or Genscript (Piscataway, NJ) as a crude preparation from cell-free medium. A difference in efficiency was not observed between the two types of preparations.

Validation of sgRNAs and rAAV Donors in N2a Cells

N2a cells were cultured in DMEM media +10% FBS, +1% Glutamax, +1% Pen-Strep at 37° C. in a humidified incubator with 5% CO₂ and passaged using 0.25% Trypsin-EDTA using standard cell culture techniques. Cas9/gRNA RNPs were complexed at room temperature by mixing 1 μl of recombinant Cas9 protein (40 μM, from QB3 MacroLab, UC Berkeley) with 1 μl of each sgRNA (100 μM). Nucleofections were performed using 150,000 cells in 20 μl OPTI-MEM or P3 solution and DS-137 program. After nucleofection, cells were immediately seeded in a well of a 24-well plate with 500 ul of growth medium supplemented with 1 μl of each rAAV donor and harvested 72 hr later for genotyping. All reagents for mouse projects were validated in N2a cells first before being used in mouse production.

### Amplicon NGS for CRISPR Cleavage Activity at the Target Site

Transfected N2a cells or tail clips of mice were lysed in QuickExtract Solution (Biosearch Technologies, cat. #QE09050), following manufacturer's instructions. The target regions were PCR amplified by tailed primers appended with 5′-CACTCTTTCCCTACACGACGCTCTTCCGATCT-3′ (SEQ ID NO: 3) for forward and 5′-GTGACTGGAGTTCAGACGTGTGCTCTTCCGATCT-3′ (SEQ ID NO: 4) for reverse to genomic-specific primer sequences (PCR 1), which allows unique indexes and Illumina P5/P7 adapter sequences to be added in a second round of PCR. All primer sequences are listed in SEQ ID NO: 319-376. PCR amplifications were performed with JumpStart REDTaq ReadyMix (MilliporeSigma, cat. #P0982), following the manufacturer protocol. The following cycling conditions were used: 94° C. for 2 min, followed by five cycles of 94° C. for 30 s, 54° C. for 30 s, and 72° C. for 40 s. 2×250 reads were generated with the Illumina MiSeq platform at the Center for Genome Sciences and Systems Biology (Washington University, St. Louis, MO). The extracted FASTQ files are analyzed using a Python-based script which outputs editing efficiency.

### Junction PCRs to Confirm Targeted Integration

The same N2a lysates were used as templates for junction PCRs for detecting knock-in events. All junction PCR primers span flanking genomic and cassette-specific sequence or junctions between rAAV-specific cassettes when using multiple rAAV donors. PCR amplification was performed using primers listed in SEQ ID NO: 319-376 with Platinum SuperFi II Green PCR Master Mix (ThermoFisher, cat. #12369050) or JumpStart REDTaq ReadyMix (MilliporeSigma, cat. #P0982).

### Mouse Husbandry and Embryo Manipulation

All animals at Washington University in St. Louis are housed under SPF barrier conditions in AALAC-accredited facilities. All required breeding, experiments and interventions are included in IACUC approved protocols. The Department of Comparative Medicine provides basic husbandry in accordance with their procedures. Three to four weeks-old C57BL/6J mice (JAX Laboratories, Bar Harbor ME, USA) were superovulated by intraperitoneal injection of 5 IU pregnant mare serum gonadotropin, followed 47 h later by intraperitoneal injection of 5 IU human chorionic gonadotropin (PMS from SIGMA, HGC from Millipore USA). Mouse zygotes were obtained by mating C₅₇BL/6J stud males with superovulated C₅₇BL/6J females at a 1:1 ratio. rAAV transduction of zygotes was performed as previously reported. Briefly, 10⁹-10¹⁰ viral particles were added to each KSOM droplet containing 20-30 zygotes covered with paraffin oil and allowed to incubate for 5-6 hours in a humidified tissue culture incubator (37° C., 5% CO₂). The zygotes were then washed and electroporated with Cas9/sgRNA complex as previously described. Briefly, one-cell fertilized embryos were electroporated with RNPs containing 12 μg of Cas9 protein complexed with 1:1.5 molar ratio of gRNA. Between 30 to 40 post-transduction zygotes in 10 μl of OPTI-MEM are mixed with 10 μl of RNP in OPTI-MEM before being loaded into a 1 mm electroporation cuvette. With a Biorad Gene Pulser Xcell electroporator, 2-6 pulses of 30V were used for 3 ms with 100 ms internals. Post-electroporation embryos were kept in 25 μl drops of KSOM overlayed with oil and in a humidified tissue culture incubator (37° C., 5% CO₂) for 1-2 hours before being surgically transferred to 0.5d PC pseudopregnant surrogates.

For multiple rAAVs, all rAAV donors were combined at equal number of viral particles before adding onto the zygotes. After incubation, the washed zygotes were electroporated with all necessary RNPs at equal molar ratio, with the total RNP mass per electroporation held constant at 12 ug.

Founder genotyping by junction PCRs and detection of random integration by ITR PCRs Founder genotyping was done using conditions optimized during validation. A primer used for the detection ITR-containing donor random integration binds to the hairpin of the ITR, 5′-TGGCCAACTCCATCACTAGG-3′ (SEQ ID NO: 5 and SEQ ID NO: 377-816, XCC216c.AAV2.ITR.F2-R2) and pairs with an insert specific primer at each end of the donor.

Cas9-Targeted Sequencing (nCATS)

Genomic DNA from tail clips was isolated with the Monarch Genomic DNA Purification Kit (NEB, cat. #T3010S). nCATS was performed using the Oxford Nanopore's Cas9 Sequencing Kit (cat. #SQK-CS9109) according to the manufacturer's instructions. Briefly, 5 μg of extracted DNA was 5′ dephosphorylated and cut with a pair of RNPs (5′-TAAGGGAGCTGCAGTGGAGT-3′ (SEQ ID NO: 6), 5′ GGATTTAGCCACATCCATAG-3′ (SEQ ID NO: 7)) flanking the region of interest. RNPs were each prepared using 100 μM of gRNA complexed with 30 μM of Cas9 protein (QB3 MacroLab, UC Berkeley) and added in a dA-tailing cocktail at 37° C. for 35 min. Adapter ligation and clean-up was performed using the Ligation Sequencing Kit V14 (Oxford Nanopore Technologies, cat. #SQK-LSK114) and the final library run on an R10.4.1 MinION flow cell (Oxford Nanopore Technologies, cat. #R10.4.1).

### LOCK-Seq (LOng-Read Sequencing of Captured Kilo-Base Targets)

Genomic DNA was isolated from tissue and cell pellets using the Monarch Genomic DNA Purification Kit (NEB, cat. #T3010S) or with QuickExtract DNA Extraction Solution (Biosearch Technologies, cat. #QE0901 L), according to the respective manufacturer's instructions.

### Tagmentation

Tagmentation with Tn5 LongPlex Long Fragment Multiplexing Kit (seqWell, cat. #301312) was used according to the manufacturer's protocol or an in-house protocol for multiplexing. For in-house tagmentation, Tn5 protein (QB3 MacroLab, UC Berkeley) was complexed with annealed oligonucleotide adapters. Briefly, 16.5 uM of adapter primers (Tn5ME-A 5′-TCGTCGGCAGCGTCAGATGTGTATAAGAGACAG-3′ (SEQ ID NO: 8) and Tn5ME-B 5′-GTCTCGTGGGCTCGGAGATGTGTATAAGAGACAG-3′ (SEQ ID NO: 9), each annealed with Tn5MErev 5′-[phos]CTGTCTCTTATACACATCT-3′ (SEQ ID NO: 10) was added to Tn5 protein (500 ng/μl) and incubated at room temperature, shaking at 200 rpm. Tagmentation was performed with 10 μl of crude lysate in 10 mM TAPS-NaOH, pH 8.5, 5 mM MgCl₂, and 7% PEG 8000 in a total reaction volume of 20 ul for 5 min at 55° C. The resulting fragmented genomic DNA was cleaned up using 0.7× JetSeq Clean magnetic beads (Meridian Biosciences, cat. #BIO-68032) and eluted in 20 μl. Barcodes were introduced by PCR using KOD Xtreme Hot Start DNA Polymerase (MilliporeSigma, cat. #71975-3), with primers specific to the universal adapter tail sequences introduced by Tn5. Each PCR reaction used the full volume of fragmented DNA from the previous step with 2 mM of each dNTP and 10 μM of each primer in a final volume of 50 μl. Cycling conditions were 94° C. for 2 min, 8 cycles of 98° C. for 10 sec, 68° C. for 10 min, and a final 72° C. hold for 10 min.

### Mechanical Fragmentation

Up to 5 μg of genomic DNA prepared by using the Monarch kit was loaded onto a Covaris g-TUBE (Covaris, cat. #520079) and centrifuged at 4900×g for 1 min to generate fragments in the 10 kb size range. Fragmented DNA was end-repaired and A-tailed using the TWIST Mechanical Fragmentation Kit (TWIST, cat. #101281), ligated to TruSeq compatible adapters (TWIST, cat. #101310) and barcoded using PCR with KOD Xtreme Hot Start Polymerase, following the manufacturer's protocols. Specifically, the cycling conditions include 94° C. for 2 min, 8 cycles of 98° C. for 10 sec, 58.8° C. for 30 sec and 68° C. for 10 min, and a final 68° C. hold for 10 min. Indexed samples were pooled to a final mass of 4-6 ug for hybridization.

### Biotinylated Probes

Custom capture probes were acquired from xGen IDT or generated in-house using a two-step PCR and tiled at densities of 1-4 probes/kb (commercially purchased probe sequences listed in SEQ ID NO: 377-816).

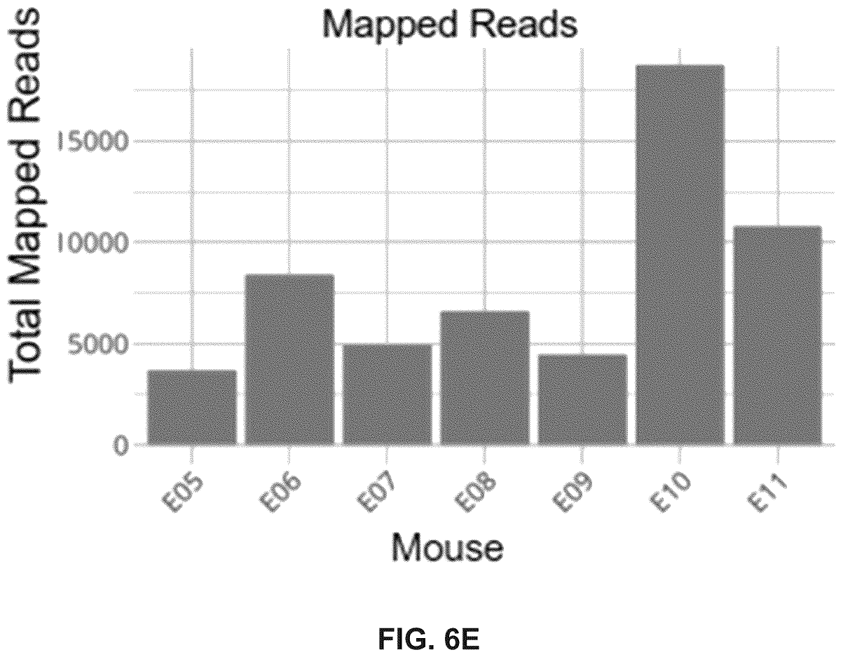

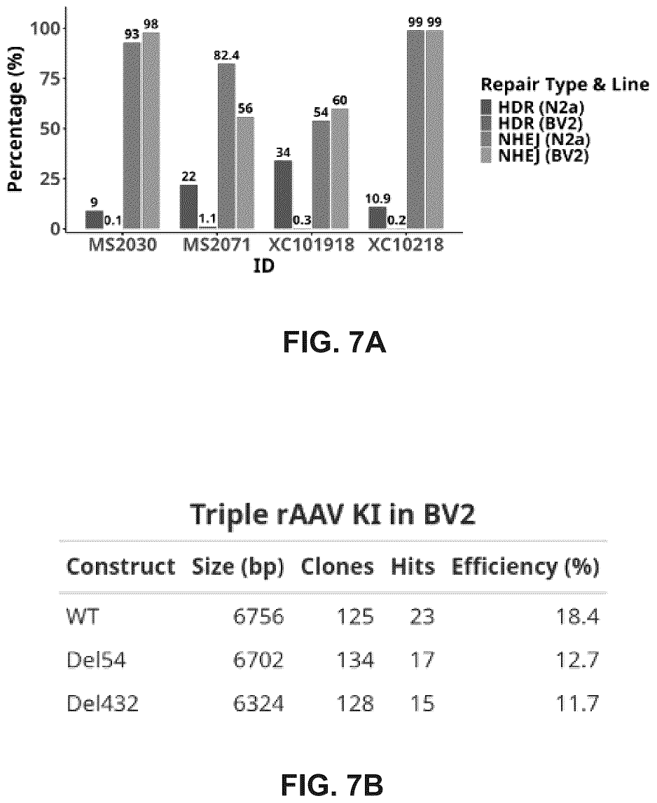

In-house generated LOCK-seq probes presented in FIG. 7(A-B) used tailed primers appended with 5′-CACTCTTTCCCTACACGACGCTCTTCCGATCT-3′ (SEQ ID NO: 11) for forward and 5′-GTGACTGGAGTTCAGACGTGTGCTCTTCCGATCT-3′ (SEQ ID NO: 12) for reverse to genomic-specific primer sequences to target regions, followed by a second step asymmetric PCR with universal primers appended with a 5′ biotin for the forward primer (forward 5′Biotin-CACTCTTTCCCTACACGACGCTCTTCCGATCT 3′ (SEQ ID NO: 13), reverse 5′ GTGACTGGAGTTCAGACGTGTGCTCTTCCGATCT-3′ (SEQ ID NO: 14)). The asymmetric PCR with universal primers were appended with a 5′ biotin for both primers (forward 5′Biotin-agtcaggtgcccaatgtacc-3′ (SEQ ID NO: 15), reverse 5′-Biotin-taggcagttcgctccaagc-3′ (SEQ ID NO: 16)). Primer sequences for all in-house generated probes are listed in SEQ ID NO: 817-1002. PCR amplifications were performed with JumpStart REDTaq ReadyMix (MilliporeSigma, cat. #P0982), according to the manufacturer's protocol. The asymmetric PCR was performed using 0.1× volume of PCR product (no clean-up step) from step1 with the forward primer at 10 μM and reverse primer at 0.5 μM and melting at 94° C. for 2 min, followed by twenty cycles of 94° C. for 30 s, 60° C. for 10 s, and 72° C. for 20 s. Amplicons were pooled and cleaned-up using the Zymo DNA clean and Concentrator kit (Zymo Research, cat #.D4004).

To generate multiple biotin moieties across probes during the asymmetric PCR step, KOD Xtreme Hot Start DNA Polymerase was used with 2 mM each dATP, dCTP, and dGTP, 1.6 mM dTTP and 0.4 mM Biotin-11-dUTP (ThermoFisher cat. #R0081), with the forward primer at 10 μM and reverse primer at 0.5 μM and melting at 98° C. for 2 min, followed by 20 cycles of 98° C. for 10 s, 60° C. for 10 s, and 68° C. for 20 s.

### Hybridization

Multiplexed samples were hybridized with biotinylated probes using Twist Standard Hyb and Wash Kit (cat. #104446), and hybridization reactions for 16-18 hrs were performed according to the manufacturer's protocol for TWIST. Briefly, the pooled DNA was blocked with Universal Blockers (TWIST, cat. #100578) and 5 μg of Mouse Cot-1 DNA (Invitrogen, cat. #18440016) to minimize non-specific capture in the hybridization reagent. Blocking was performed by heating the mixture at 95° C. for 5 minutes, cooling at room temperature for 5 min, and proceeding immediately with the 16-18 hr at 70° C. overnight hybridization with the biotinylated probe panel.

### Post-Hybridization Enrichment

Target-captured libraries were enriched following the TWIST (catalog #104446) post-capture workflow. Briefly, biotinylated DNA-probe hybrids were captured with Dynabeads M-270 Streptavidin (ThermoFisher, cat. #65305) at 68° C. for 10 minutes. Beads were washed per manufacturer's instructions, and bound DNA was released with 0.2 N NaOH. The eluate was neutralized and used directly for post-capture PCR with KOD Xtreme Hot Start DNA Polymerase using TWIST universal adapter primers. Thermocycling conditions were 94° C. for 2 min, 18 cycles of: 98° C. for 10 sec; 58.8° C. for 30 sec; 68° C. for 10 min, with a final extension of 68° C. for 10 min. PCR products were purified by DINOMAG SPRI (Labscoop, cat. #DN9004-5ML) bead cleanup at 0.5× bead:sample ratio.

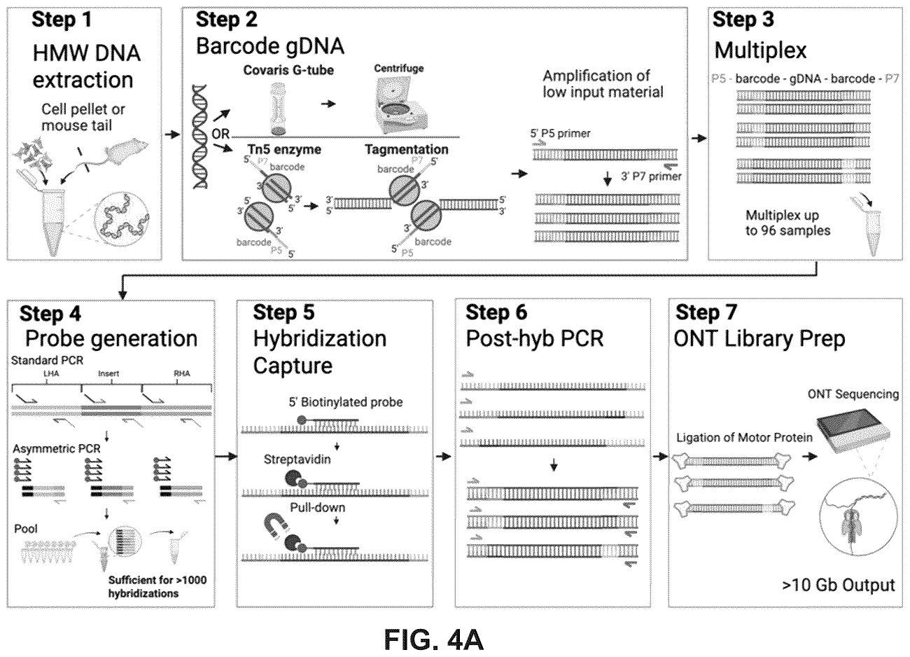

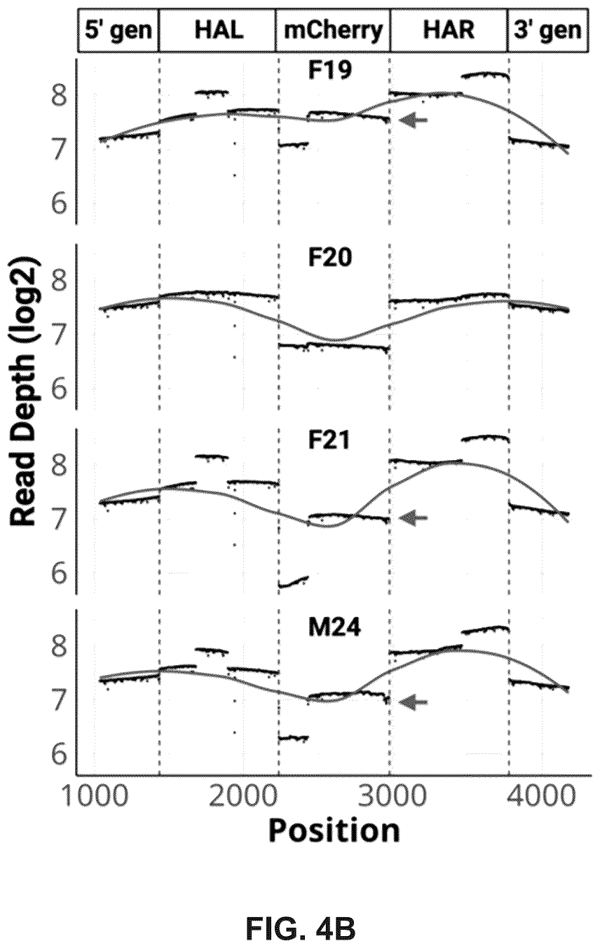

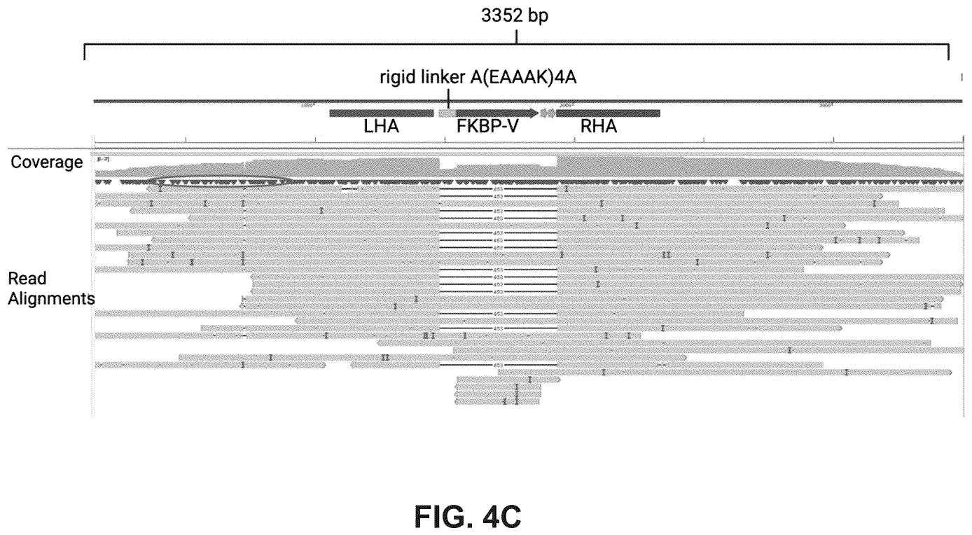

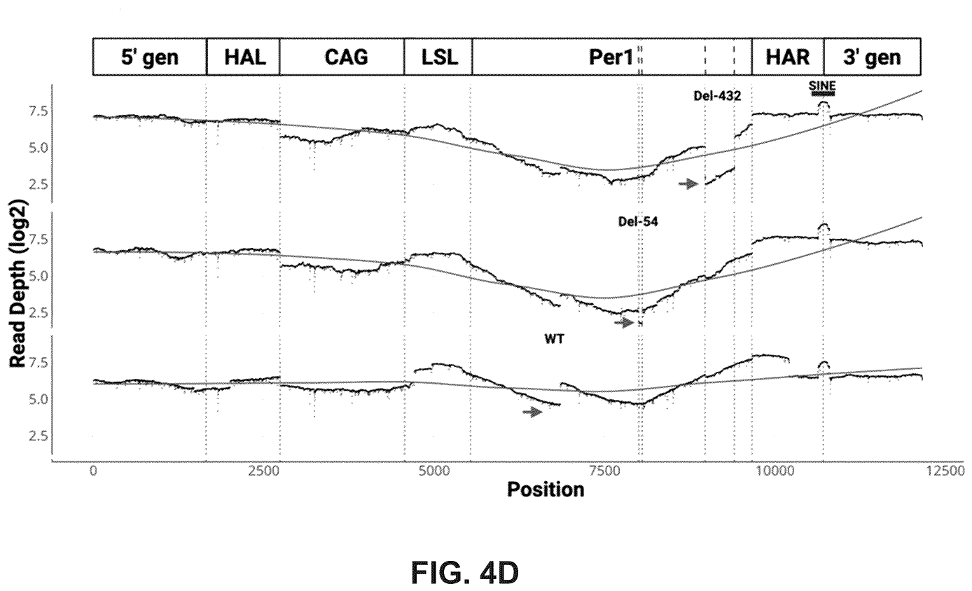

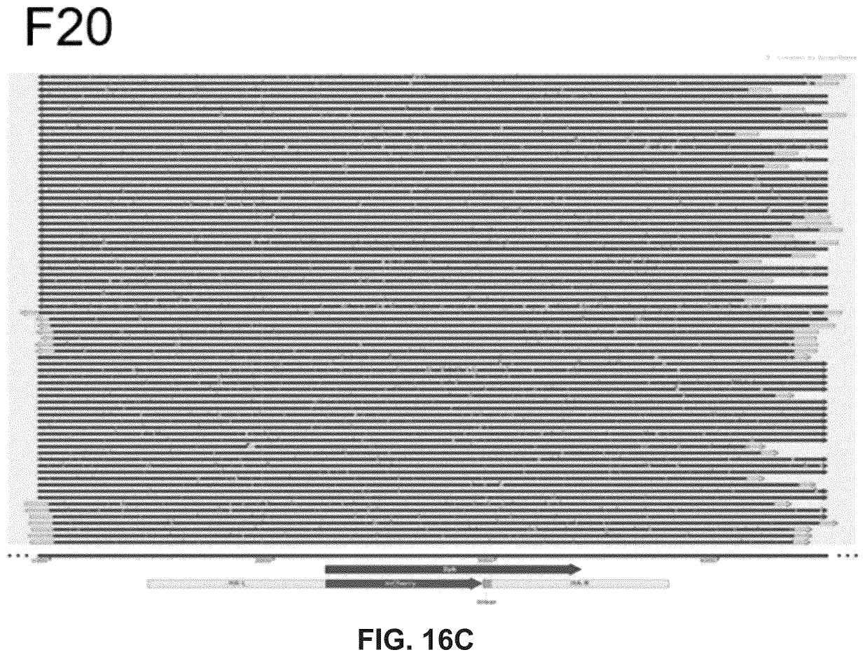

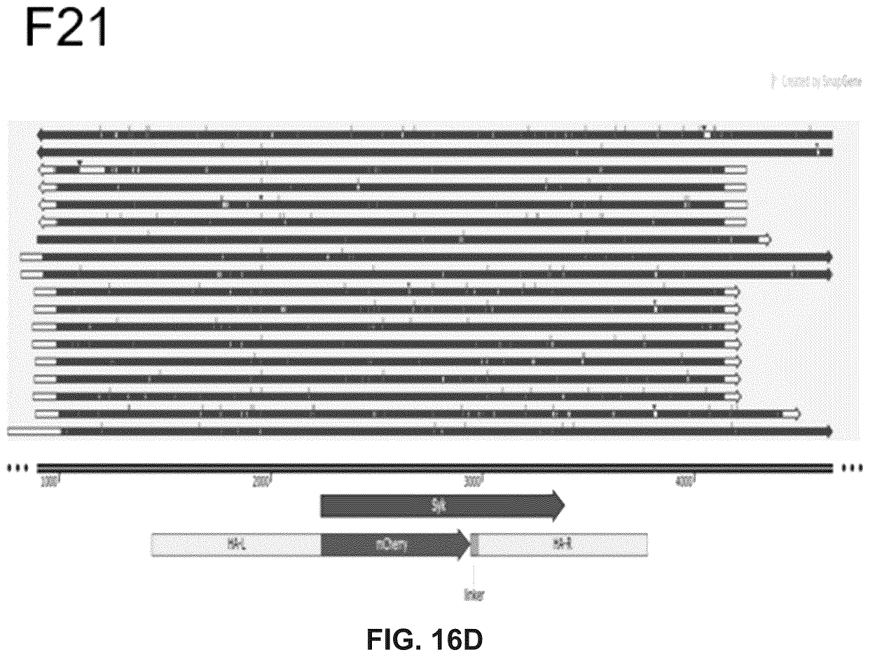

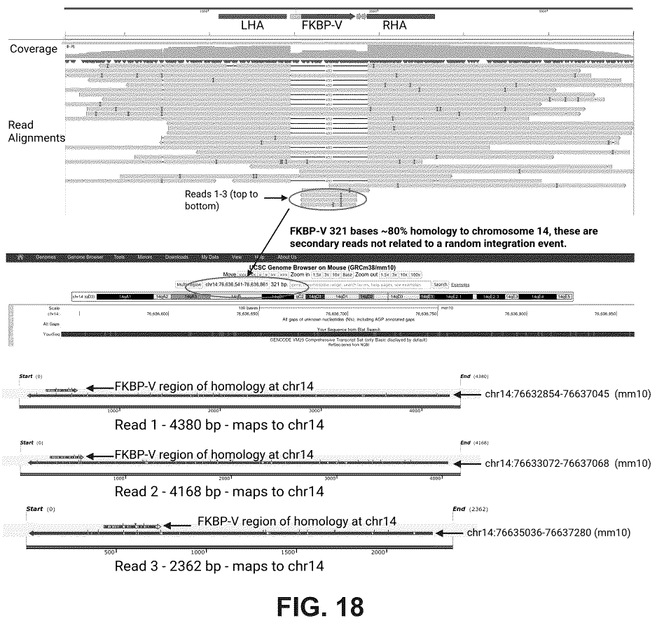

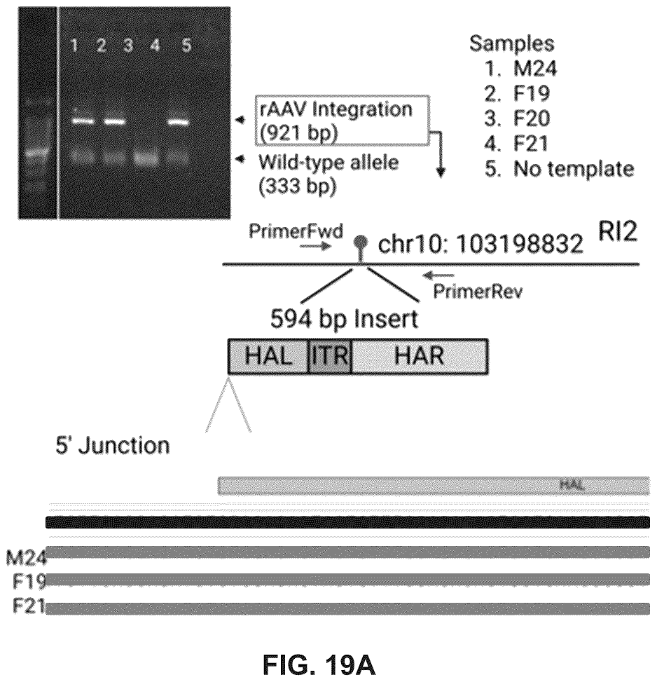

For FIG. 4B, FIG. 16(A-E), and FIG. 19(A-B) (Project XCB528) a double hybridization was performed using the output from the first round as the input for a second round of hybridization, repeating the process with a fresh aliquot of probes. This can be performed to increase the percent of on-target reads.

### ONT Library Preparation

Ligation of Oxford Nanopore sequencing adapters was performed for all libraries using the Ligation Sequencing Kit V14 (Oxford Nanopore Technologies, cat. #SQK_LSK114) and loaded onto an Oxford Nanopore Technologies flow cell (FLO-MIN114 or FLO-PRO114HD).

### LOCK-Seq Analysis

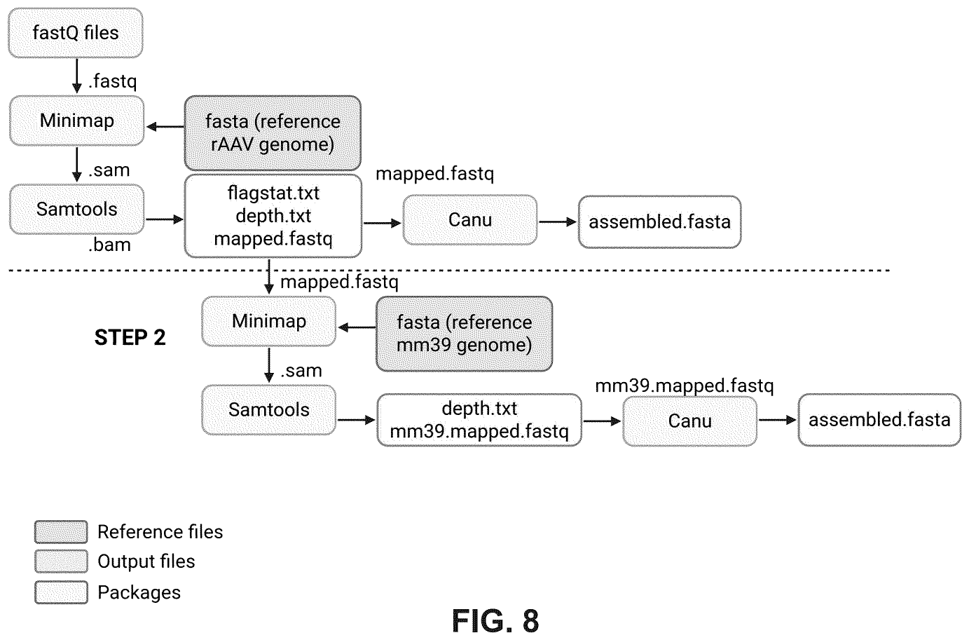

Base calling was performed using Guppy or Dorado (v0.7), and samples were demultiplexed with Nanoplexer or an in-house python script. The code for demultiplexing and the analysis pipeline will be deposited at github.com/msentmanat/LOCK-seq-analysis-pipeline. Briefly, no trimming was performed, and for the in-house demultiplexing script, exact index matches for both indexes were required. Reads with >1 match for either index or lacking one index were excluded. Capture efficiency was determined using flagstat output (total reads mapped to transgene/total reads) and length of mapped reads calculated using FASTQ files derived from transgene mapped BAM files for each sample. FASTQ files were aligned to the transgene first using Minimap2 (v2.28), mapped reads (.fastq) were pulled using Samtools (v1.20), and then mapped to the reference genome (mm39) using Minimap2 (v2.28) and Samtools (v1.20). Using the Samtools depth command, the top 10 genomic coordinates (based on highest read depth, ˜20 kb window) were used to generate a bed file to pull reads from the bam files. Those regions were investigated by manual curation on IGV and Canu to identify random integration events (FIG. 8). FASTQ files were used to build consensus sequences using Canu (v2.2) and visualized using Snapgene. The per base coverage was plotted with normalized reads from Samtools depth output in R.

Gene editing in N2a, BV2, HEK293T, and iPSCs N2a, BV2 and HEK293T cells were cultured in DMEM media +10% FBS, +1% Glutamax, +1% Pen-Strep at 37° C. in a humidified incubator with 5% CO₂ and passaged using 0.25% Trypsin-EDTA using standard culture conditions. iPSC cells were cultured on Matrigel-coated (Corning, cat #0.354277) 6-well plates with mTeSR Plus (STEMCELL Technologies, cat. #100-0276) media at 37° C. in a humidified incubator with 5% CO₂, and passaged using ReLeSR (STEMCELL Technologies, cat. #100-0483) passaging reagent. Cas9 RNPs were complexed at room temperature by mixing 1 μl of recombinant Cas9 protein (40 μM, from QB3 MacroLab, UC Berkeley) with 1 μl of each sgRNA (100 μM). Nucleofections were performed using 150,000 cells per 20 μl reaction (N2a and HEK293T), or one confluent well of a 6-well plate was used for 4-6 reactions (iPSCs). Cells were detached from tissue culture substrate, washed twice with PBS, then resuspended into Opti-MEM media (N2a and HEK293T) or P3 solution (Lonza, cat. #V4SP-3096 for iPSCs) and nucleofected using a Lonza 4D nucleofector with standard programs (N2a: DS-137, HEK293T: CM-130, iPSC: CA-137).

Nucleofected N2a, BV2 and HEK293T cells were seeded into 500 μl of complete culture medium in a well of a 24-well tissue culture plate, supplemented with 1 μL of each AAV donor, and harvested 72 h post-nucleofection for genotyping and expansion for sorting. Nucleofection iPSC cells were seeded into 500 μl of mTeSR Plus media supplemented with 10% CloneR2 (STEMCELL Technologies, cat. #100-0691) for 24 h, and the media was changed to regular mTeSR Plus media for the next 48 h. Nucleofected pools were propagated under standard conditions until genotyping was confirmed. All reagents for mouse projects were validated in N2a cells first before used in mouse production.

To generate clonal edited lines, pools were single-cell dissociated with 0.25% trypsin (N2a, BV2 and HEK293T cells) or 0.75× TrypLE (Fisher Scientific, cat. #12-604-013, iPSC cells), and single cells were sorted into 96-well tissue culture plates on a Sony SH800 cell sorter. N2a cells were sorted into complete media supplemented with 10 μM ROCK inhibitor (MilliporeSigma cat #SCM075) and 1 mM sodium pyruvate (ThermoFisher, cat #11360070) while HEK293T cells were sorted into 50% conditioned media, and iPSCs were sorted into mTeSR Plus supplemented with 10% CloneR2. Clones were monitored for growth, with changes of complete media every 3-5 days (N2A and HEK293T) or 2-3 days (iPSCs) until >40% confluent, then harvested, consolidated, and genotyped by junction PCRs as in described above.

### Results

rAAV Donors Mediate Efficient HDR in Mouse Embryos

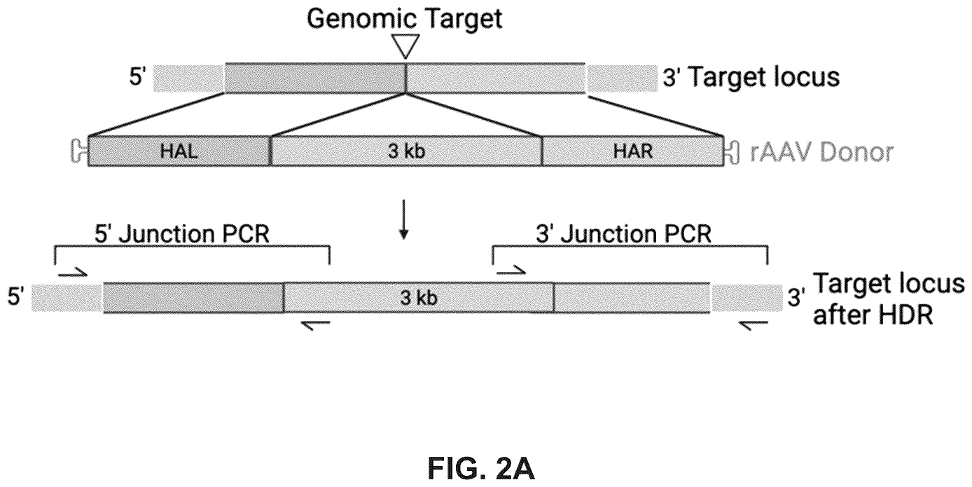

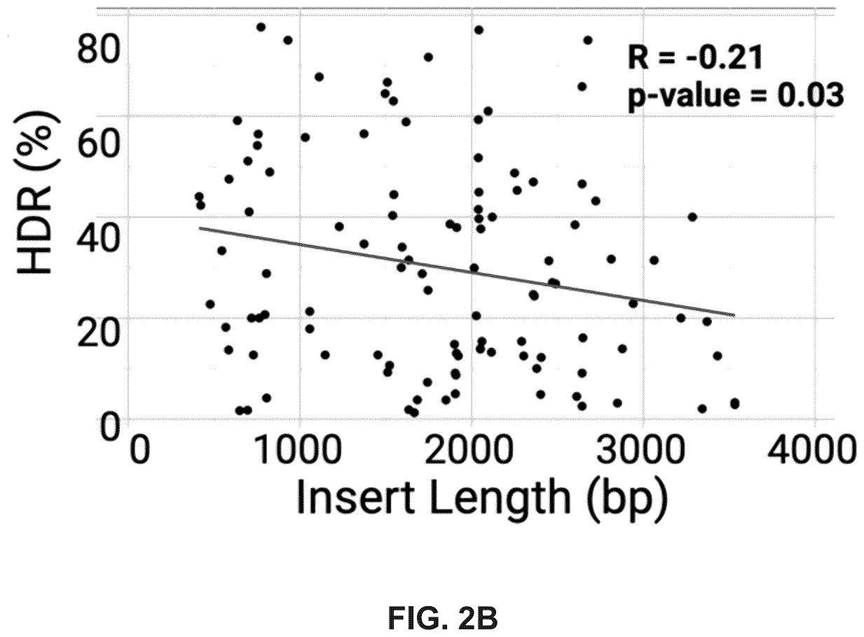

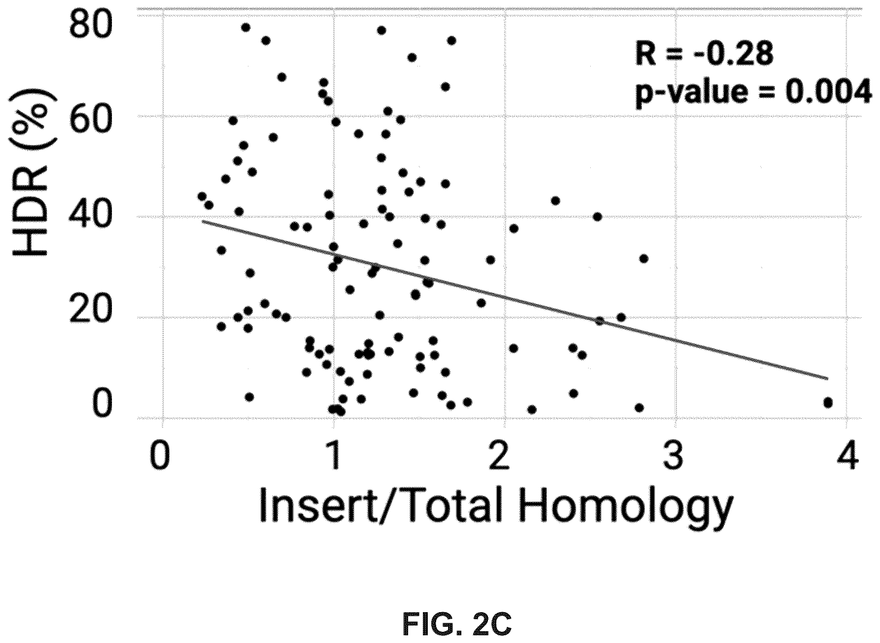

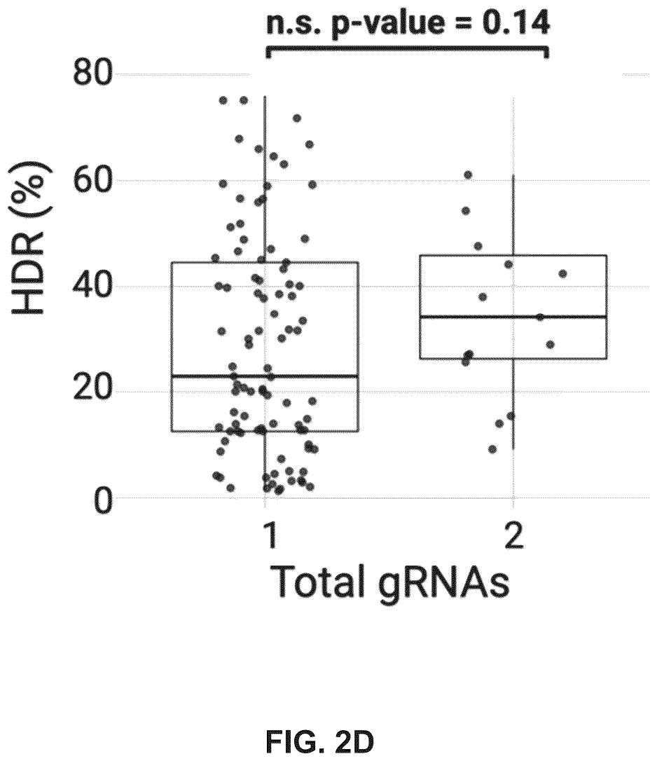

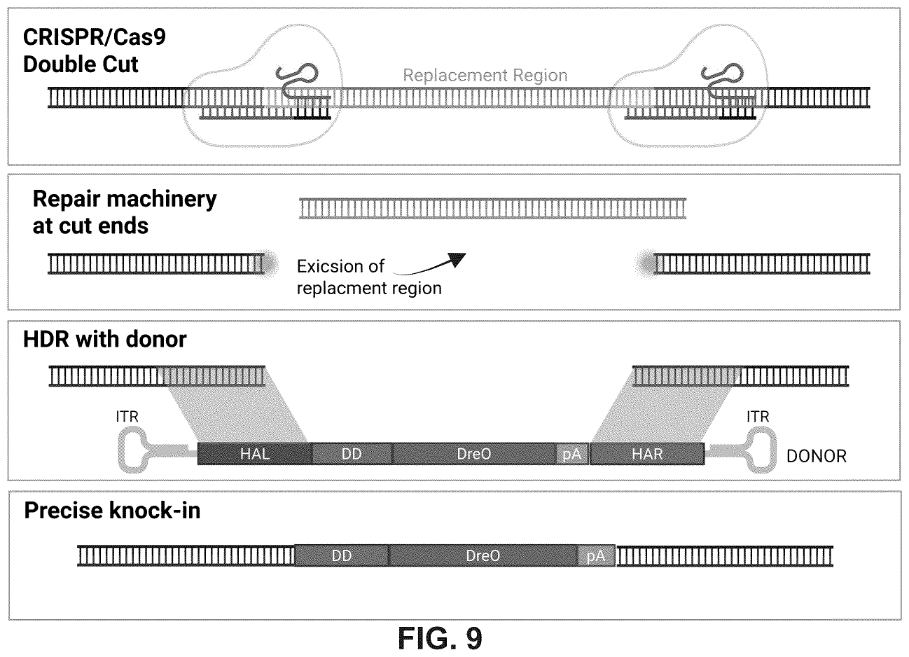

By transduction of mouse embryos with a single rAAV donor, followed by Cas9/gRNA RNP electroporation, 108 mouse models were successfully created with insert sizes ranging from 400 bp to 3.5 kb (FIG. 2A). Founder rates among live births were 1% to 80%, identified by junction PCRs (FIG. 2B). There is a trend that the longer the insert, the less efficient the insertion. However, the strongest correlation is between KI efficiency and length of insert/total homology: each doubling of homology length relative to the insert results in a 30% or greater increase in efficiency of generating founders (R=−0.28, p-value=0.004; FIG. 2C). Fifteen out of the 108 models were gene replacements, generated using one donor and two gRNAs that excise the region targeted for replacement while facilitating HDR (FIG. 9). Gene replacement was as efficient as direct insertion among these models (FIG. 2D).

### Sequential Insertion of Multiple rAA V Donors for Larger Inserts

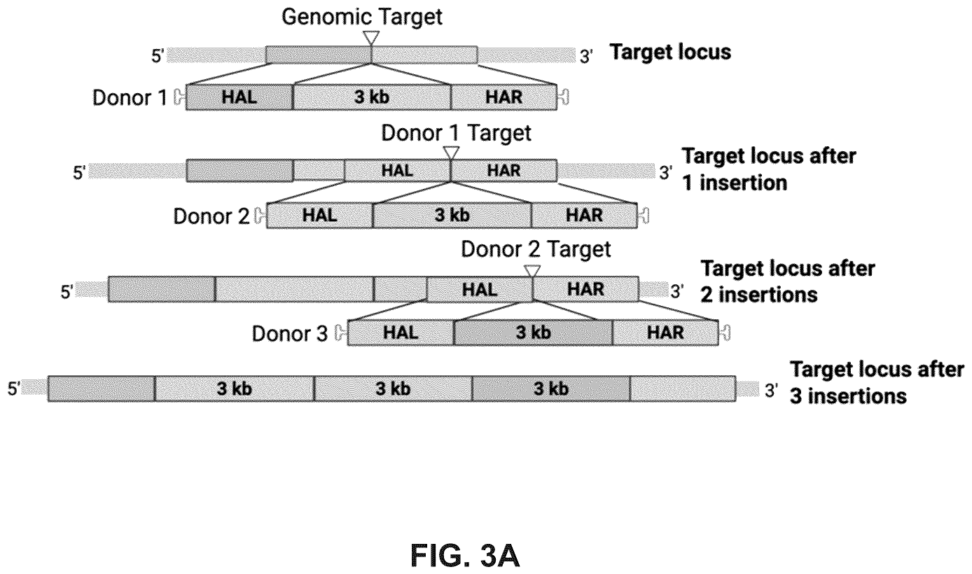

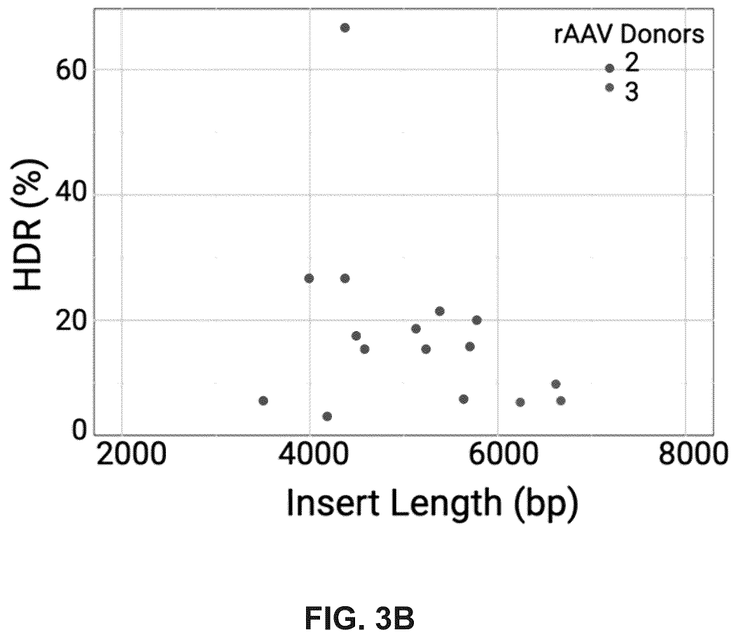

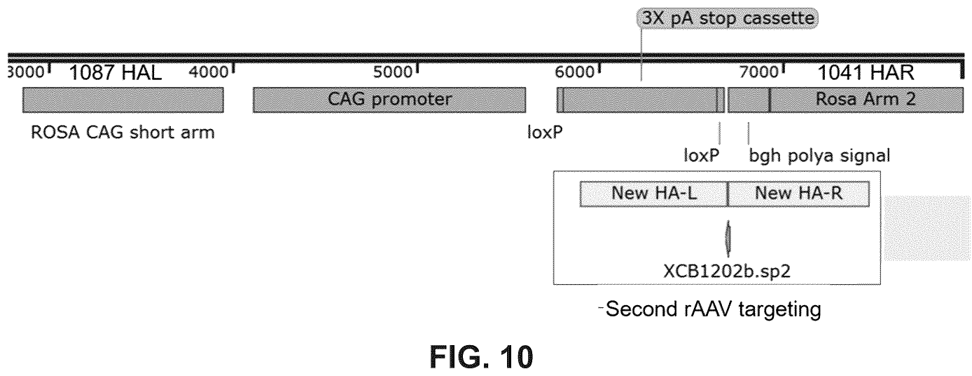

To overcome the 4.7 kb size limitation of an rAAV genome, a sequential insertion strategy was designed, where the first donor delivers a portion of the insert along with a gRNA target site, to mediate cleavage and subsequent insertion from the second donor (FIG. 3A). The gRNA target site in the donor can be identical to the genomic target site, or unrelated, such as a previously validated site from a different species or a new site created by the insertion. The gRNA target is not cleavable in the single-stranded rAAV genome. For even larger inserts, the second rAAV donor brings in an additional gRNA target site to mediate the third insertion (FIG. 3A). All rAAV donors and RNPs are delivered to single-cell embryos in a single step. The consecutive HDR events happen one after the other upon respective CRISPR cleavage within the embryos. To date, 13 models with two-rAAVs and 3 with three-rAAVs have been completed (FIG. 3B). All but one of the multi-rAAV models are targeting the mouse ROSA26 locus. Seven of the two-rAAV-donor and 3 of three-rAAV-donor projects are for conditional expression of a given cDNA from the ROSA26 locus, using the configuration CAG-loxP-3xSV40 pA-loxP-cDNA-pA. Given the cassette CAG-loxP-3xSV40 pA-loxP-pA is common for all, a rAAV donor was created to first insert this common cassette (FIG. 10) and a second (and a third if needed) rAAV will deliver the cDNA. The homology arms were identical for the second donors among different projects. Efficiency ranges from 5 to 67% for 2-rAAV projects were achieved, and about 5% for three-rAAV projects. Of the 133 projects, nine projects failed the first two sessions without obvious reasons, five with single rAAV donors, three with two rAAV donors, and one with three rAAV donors.

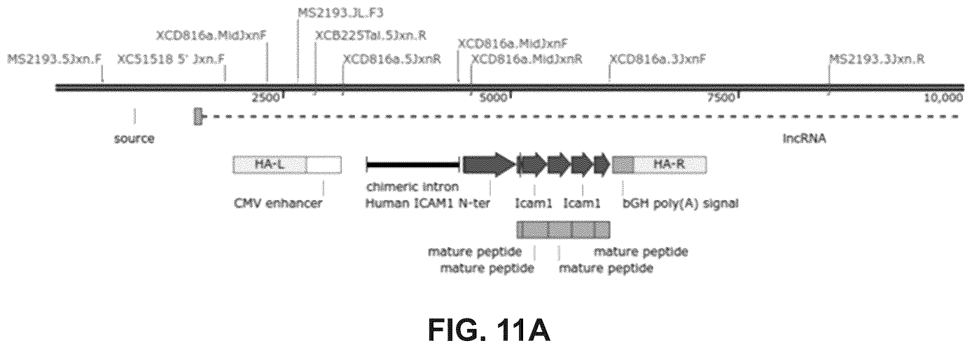

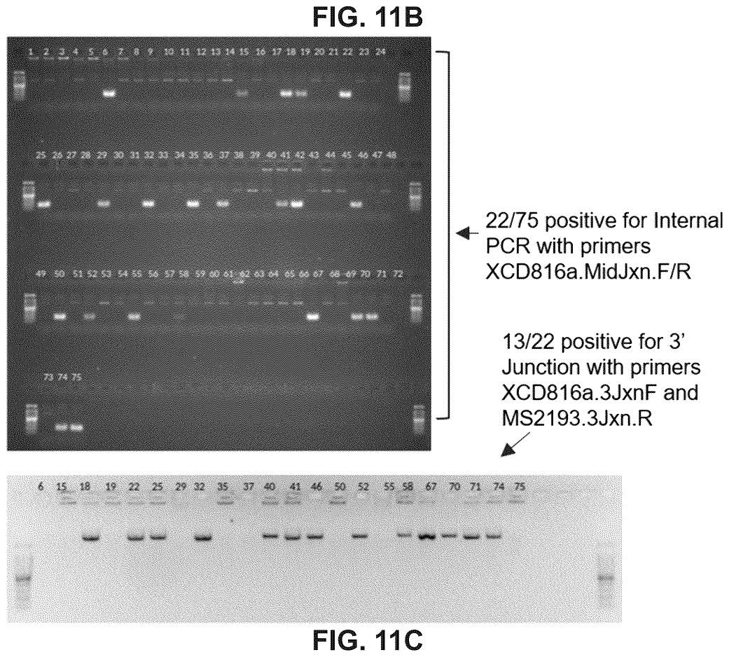

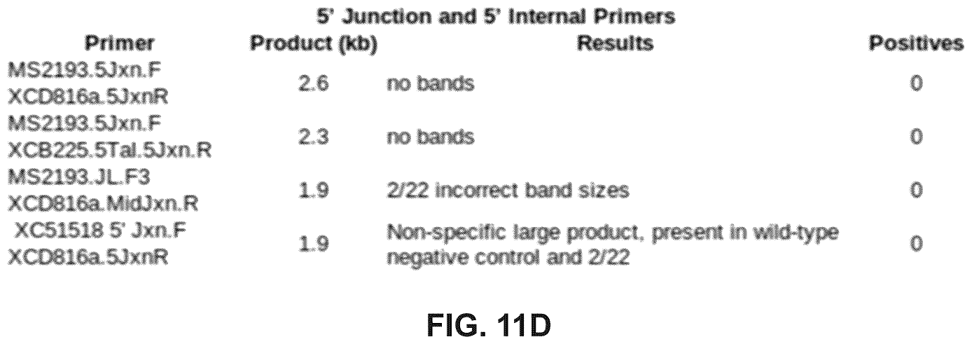

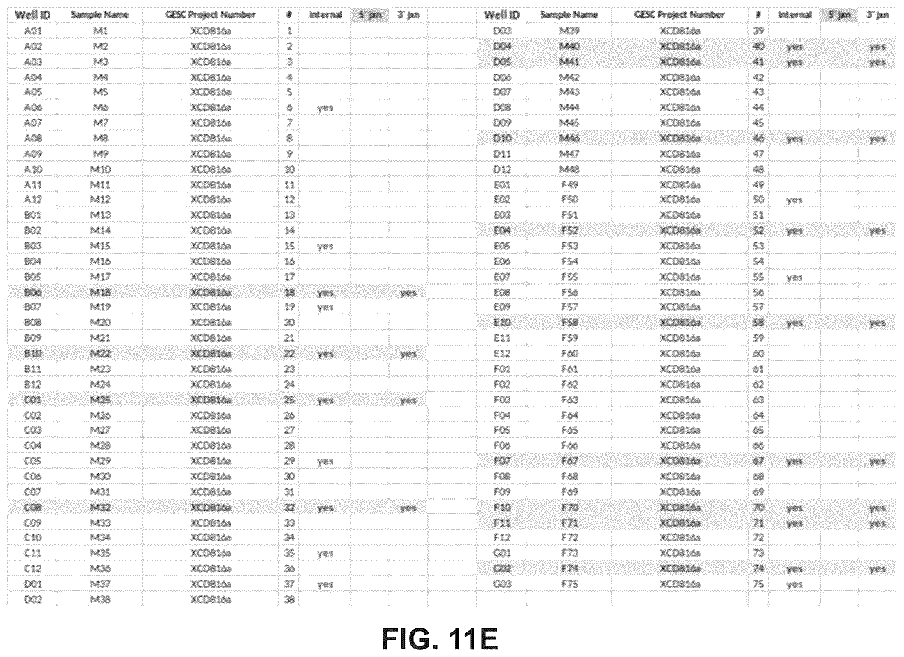

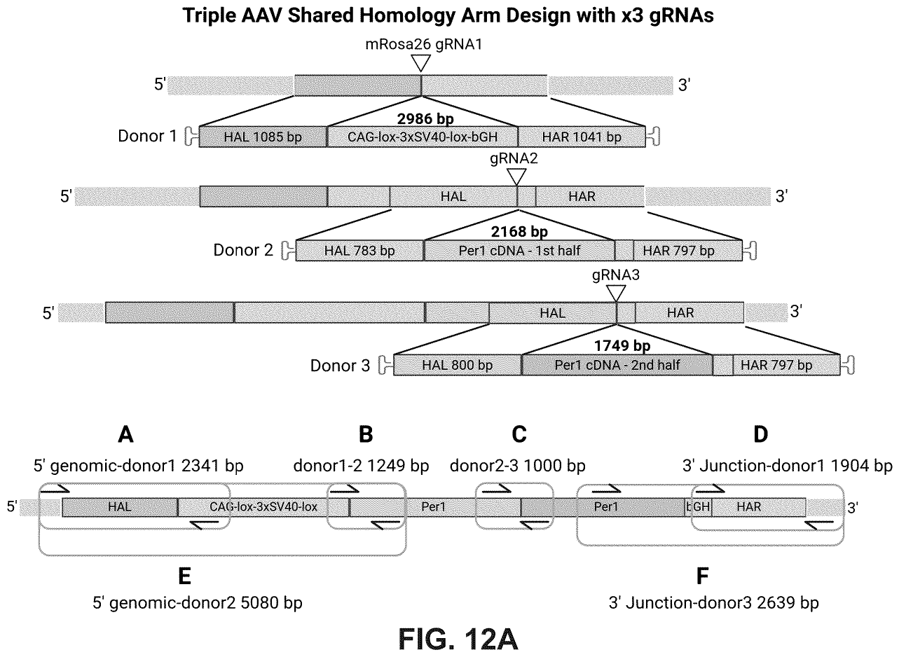

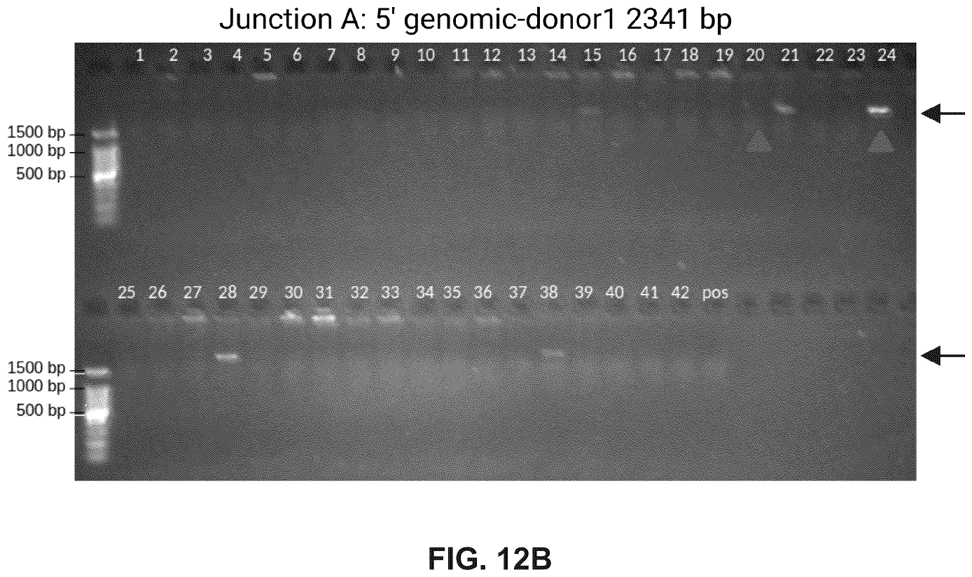

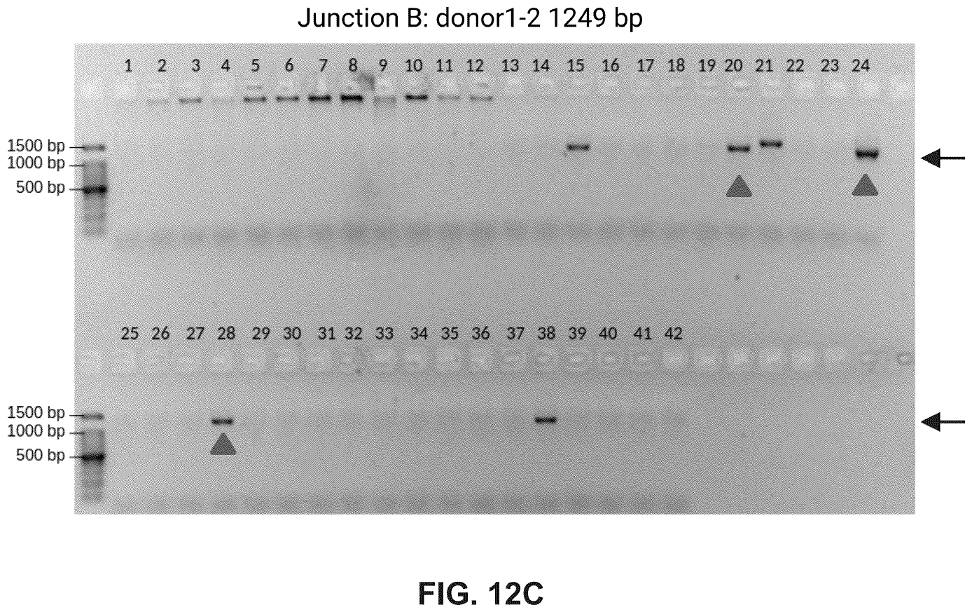

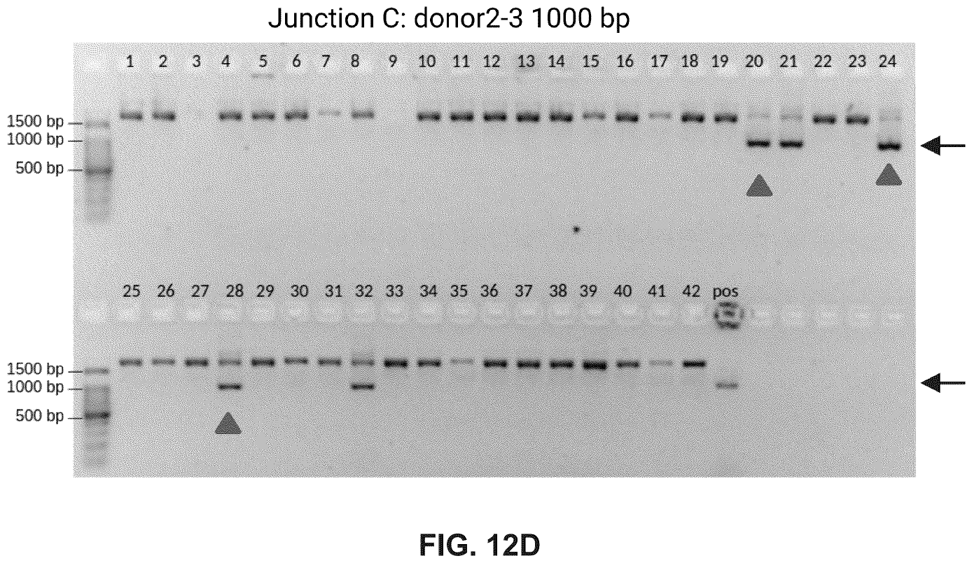

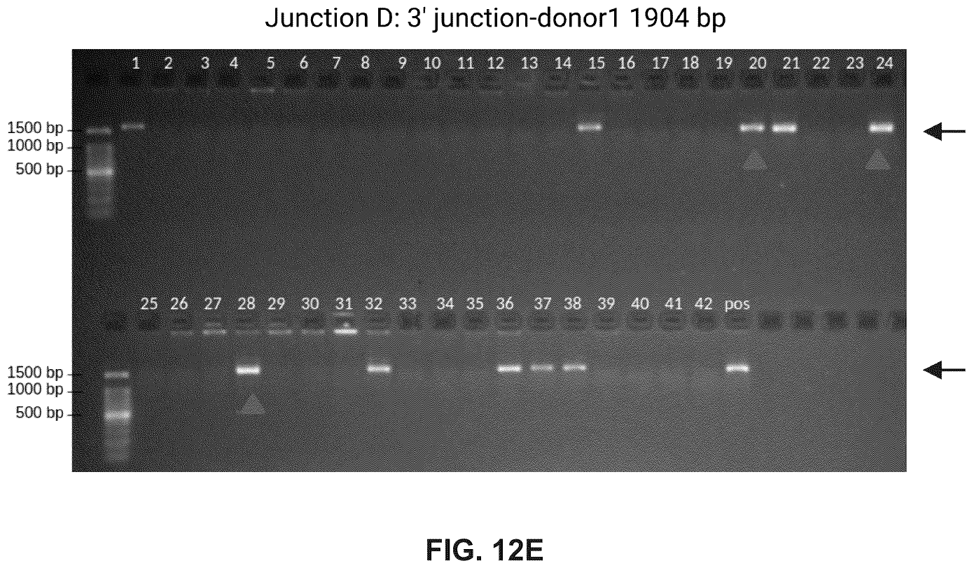

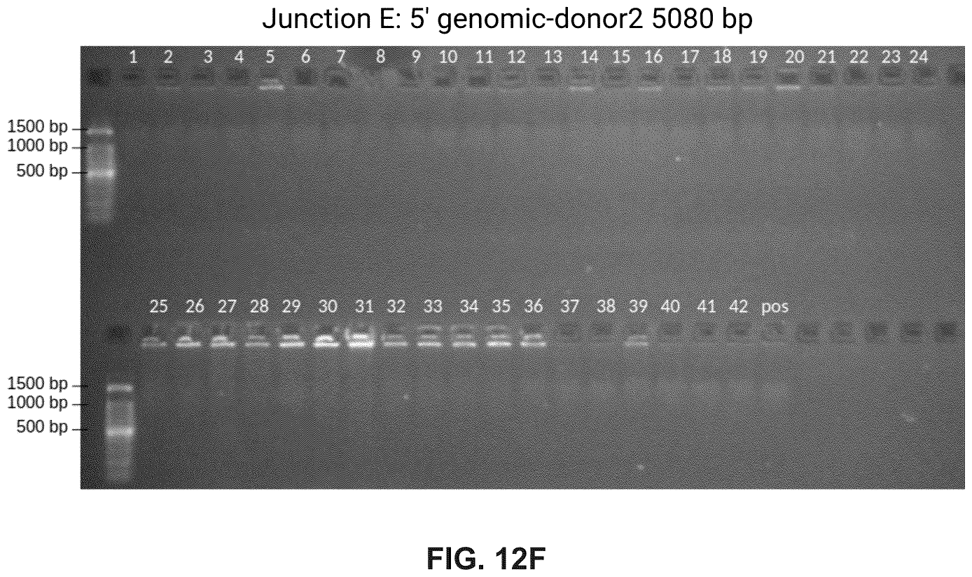

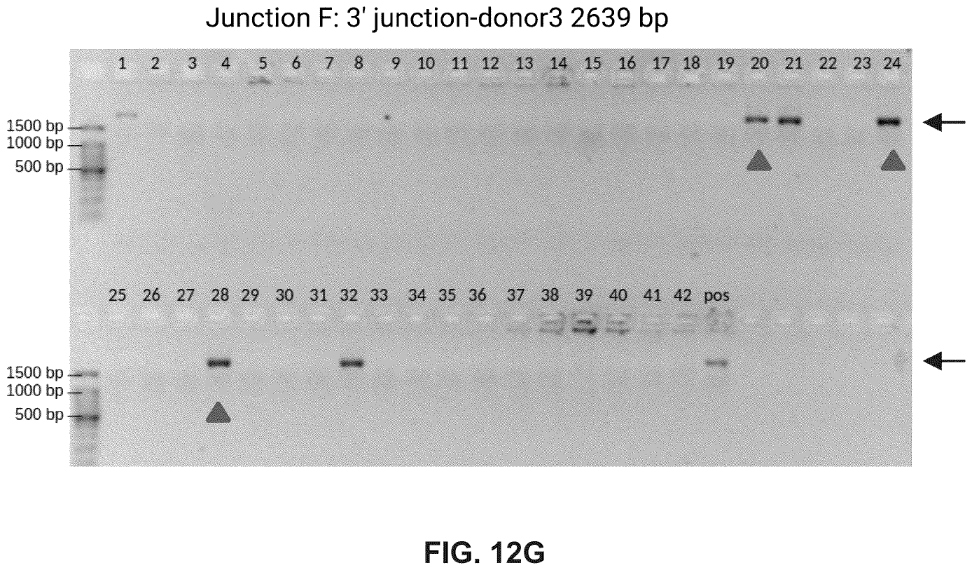

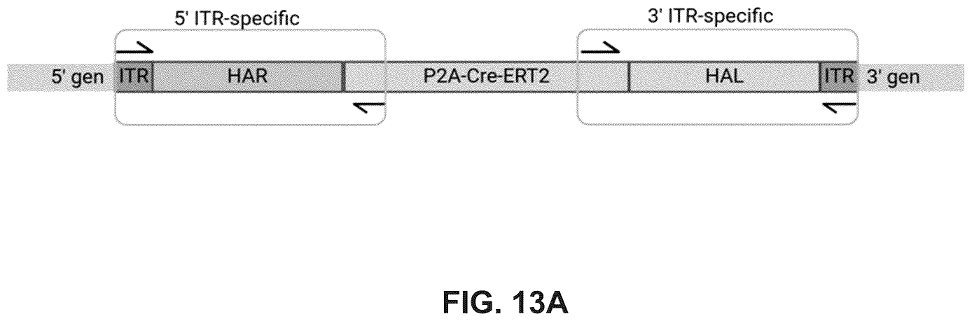

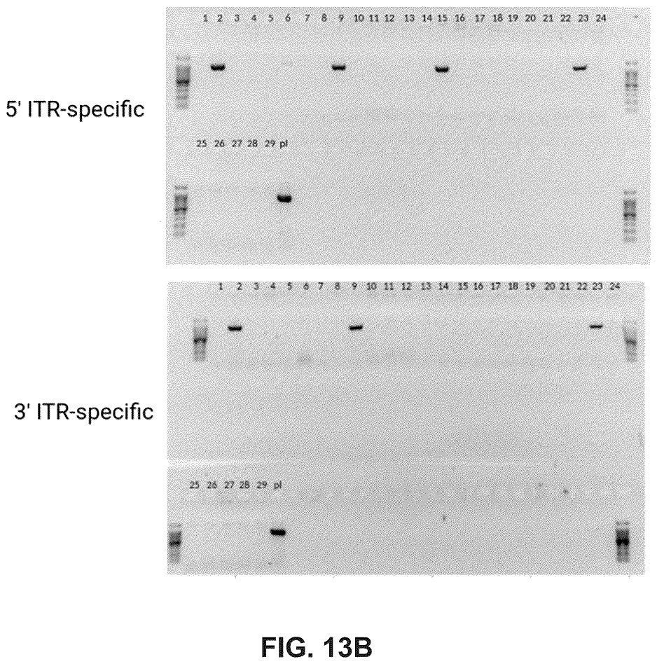

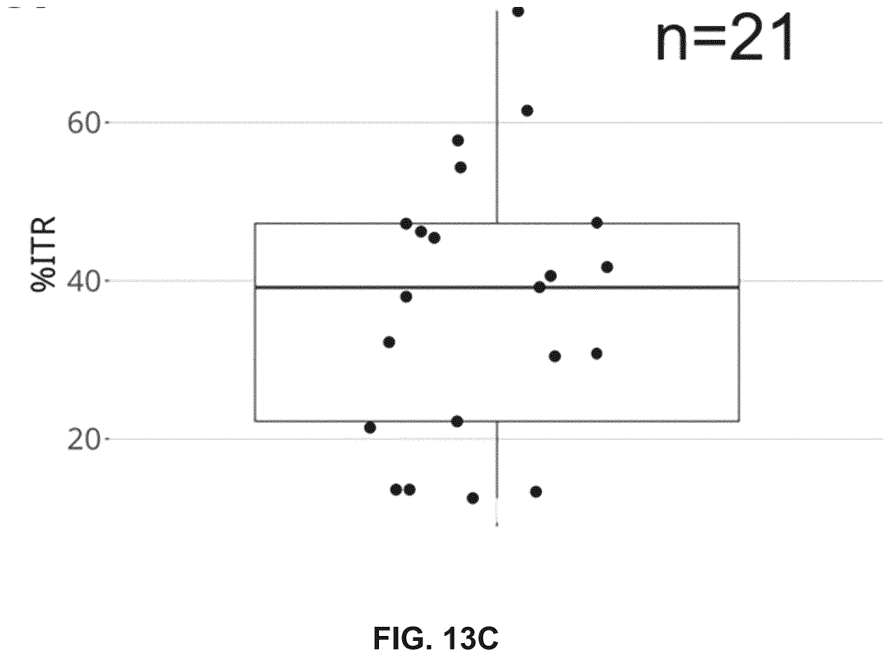

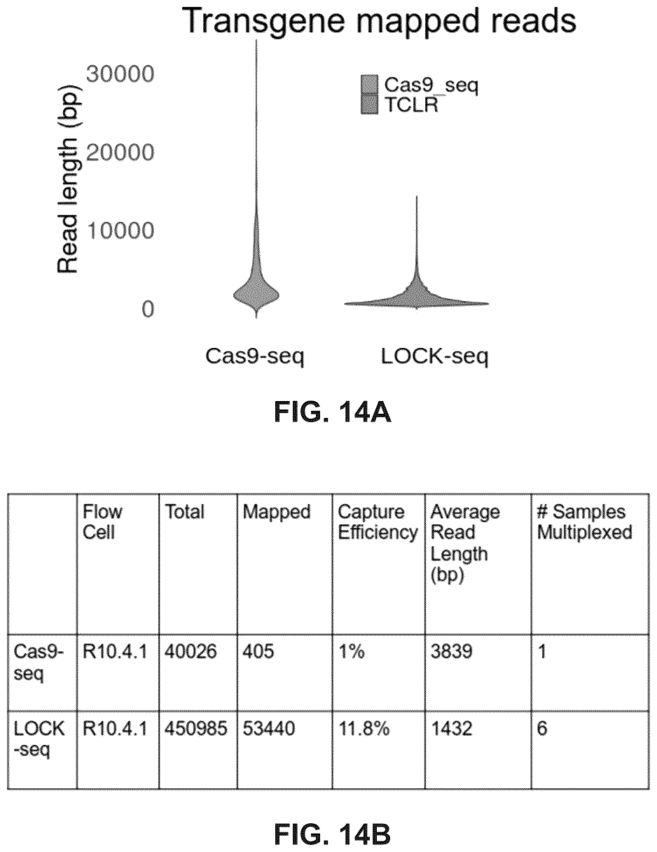

The increasing insert size via sequential HDR events renders genotyping by junction PCRs tedious and inconclusive for several reasons, including low-complexity sequences, mosaicism for the KI allele in founders, and imprecise insert. Multiple PCRs are required to screen founders, targeting the internal cassette and genomic junctions, yet results can be difficult to interpret when animals are positive for only a subset of PCRs (FIG. 11(A-E)). Adding to the complexity, founder animals are often mosaic and harbor more than two alleles. The presence of positive junctions do not guarantee a full-length insertion event. And the correctly targeted allele, if much less abundant, can be missed in PCR detection. The use of multiple rAAV donors further complicates screening, as each additional donor introduces two more new junctions (FIG. 12(A-G)). A reliable method with minimal bias is required for screening such complex events. Additionally, rAAV random integration has been reported previously. To better prioritize animals without viral genome integration, primers were designed that bind to the hairpin region of the ITRs and donor sequence so that animals negative for the ITR-specific PCR and positive forjunctions are prioritized (FIG. 13(A-C)). On average, 37% of junction positive animals were also positive for ITRs. However, the PCR-based method does not reveal the random integration sites in the genome and will miss random integrations without ITRs.

### LOCK-Seq

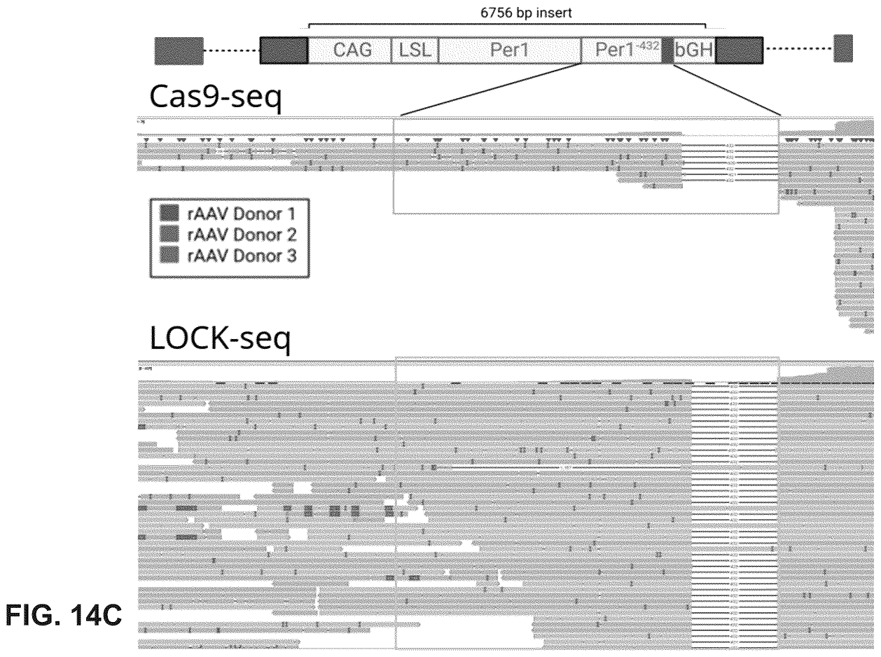

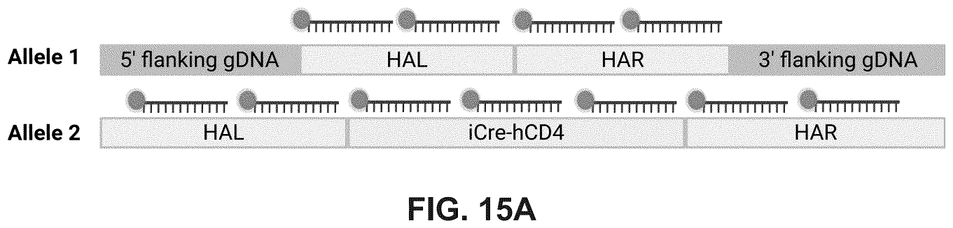

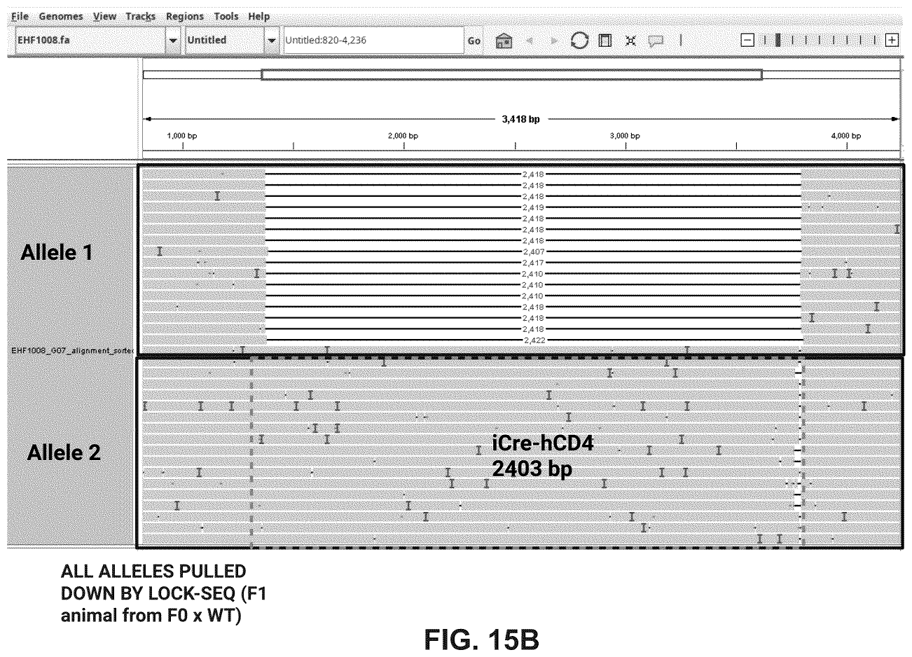

To better characterize rAAV-mediated KI events, the Cas9 Sequencing Kit (a.k.a. nCATS) from Oxford Nanopore (SQK-CS9109) was first used, which depends on Cas9/gRNA binding and cleavage close to the target. Although on-target reads were captured, the low coverage and throughput make it impractical for screening multiple founders (FIG. 14(A-C)). A new method with significantly higher efficiency of target enrichment than using Cas9/CRISPR recognition is needed.

A probe-based approach was developed using 5′ biotinylated 120-mer DNA oligos complementary to the insert, homology arms, flanking genomic regions, and ITRs and it was termed LOCK-seq. High molecular weight genomic DNA is fragmented (by using Tn5 transposition or physical sharing with Covaris g-TUBEs), ligated to adapters with barcodes, and undergoes low-cycle PCR amplification to enrich adapter-ligated product. Barcoded samples corresponding to the same target are pooled and hybridized to the capture probes for selective pull-down of sequences of interest.

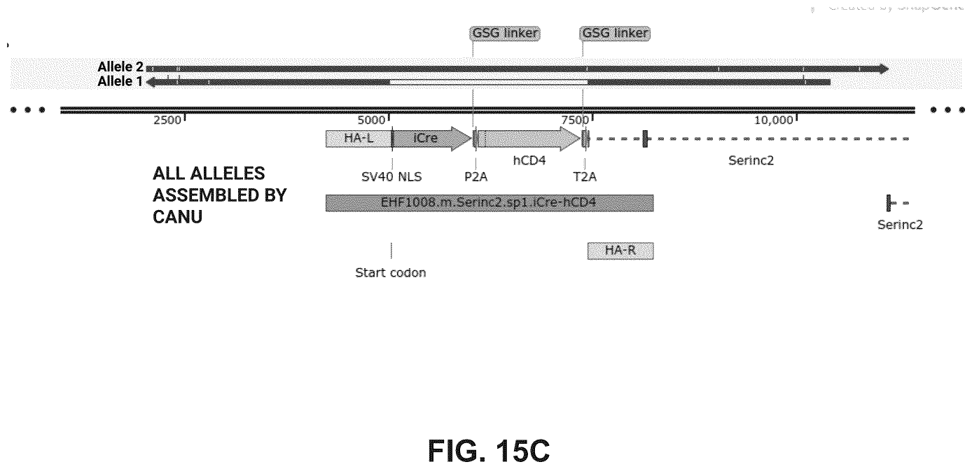

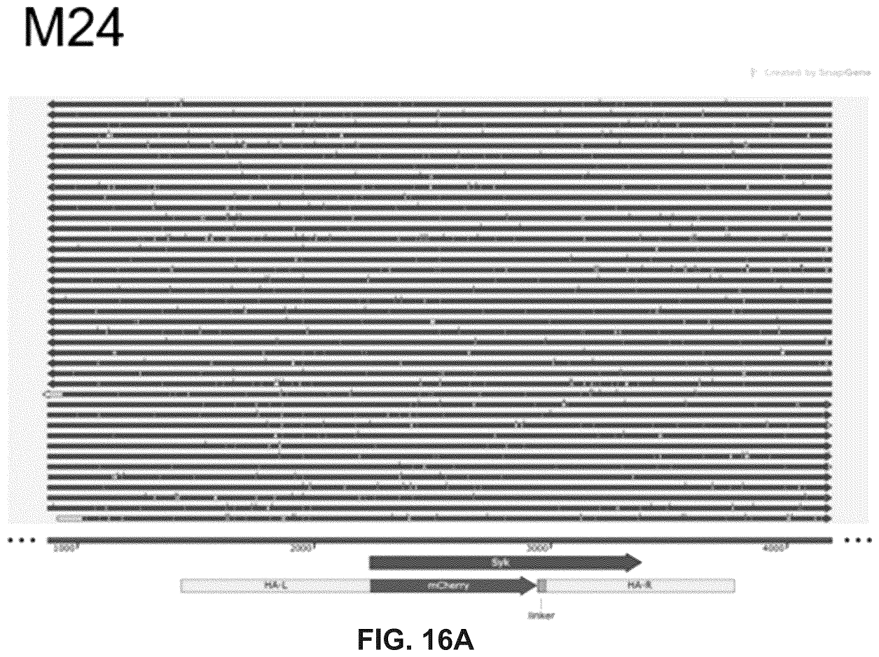

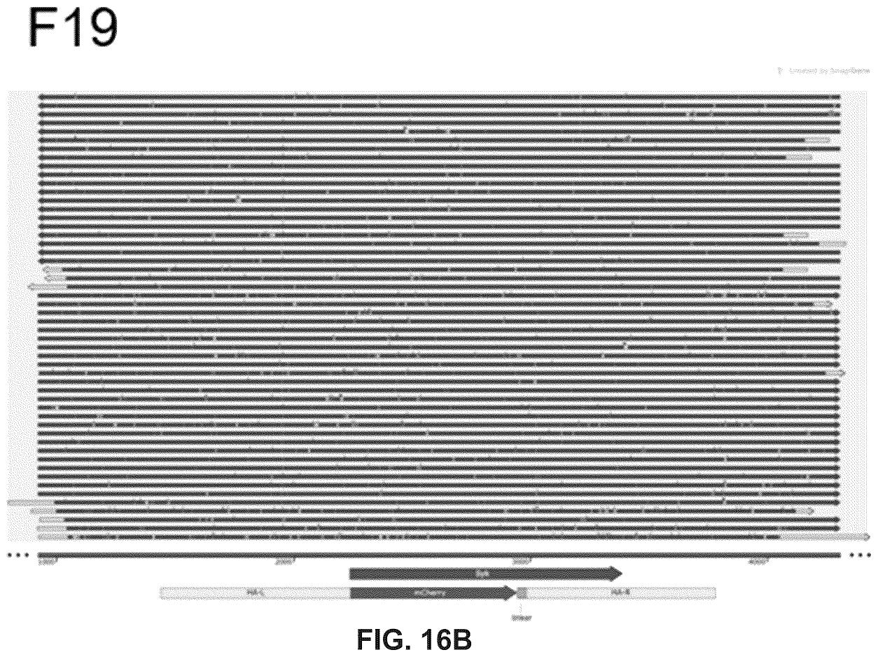

In a direct side-by-side comparison, LOCK-seq yielded >100-fold on-target coverage than nCATS (FIG. 14(A-C)). This is attributable to two factors. First, low on-target capture efficiency (1% vs 10%) for nCATS and excess adapter-free DNA that although cannot productively occupy pores, can transiently interact with pores and motor-protein bound DNA, reducing pore occupancy. The increased enrichment and read depth open the door to multiplexing many barcoded samples on a single MinION flow cell, making large-scale genotyping of founders feasible (FIG. 15(A-C) and FIG. 4A).

Moreover, LOCK-seq requires as little as 250 ng of genomic DNA, compared to 1-5 mg for nCATS, which is a significant amount to isolate from routine tissue biopsies of founder animals. Importantly, LOCK-seq identifies both knock-in alleles and indels at the target site, including random donor integrations, even if donor(s) only partially integrated elsewhere in the genome.

Four mouse samples (a founder, M24, and three F1s of M24: F19, F20 and F21) were first analyzed with a 741 bp mCherry cassette inserted at the Syk gene, one of the 108 mouse models were created using a single rAAV (data not shown). The genomic DNA was fragmented using Covaris g-TUBEs. Biotinylated 120-mer single stranded oligo probes tiled the entire length of the 2.4 kb rAAV genome and 2 kb of flanking sequences. The samples were pooled and underwent a double hybridization reaction where the enriched product from the first found of hybridization underwent a second hybridization with fresh probes to maximize specificity. The post-hybridization, single-stranded DNA was then PCR amplified, ligated to motor protein and run on a PromethION flow cell (8× the number of pores compared to MinION flow cell), given previous poor depth obtained using nCATS.

The average length for mapped reads was 2.7 kb across these four samples, with the maximum read-lengths ranging from 84-102 kb, average on-target coverage of 254K reads/sample and resulting in a capture efficiency of 85% (data not shown). Full-length, continuous reads spanning the donor and flanking genomic DNA outside of both homology arms were recovered for all samples (FIG. 16(A-E)). Out of the four samples, F20 was closest to a heterozygous insertion with equivalent coverage across the insert, supporting the absence of aberrant integration events (FIG. 4B). The other two F1 samples, F19 and F21, shared similar patterns in coverage variation as the founder, indicative of random integration events that were further analyzed below. It was determined that the combination of using a PromethION flow cell and double hybridization was overkill even for multiplexed samples. MinION flow cells and a single hybridization step were used on later samples.

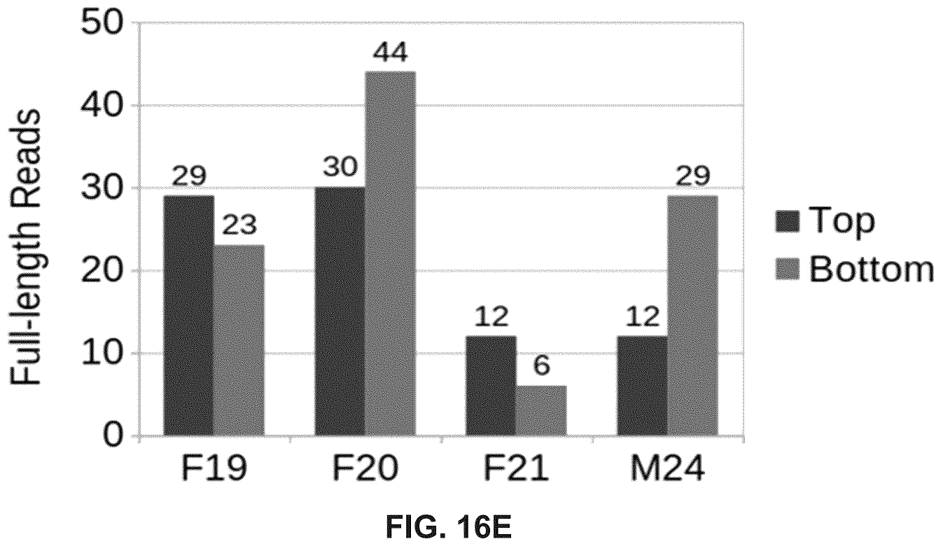

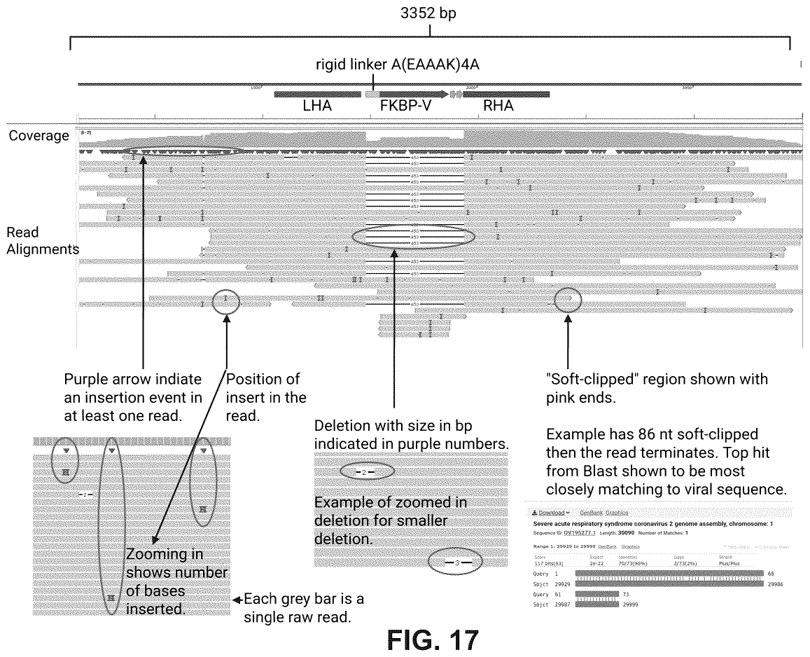

FIG. 4C shows an IGV screenshot of raw aligned reads (bam files) used to generate coverage plots and identify deviations from expected read depth consistent with copy number variants of a FKBP-V insertion (Project MS2757). The data identifies the founder as with on-target integration and without donor random integration (FIG. 17 and FIG. 18).

Three related models generated by sequential insertions from three rAAV donors were then analyzed for conditional expression of a full-length Per cDNA or the same Per cDNA with 54 bp and 432 bp deletions, respectively. For each model, only the third donor is unique. The longest insert of the series is 6.7 kb, and the founder rate was 2-7% HDR (Project XCC113, data not shown). With this set of samples, an alternative fragmentation method using Tn5 was tested, called tagmentation, to simultaneously fragmentate and barcode the DNA samples for convenient multiplexing. An average read length of 1 kb across samples was obtained, with maximum read lengths between 10-14 kb. The capture efficiency was 11-19%, with an on-target sequencing depth range across samples of 6K to 38K reads (data not shown). The 54 bp and 432 bp deletions are evident in respective samples (FIG. 4D). An increase in coverage for a portion of the wild type Per cassette implies concatenation of donor 3 (one of three donors used). Further analyses are shown below. Compared to Covaris g-TUBEs, enzymatic tagmentation is convenient and high throughput, with the downside of shorter fragment size, hence short read lengths. However, it is a useful method for quickly screening tens of samples.

### Various Undesired Modifications Detected Only by LOCK-Seq

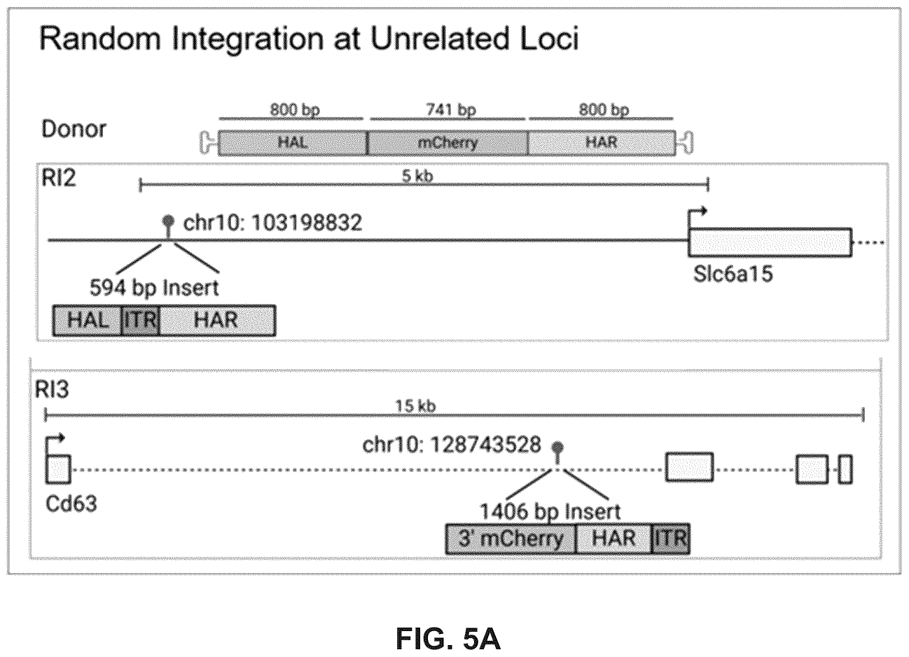

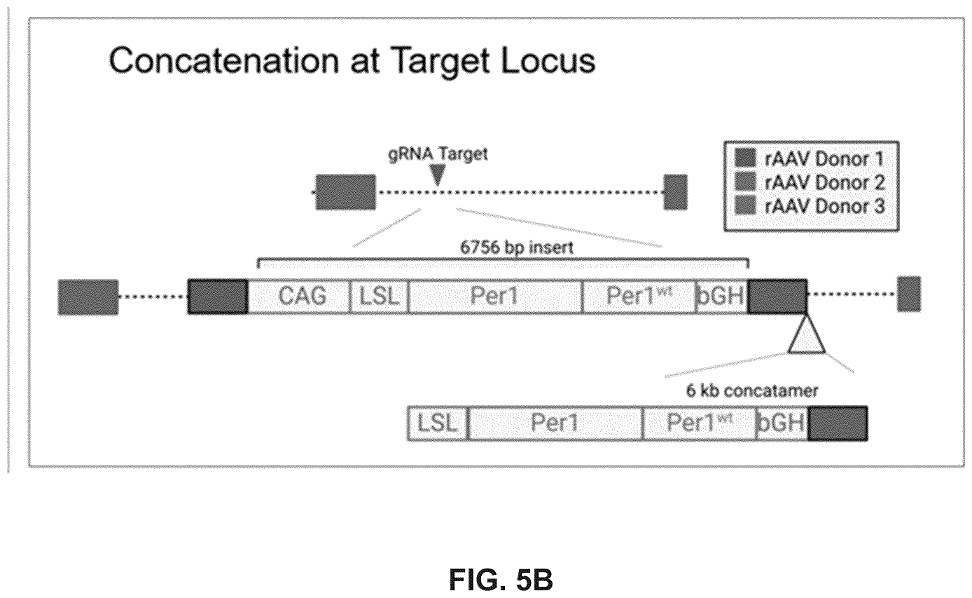

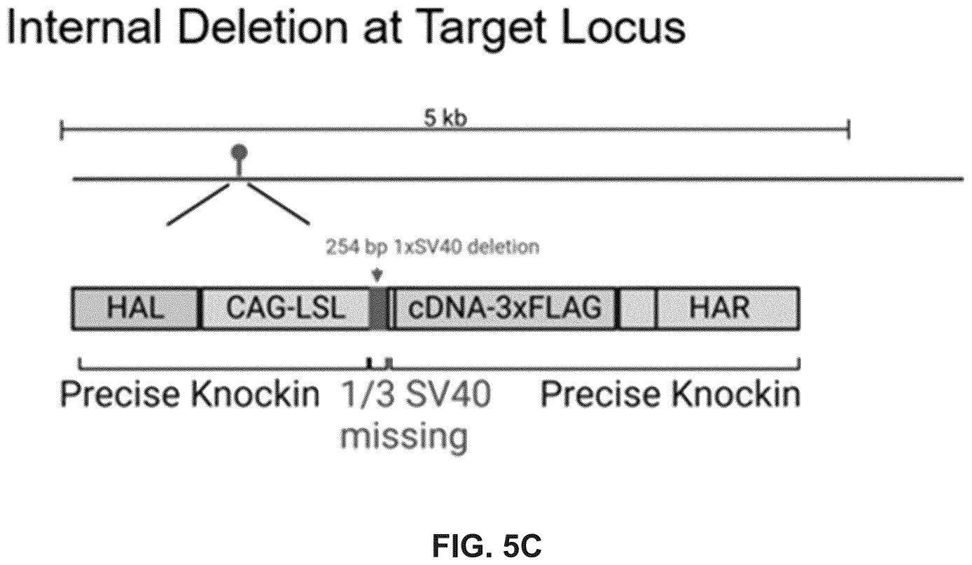

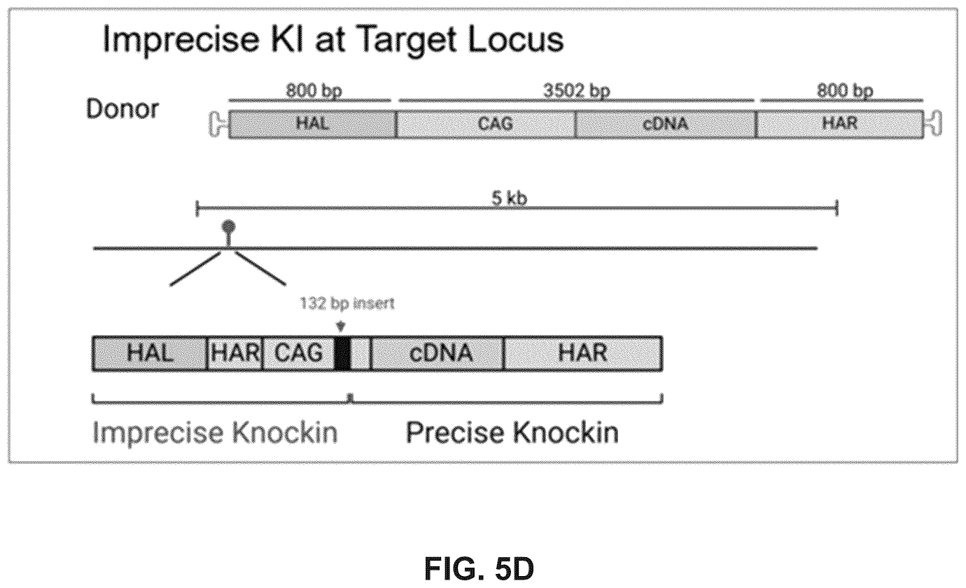

Undesired edits among the samples at or away from the target sites were next analyzed more closely. The per-base read depth for M24, F19 and F21 suggests partial integration of the 3′ of mCherry and homology arms (FIG. 4B). Alignments of mapped reads to the mm39 mouse genome revealed a random integration event within the first intron of Cd63 (chr10: 128,743,596, mm39) and 5 kb upstream of neurotransmitter transporter Slc6a15 (chr10: 103,198,832) for M24 that were transmitted to F19 and F21, but not F20 (FIG. 5A). Both random integrations were confirmed by site-specific PCRs of the insertion junctions (FIG. 19(A-B)). A third random integration at a transcript with unknown function, 49305000H12Rik (chr16:73,129,317), was detected in Founder M24 but not identified in its progeny.

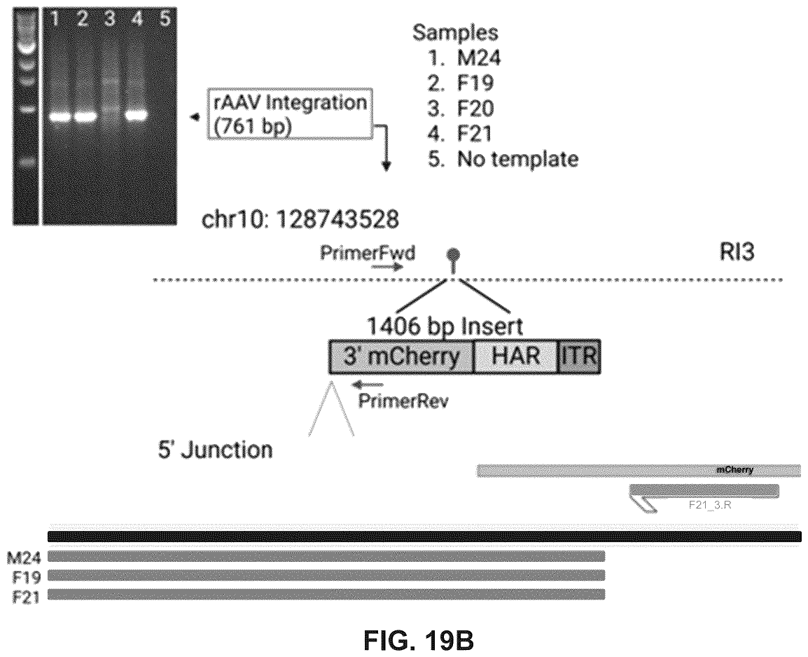

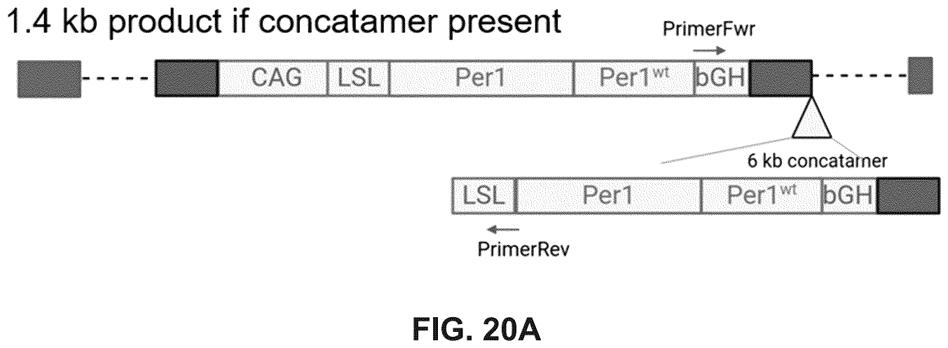

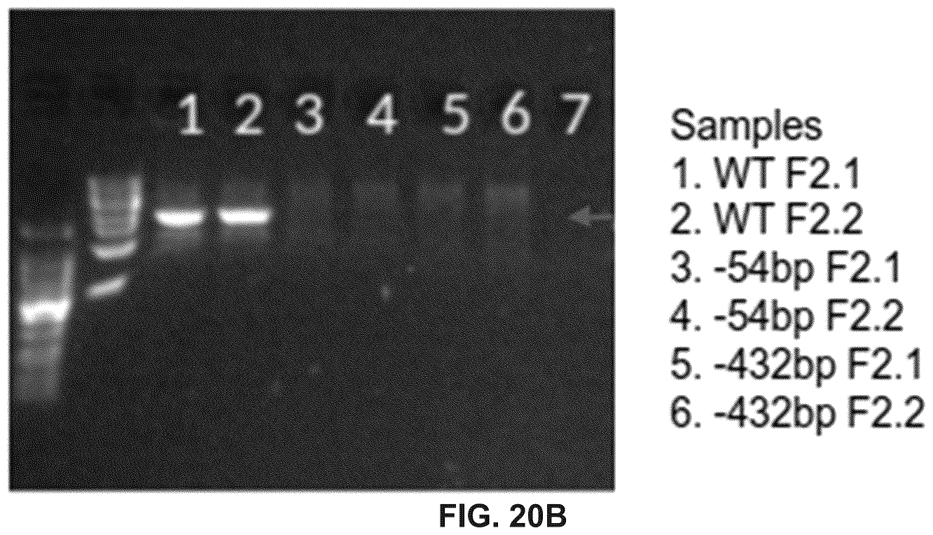

Of the three Per cDNA x3 rAAV models (Project XCC113), the variation in coverage across the cDNA cassette in the WT model proved to be a concatenated insertion event at the target site that was evident after de novo assembly of reads, despite the shorter average read length using Tn5 tagmentation (FIG. 5B). The concatenation was confirmed to be present only in the WT model by PCR (FIG. 20(A-C)). Besides concatenation, frequent internal deletions of one of the three SV40 polyA signal were also observed when screening 3-rAAV founders (FIG. 5C), which would be missed by junction PCRs. One-sided imprecise KIs were also observed, as in the case of a 3.5 kb reporter at the ROSA locus (Project MS2750) with insertions that could impact transgene expression (FIG. 5D).

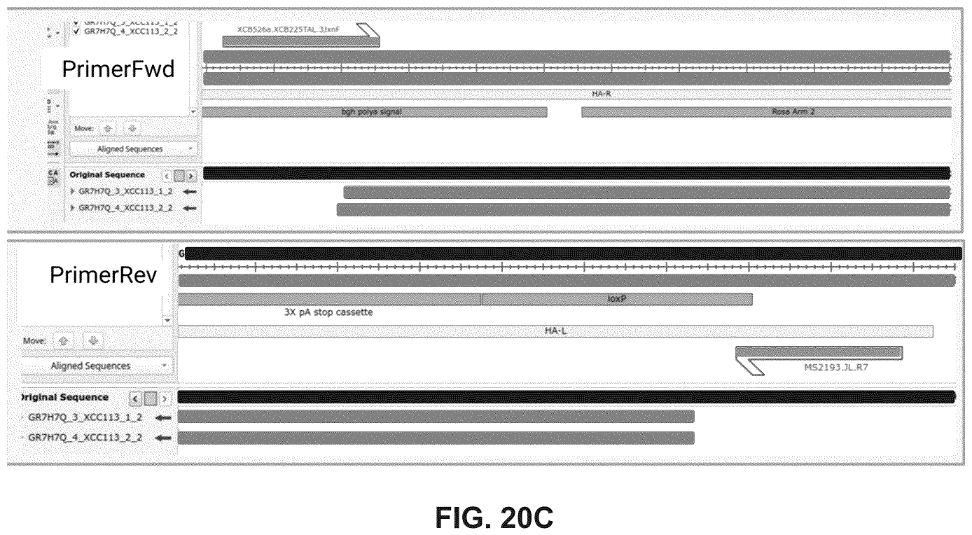

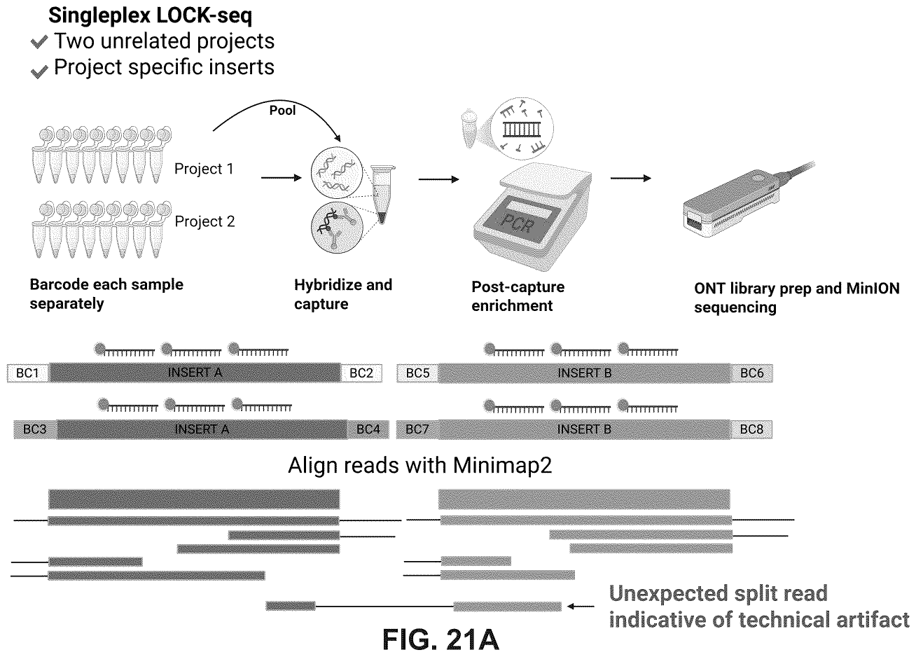

The possible generation of technical artifacts, such as fusions reads that could falsely signal random integration of the donor, weas also explored. Such artifacts have previously been reported to occur in ˜2% of ONT data generated from amplicon sequencing, despite each amplicon being generated in single-plex reactions. In this case, a single run consisting of samples from different projects, where each project has unique sequence elements knocked-in should not contain any fusion reads between the two unique sequence elements. Two MinION runs were analyzed, each with multiple samples from two different projects that were carried through the LOCK-seq protocol that included fragmentation, barcoding, hybridization, ONT library prep, and ONT sequencing. For each MinION run analyzed, fusion or split reads where one read aligned to both unique insertion events were counted across samples (SEQ ID NO: 1003-1006). <2% fusion artifacts were found (FIG. 21(A-B)).

### Flexible and Cost-Effective Probe Generation

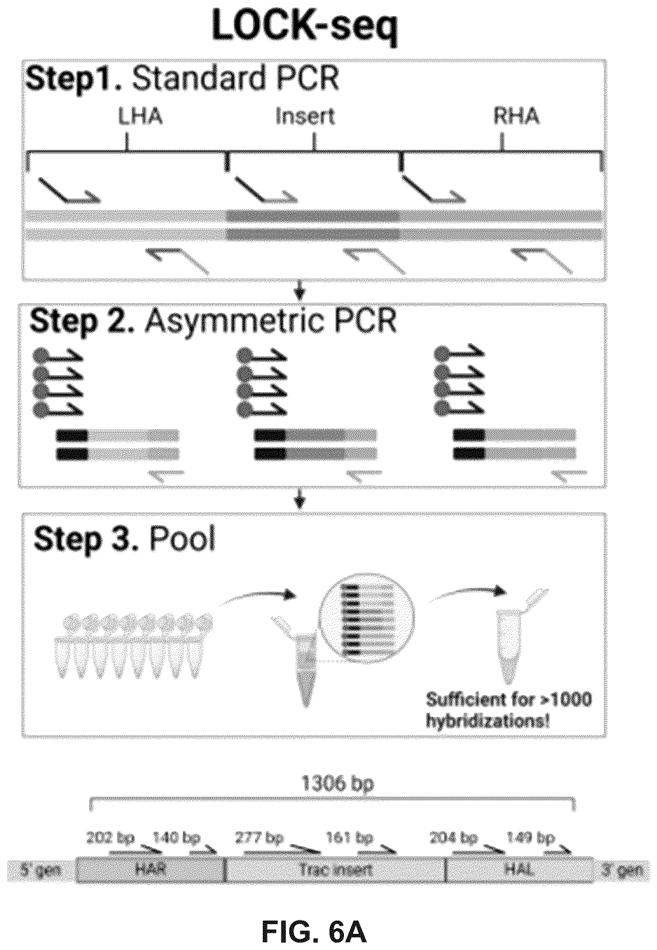

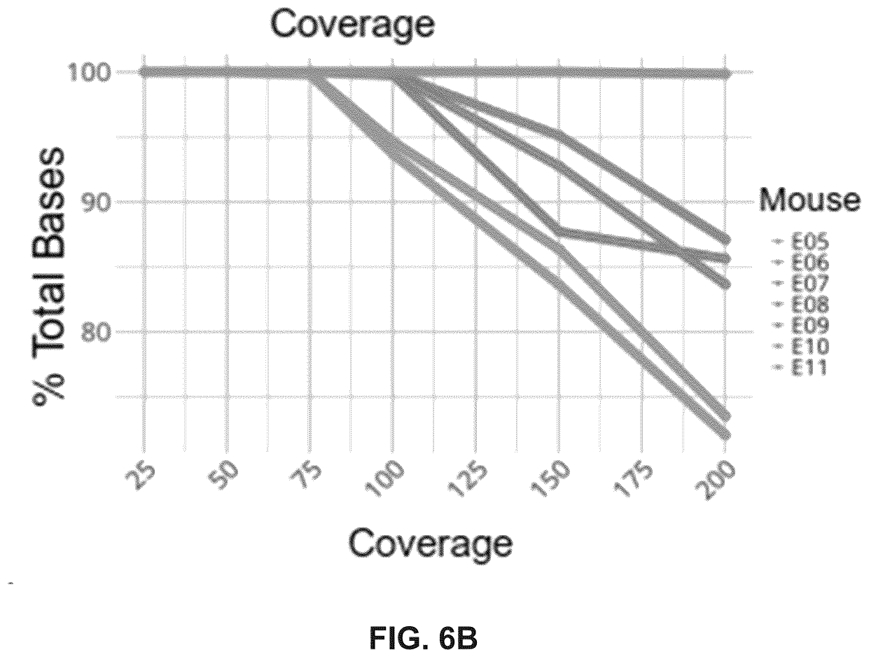

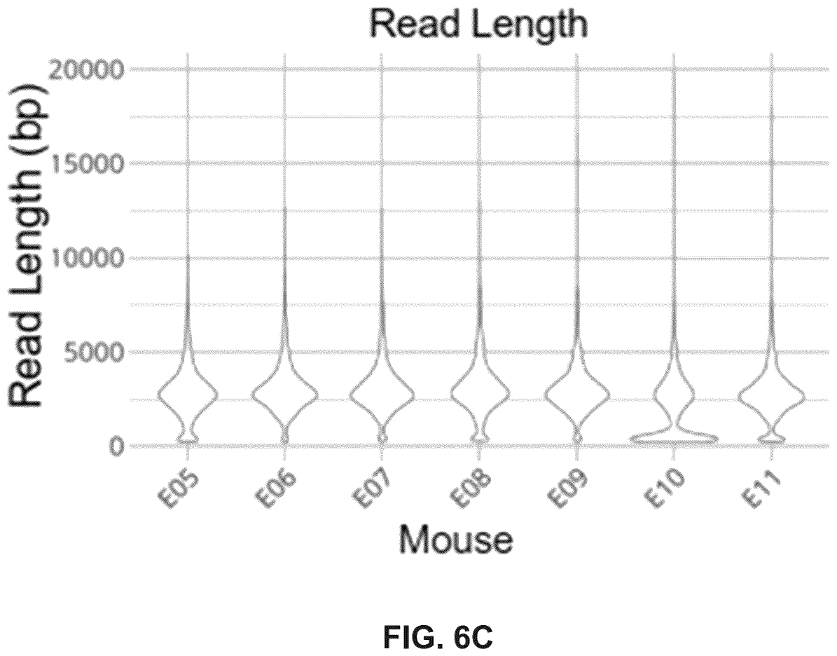

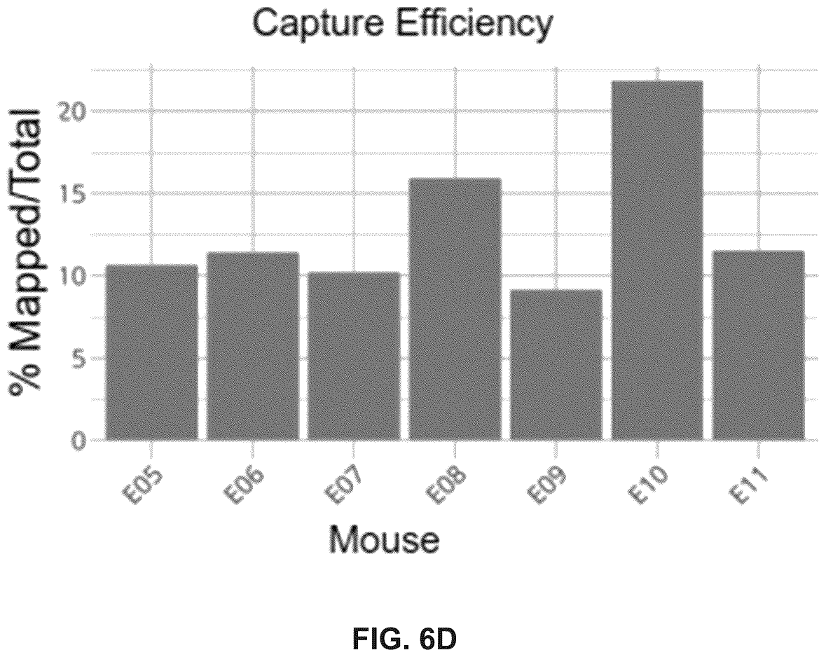

The biotinylated 120-mer oligo probes are costly, especially when used to te across the genomic region. To help reduce cost, amplicons spaced ˜300 bp apart along the genomic region and primers with universal tails were designed to create probe templates for a second, asymmetric PCR with biotinylated primer that anneals to the universal tail, resulting in low density tiling across the region of interest (FIG. 6A). The pooled and cleaned up asymmetric PCR reactions are now a probe set sufficient for hundreds of hybridizations. As shown at the bottom of FIG. 6A, four probes ranging from 140-270 bp were tiled across 1.3 kb region with 300 bp or greater spacing for LOCK-seq on samples for Project MS2826 (data not shown). Good uniformity of coverage across all samples was obtained, achieving 55× coverage for 99% of bases and x coverage for >90% (FIG. 6(B-E)). Using this small panel, average read lengths were 2-4 kb across samples with a range of 2.5-17K mapped reads and capture efficiency of 10-20%. Given that a MinION (R10.4.1) flow cell outputs 30-50 Gb of data, which averages about M total 5-10 kb reads per run, one could multiplex 100 samples to achieve about 1000 reads per sample at a 10% capture efficiency.

TABLE 1
LOCK-seq using PCR generated probes significantly reduce cost while facilitating design flexibility. Comparison
of probe cost with nCATS (synthetic gRNA and Cas9 protein RNPs, use if 2-4 RNPs per target locus) with
LOCK-seq probes that are commercially synthesized or generated in-house through asymmetric PCR.

| Feature | nCATS (Cas9 RNP-based) | LOCK-seq (Probe-based capture) |
|---|---|---|
| Capture Mechanism | Cas9/gRNA RNPs bind and | Biotinylated DNA probes hybridize to target; captured |
| | cleave dephosphorylated | fragments amplified via adapter-specific primers |
| | genomic DNA to release | |
| | regions of interest | |
| Sequencing Platform | ONT long-read | ONT long-read |
| Multiplexing Capacity | Low (1-3 samples per | High (≤100 samples on a MinION at ~1000x coverage |
| | MinION) | for 5-10 kb read lengths) |
| Versatility of Design | Constrained by PAM | High flexibility; probes can be placed anywhere (Insert, |
| | sequences; multiple pairs | HA, ITR, flanks), unconstrained by PAM |
| | of RNPs to improve | |
| | efficiency | |
| Capture Efficiency | ~1% | ≥10%, enables deep coverage at target locus |
| Number of Reactions | ~20 reactions (2 nmol | Commercial panels: ~16 captures Asymmetric PCR in- |
| | gRNA stock) | house: hundreds of captures possible |
| DNA Input Requirement | 1-5 μg genomic DNA | ~250 ng genomic DNA |
| Cost per 20 kb Target | ~$500 (Cas9 protein + | Commercial probes: ~$1000 (120-mer biotinylated |
| Locus | synthetic gRNAs; 4 RNPs | ssDNA panel, ~120 probes tiled at 30 bp spacing) |
| | per locus) | Asymmetric PCR in-house: ~$200 (average 250 bp |
| | | amplicons, ~70 probes tiled at 30 bp spacing) |

Compared to Cas9-based enrichment, probe capture using asymmetric PCR probes is substantially more cost-effective and scalable. nCATS requires custom gRNAs and Cas9 RNPs for each locus, typically limited to 2-4 RNPs per site, which constrains coverage, results in low capture efficiency, and cannot be multiplexed across many samples. In contrast, asymmetric PCR probes can be generated in-house at very low cost, with the ability to generated sufficient yields for hundreds of hybridizations. Probe length can be tuned (e.g., 120-300 nt or longer) depending on design needs, providing flexibility not available with commercial ssDNA biotinylated probes. Across five distinct donor targets using different gDNA mouse preps, the read length was highly reproducible at 4.5 kb for reads in the upper quartile as was an enhanced on-target coverage of 1×10⁴-1×10⁵ reads (data not shown). In these cases, the hundreds of reads in the upper quartile of read length exceed the length of the donor used, producing single reads that traverse the entire region of interest. This adds a high level of support that the on-target knock-in sequence is contiguous with the genomic-specific flanking sequence, and therefore not an artifact of assembly. Additionally, this high on-target coverage enables multiplexing of dozens to over 100 samples in a single run, dramatically reducing per-sample costs while increasing throughput.

rAAV Approach is Successful in Cancer and Stem Cell Lines

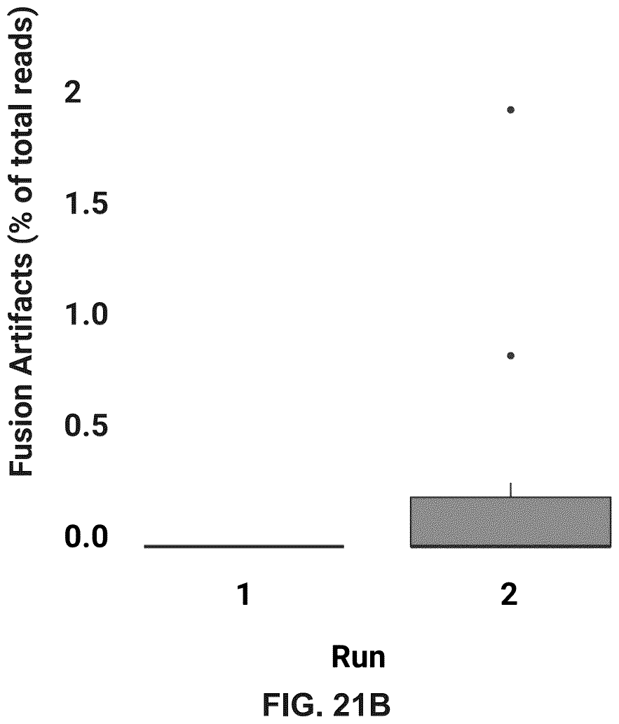

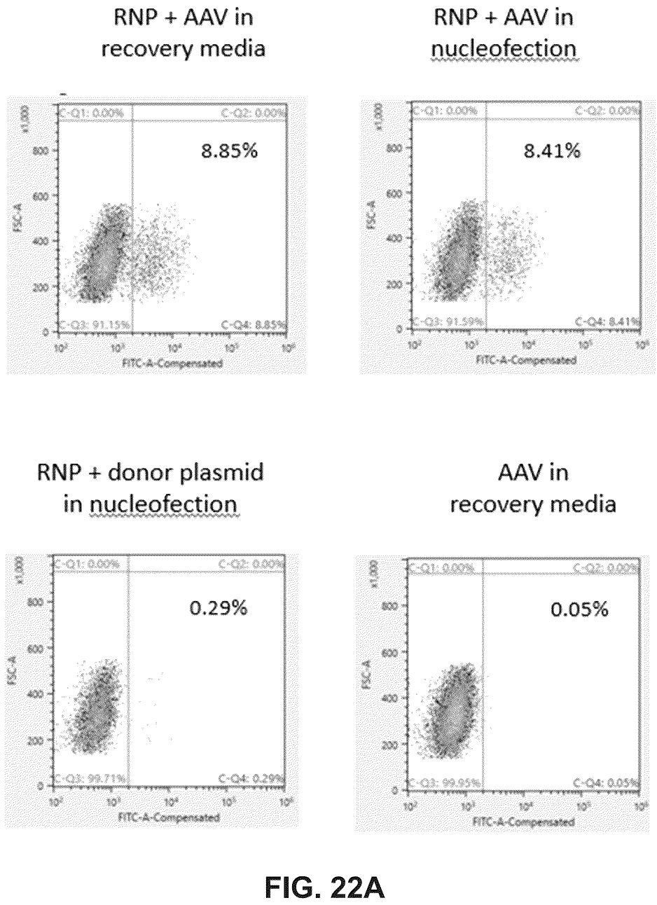

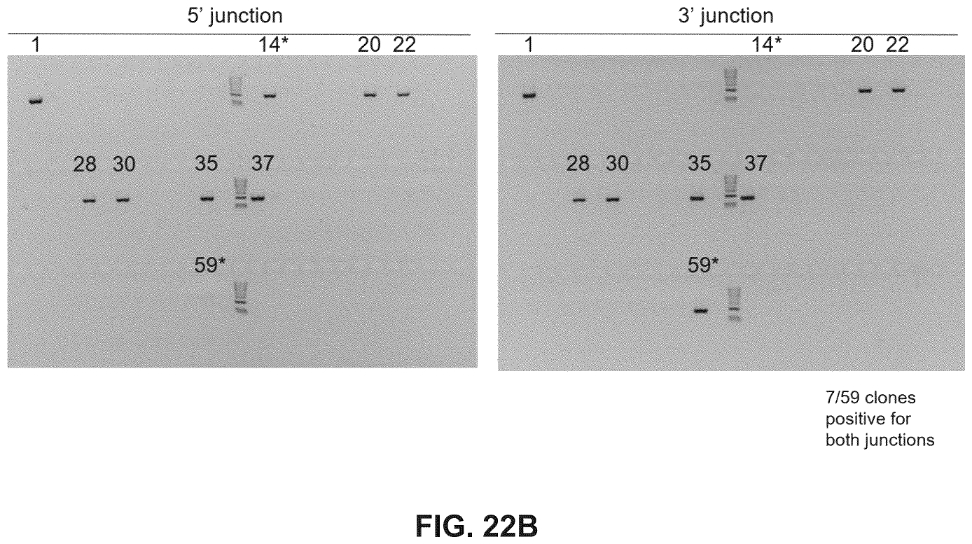

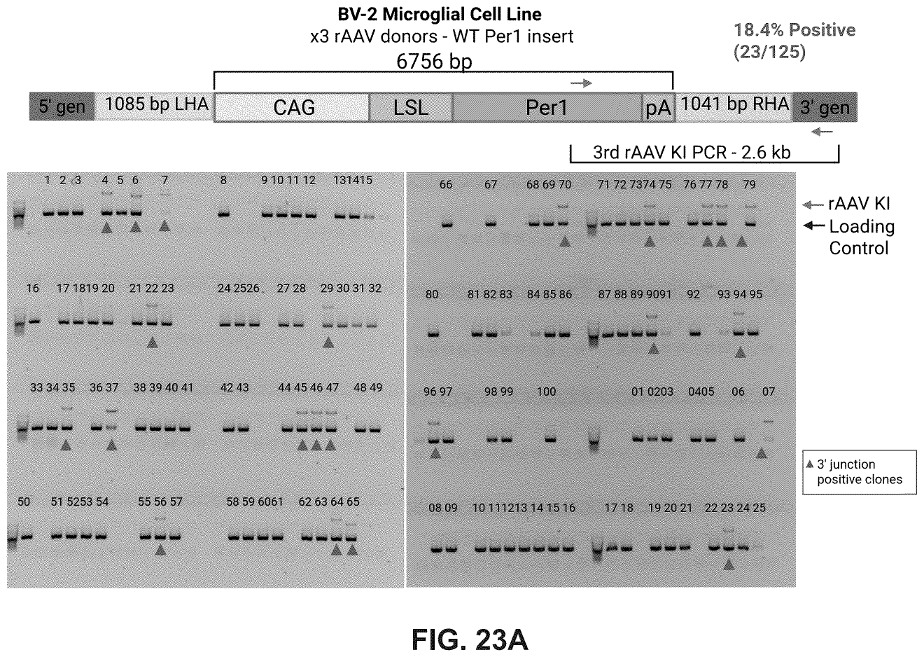

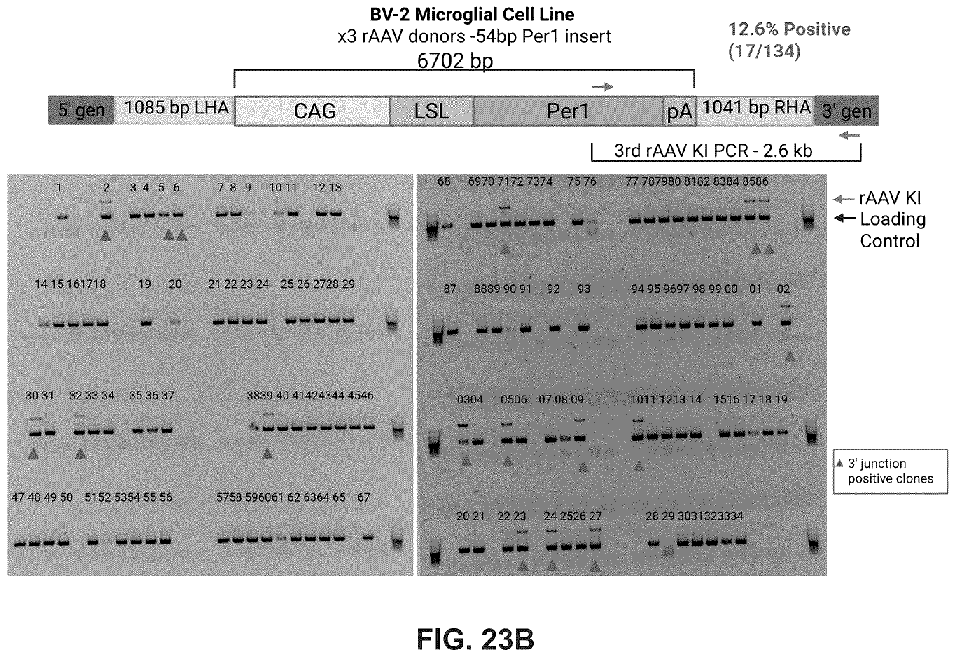

Like mouse KI models, large insertions are sometimes required for cell line models. The same strategy was applied to engineer cancer and stem cell lines, including those that are too sensitive to plasmid DNA to survive transfection, such as mouse BV2 microglial cell line. It was found that rAAV added immediately after RNP nucleofection was 30-fold more efficient than a donor plasmid for inserting a cDNA-GFP into iPSC cells, measured for GFP by FACS a week after nucleofection (FIG. 22A), and confirmed by junction PCR in single cell clones (FIG. 22B). BV2 cells are highly sensitive to exogenous DNA and show low efficiency HDR when using ssODNs. A side-by-side comparison of ssODN-mediated KI in BV2 and N2a cells showed comparable gRNA activity in both cell lines but drastically different HDR rates, from barely detectable in BV2 cells to 10-30% HDR in N2a cells (FIG. 7A). Likewise, plasmid DNA transfections often lead to nearly no survival of BV2 cells and undetectable HDR, making large cassette KI impossible to obtain. In contrast, BV2 was targeted with three 3-AAV sequential donors used for mouse projects to KI >6 kb and obtained >10% junction positive single-cell derived clones across all 3-AAV targeting projects (FIGS. 7B and 23(A-C)).

Detectable Recombination Between rAAV Donors

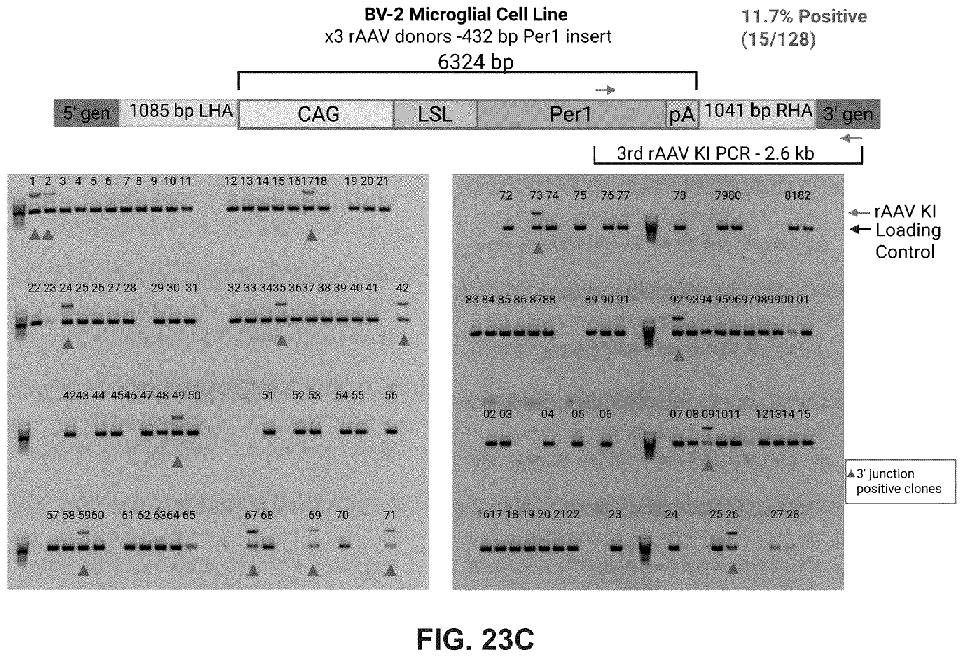

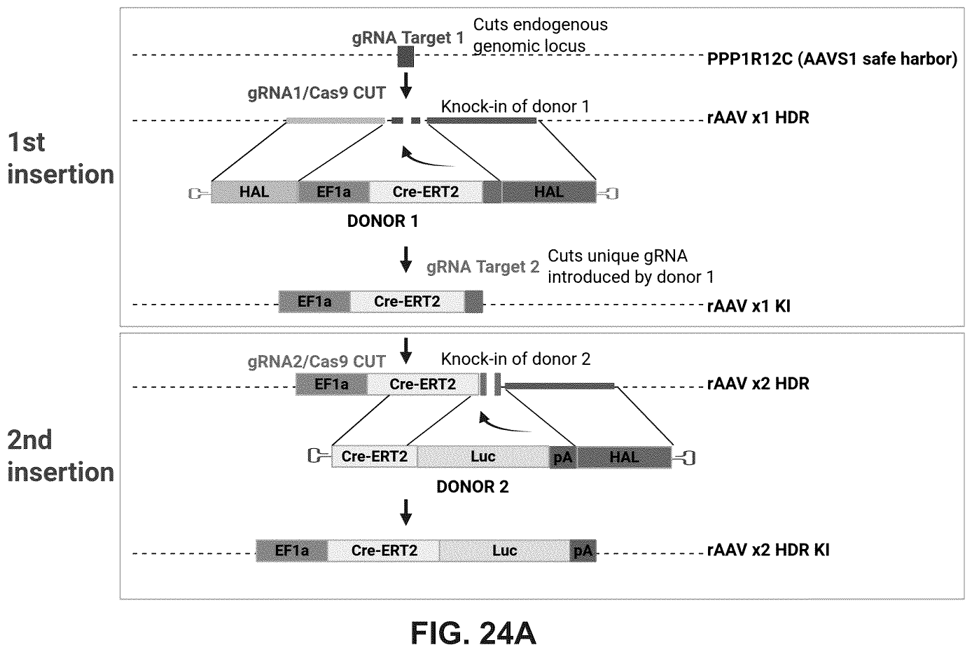

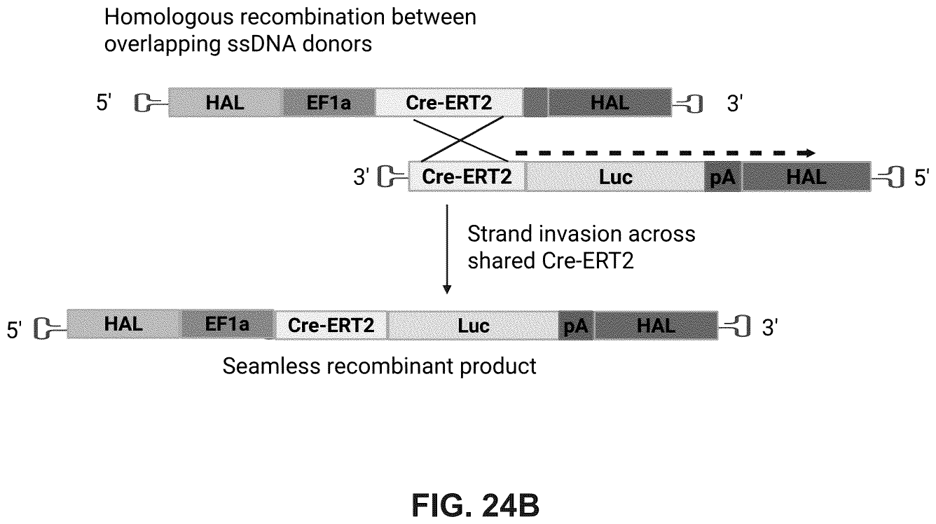

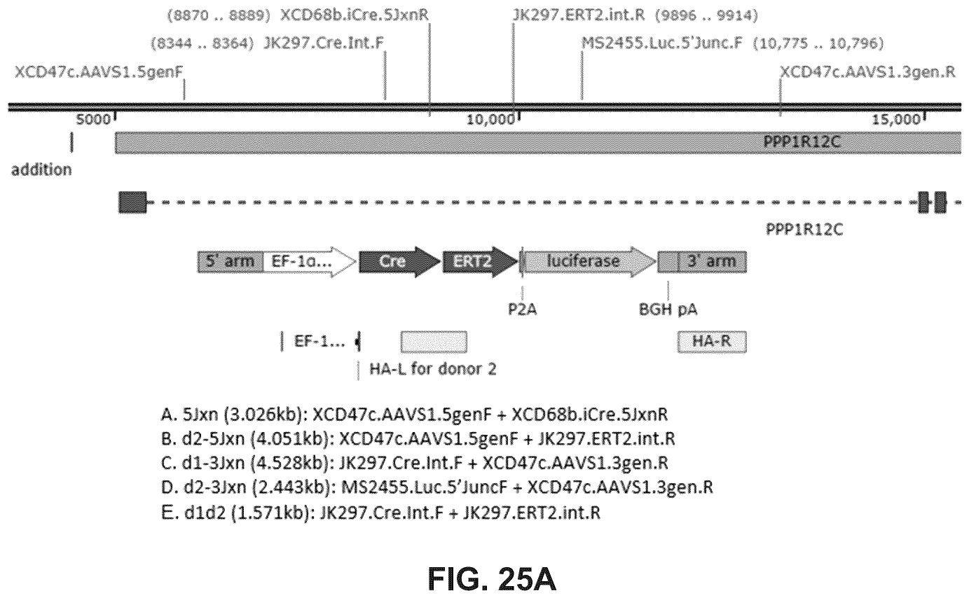

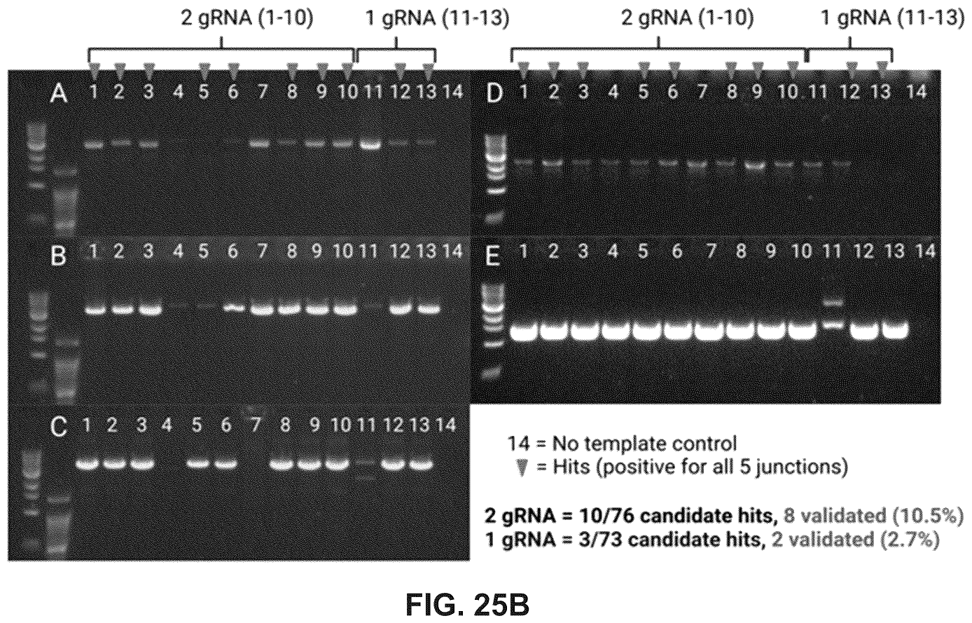

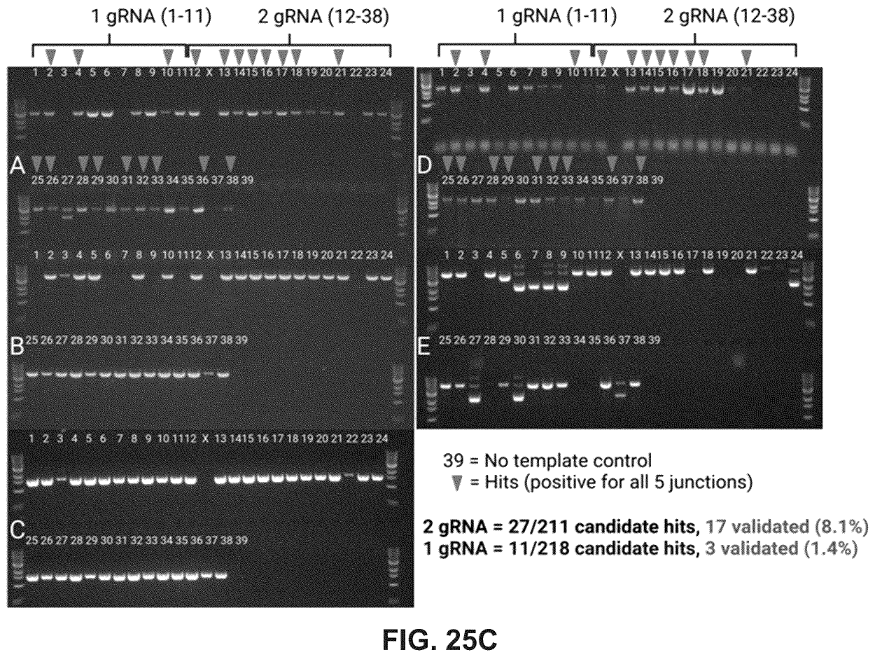

Above it was demonstrated that each double strand break generated by a Cas9/sgRNA cleavage of the chromosome mediates one HDR event, and the cleavage/HDR combination can go up to three times, achieving sequential rAAV-based KI. Single-stranded rAAV genome carrying a gRNA target site is not cleavable until KI occurs and the target site becomes double stranded, orchestrating the order of HDR events (FIG. 24(A-B)). Whether additional double-stranded breaks are needed for the second and/or third insertion events was next determined. In the absence of the second CRISPR/Cas9 complex, there is no double-strand break at the knock-in site after the first knock-in. It would require an interaction between donor 1 and 2, through inter-molecular recombination for example, to bypass the need of two independent HDR events for each half of the full-length composite sequence. Using two AAV donors, project XCD47c, to KI EF1a-Cre-ERT-T2A-luciferase-bGH pA signal, with or without the second RNP in HEK293T cells and iPSCs, was tested. Surprisingly, precise KI can occur at the target site with both donors without the need of a second double-stranded break, but much less efficiently (FIG. 25(A-D)). The percentage of junction PCR positive clones for all junctions is 3-fold higher with two gRNAs in HEK293T and 5-fold higher in iPSC cells.

### LOCK-Seq on Cell Lysates for Quick, First-Round Clonal Screen

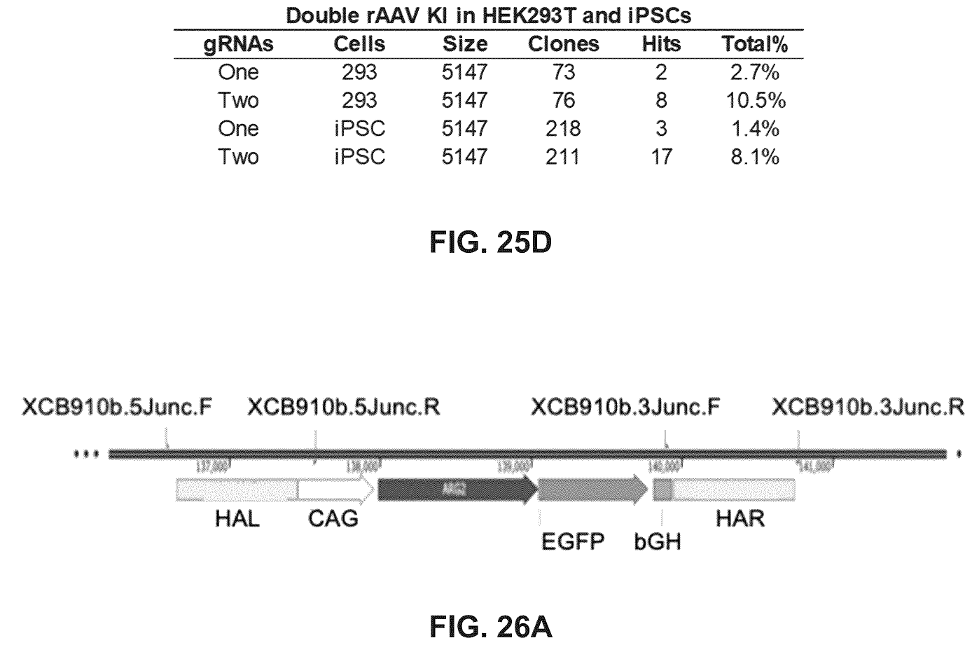

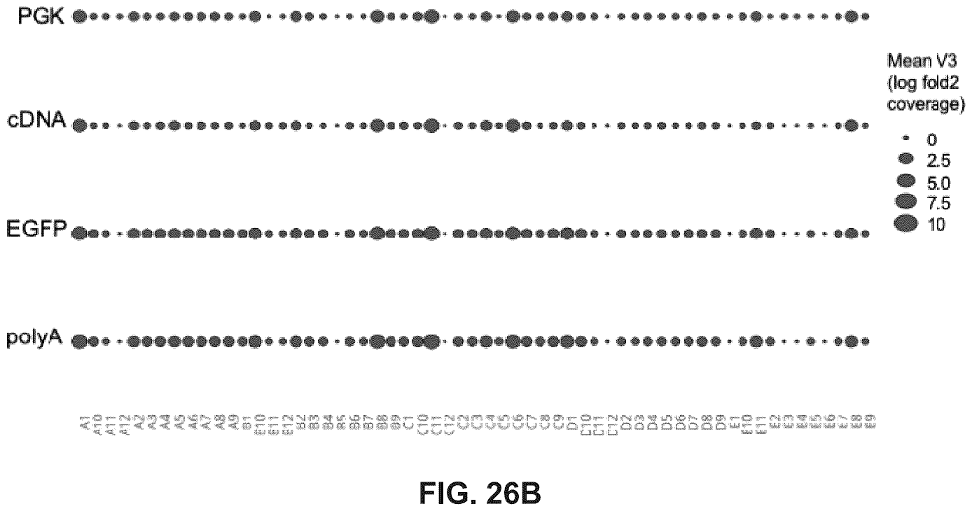

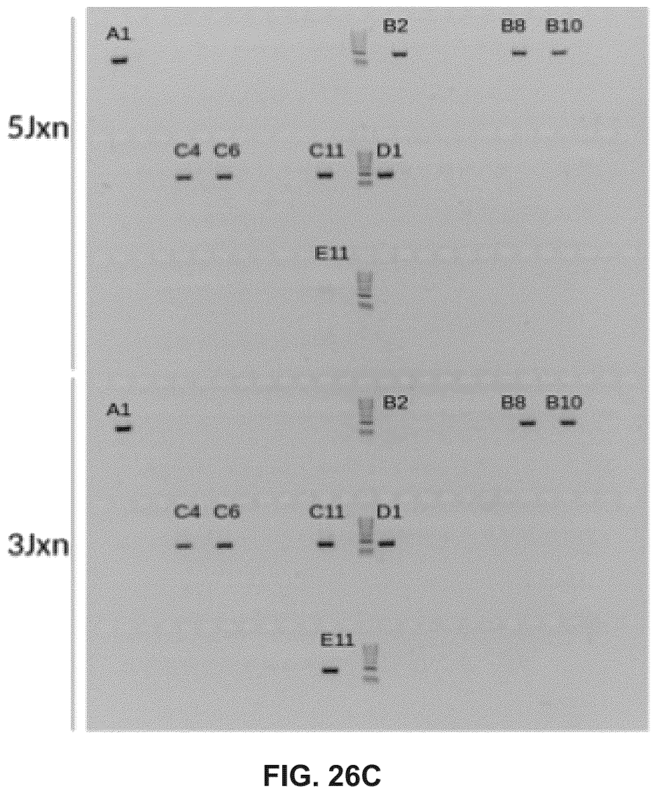

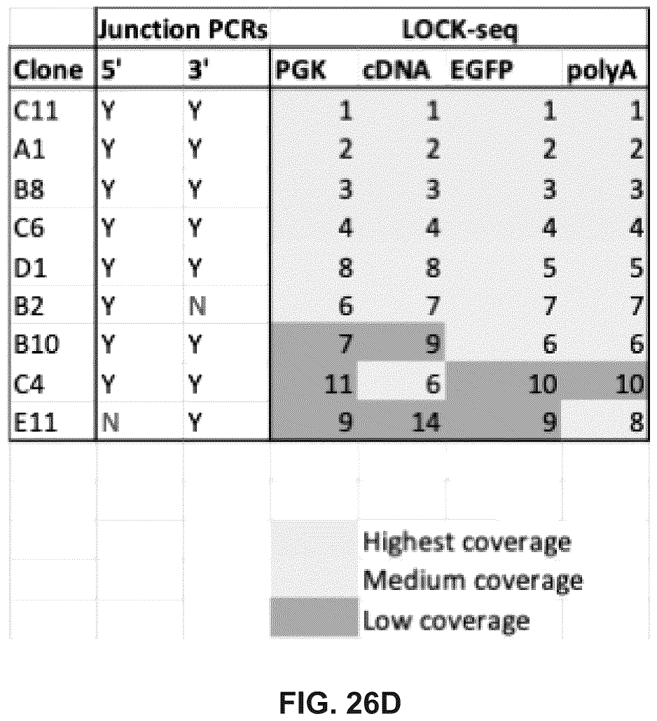

Multiplexing and using crude extract and low cell numbers were also tested, which would not be possible using the nCATS. 59 iPSC clones were screened on a single MinION flow cell using Tn5 for tagmentation of crude extract captured using commercial probes and the same nine positive hits were identified by junction PCRs, demonstrating in principle that LOCK-seq is a practical method for initial clonal screening (FIG. 26(A-D)).

### Optimization of Probe Design and Production for Better Capture Efficiency

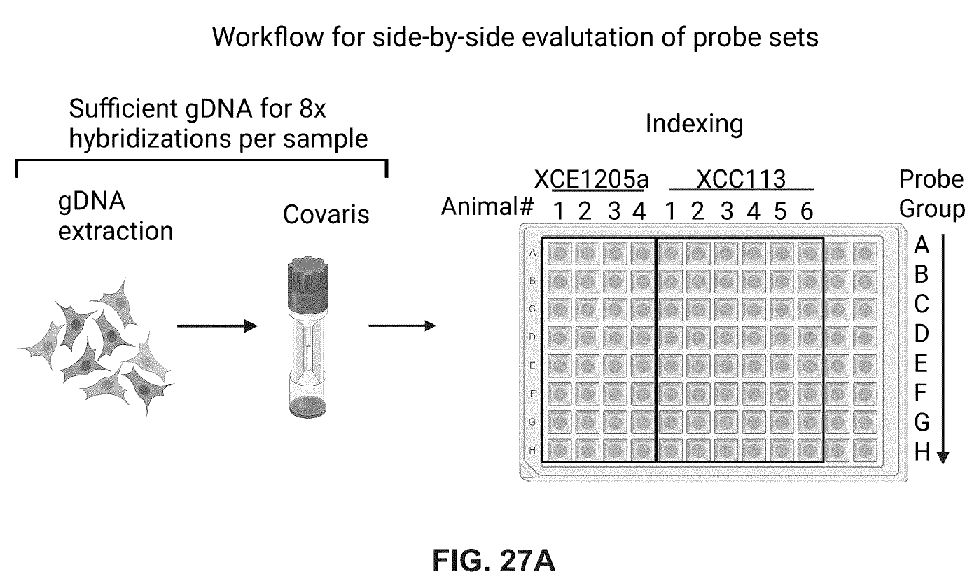

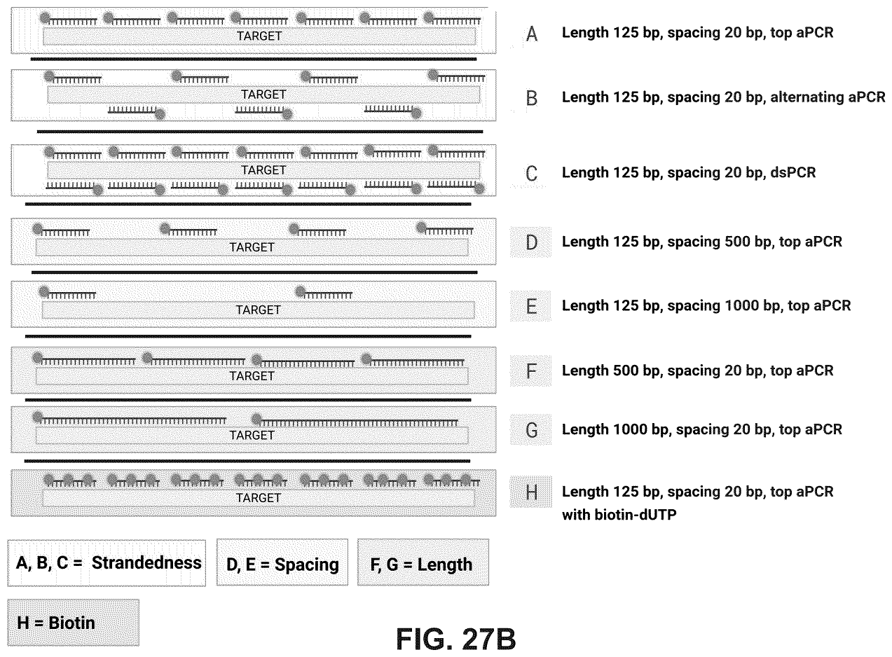

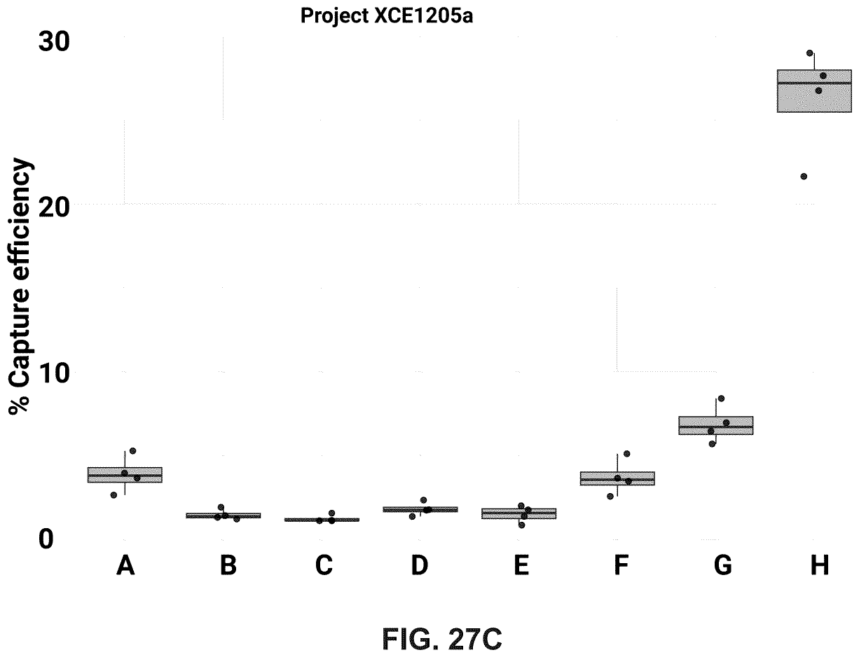

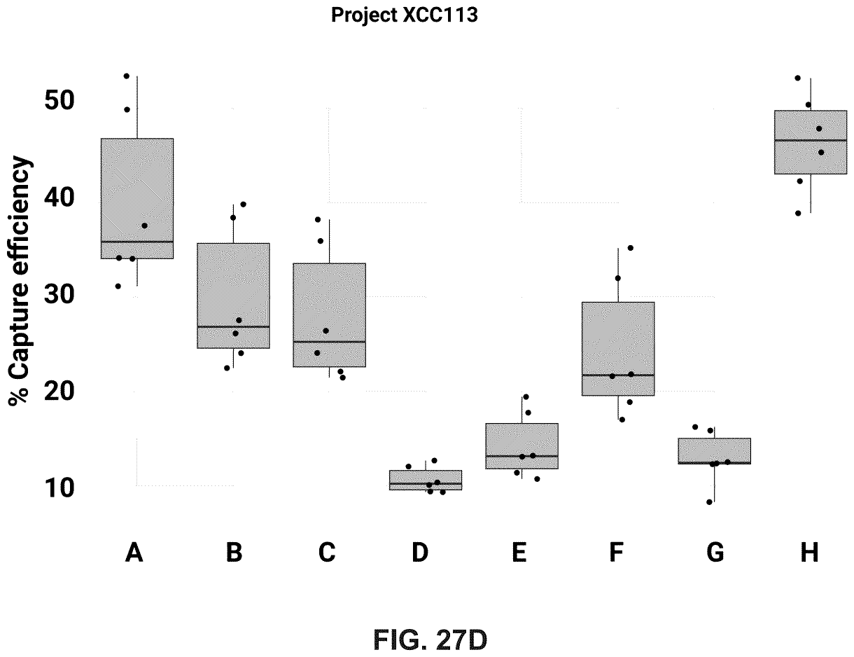

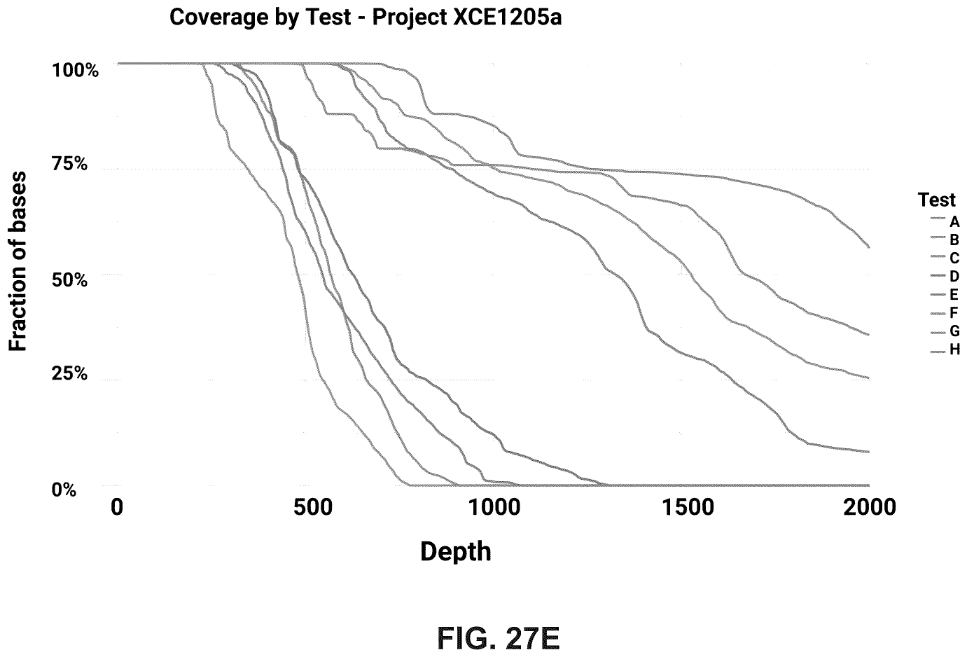

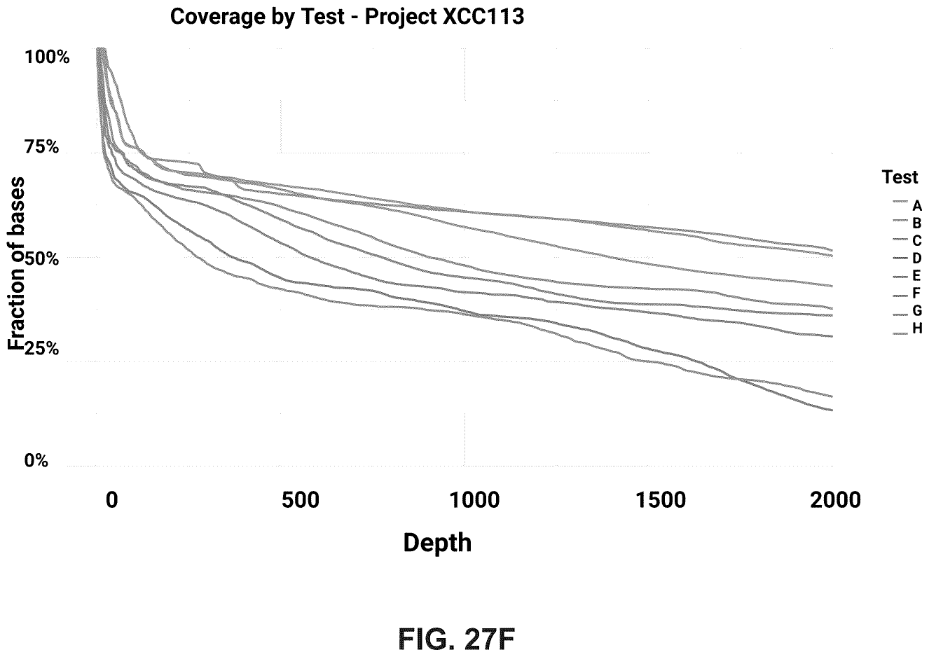

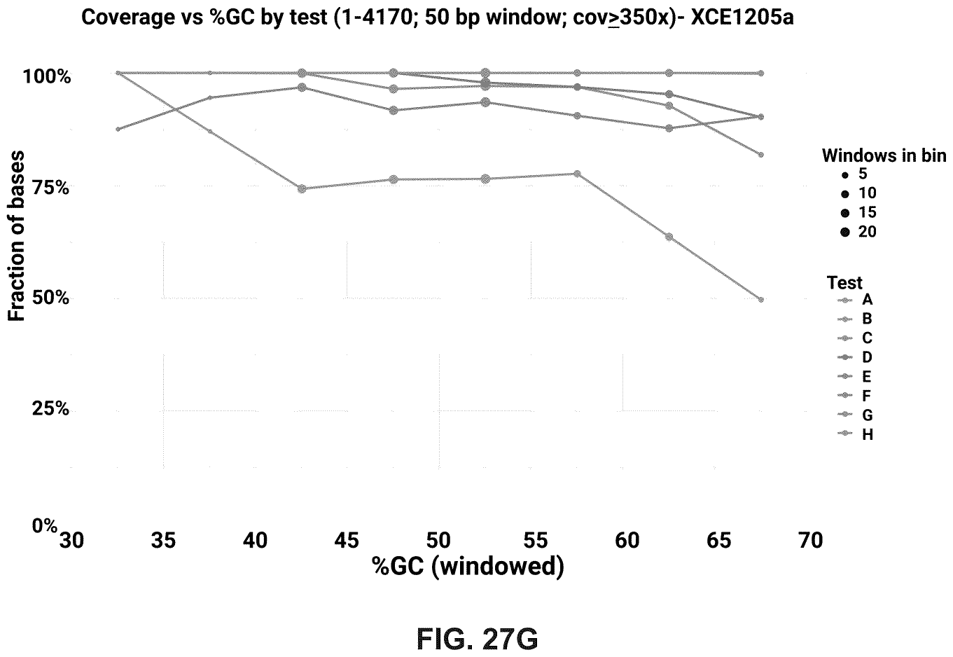

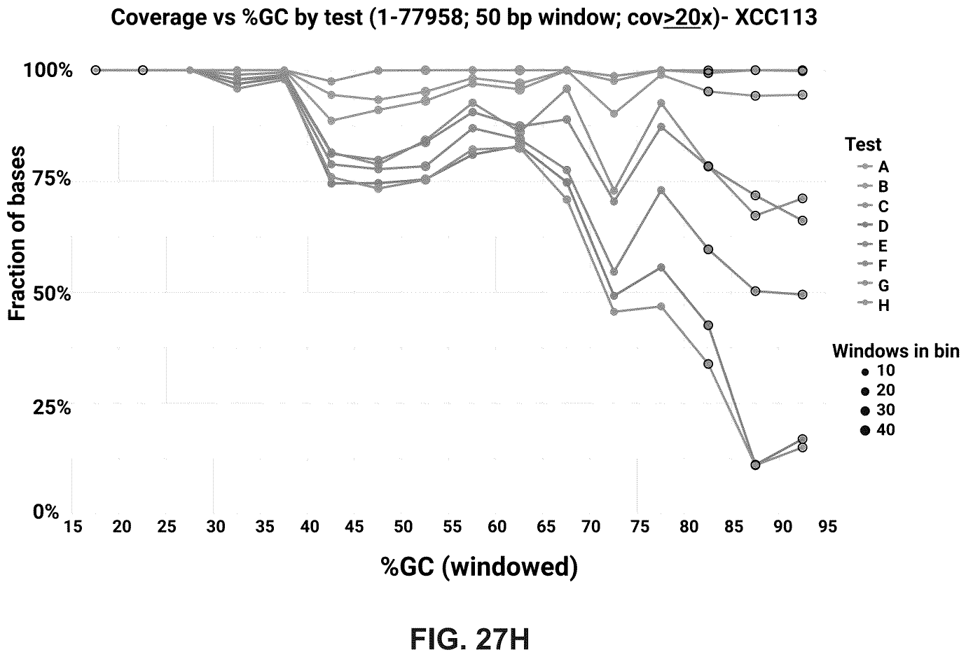

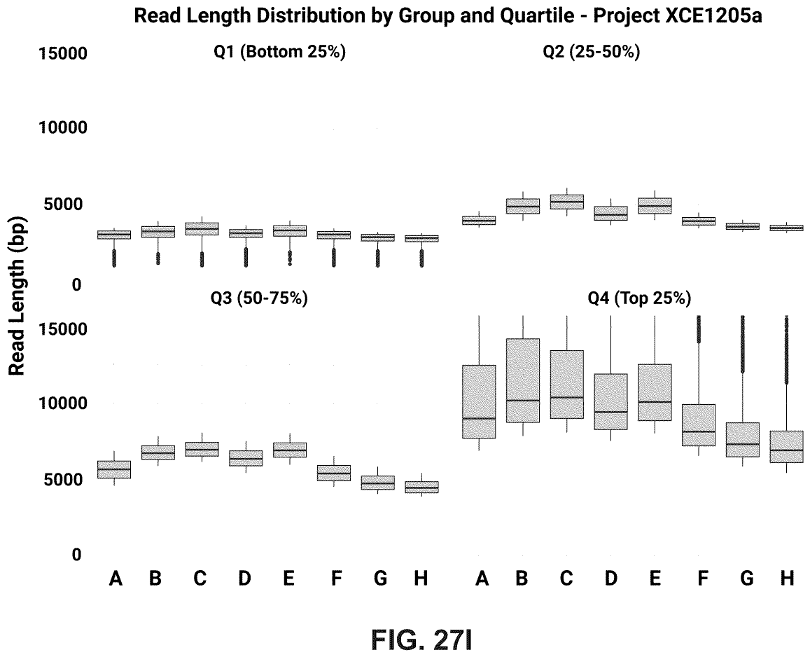

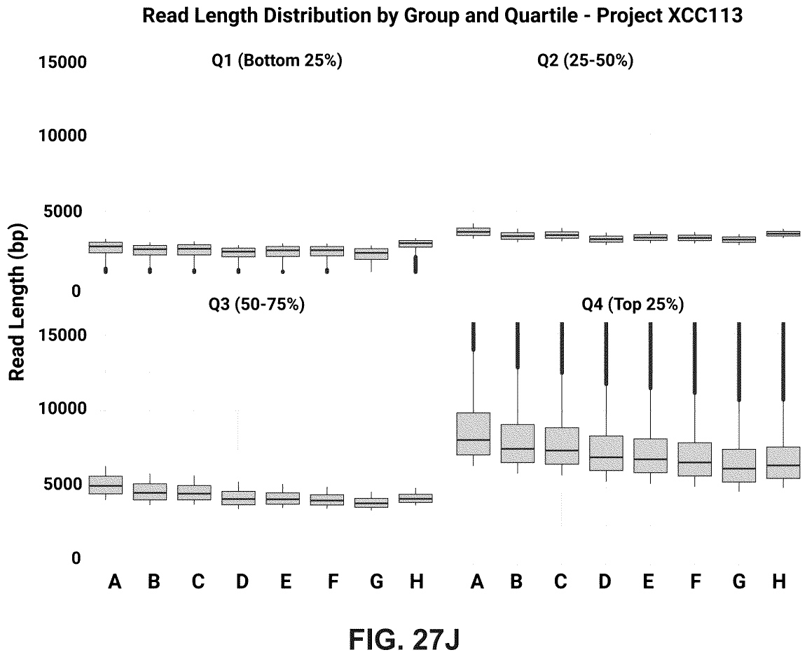

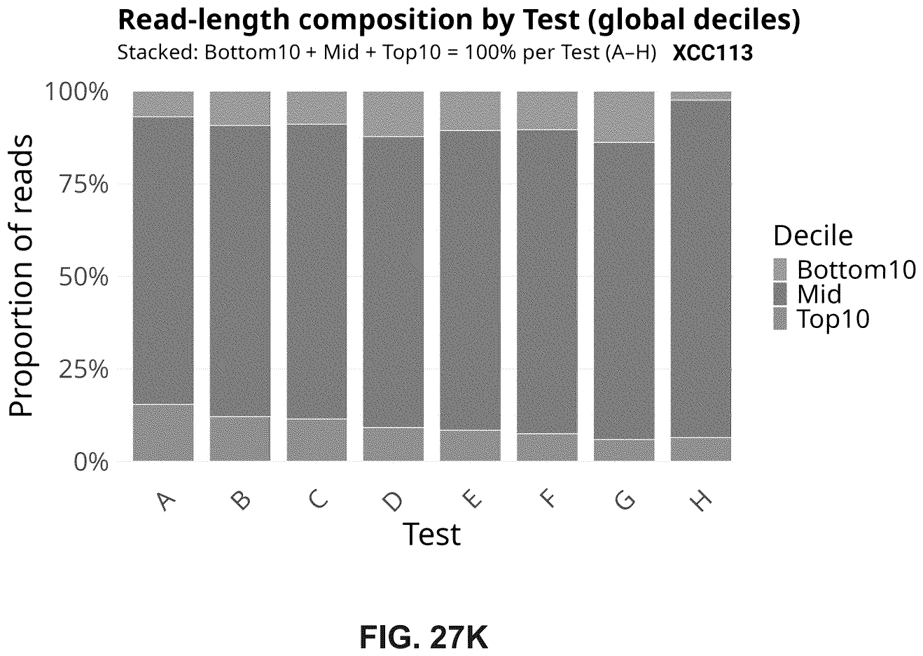

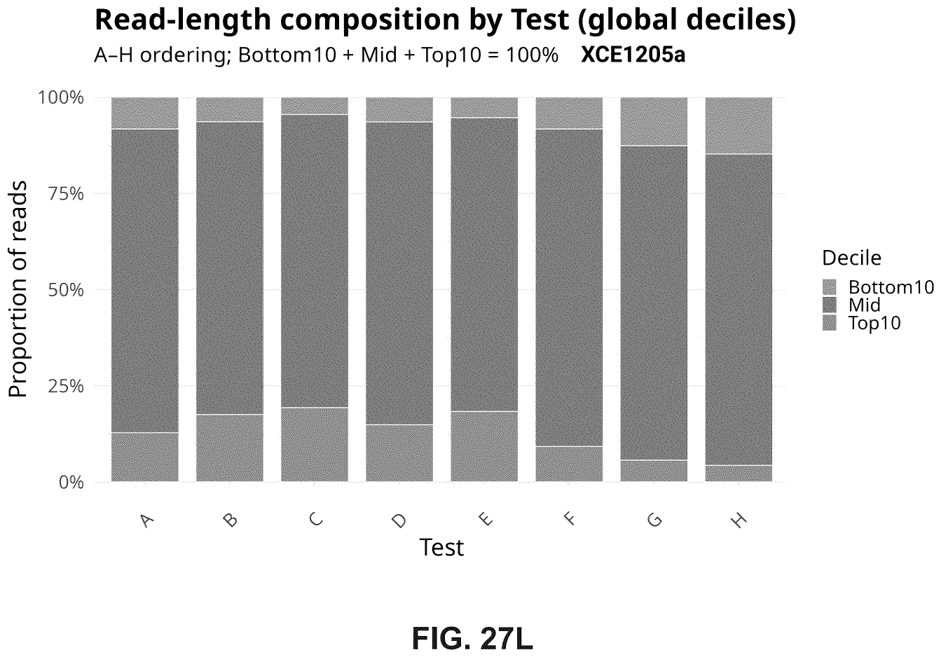

Probe design was further optimized to further improve capture efficiency by testing eight different probe designs in parallel (FIG. 27A). In theory, using probes that hybridize both strands could improve capture by enabling hybridization to both DNA strands with equal efficiency. To test this, a top-stranded probe design was compared to alternating top/bottom and double-stranded probe configurations (Groups A-C, FIG. 27B). All probe sets were 125 nt in length with 20 bp spacing between probes. Unexpectedly, the top-stranded probe design yielded the highest capture efficiency and coverage, outperforming both alternating and double-stranded designs by 1.4- and 3-fold in two different projects, respectively (FIG. 27(C-F)).

Three additional parameters affecting capture efficiency were also examined: (1) increasing the spacing between 125 nt top-stranded probes (Groups D and E, FIG. 27B), (2) extending probe length to 500 nt or 1,000 nt while maintaining 20 bp spacing (Groups F and G, FIG. 27B), and (3) additional biotin moieties (Group H, FIG. 27B). Increasing the probe spacing to 500 bp or 1,000 bp decreased performance, reducing capture efficiency by over twofold. Longer probes produced mixed results-decreasing capture for one target while improving it for another. However, increasing the biotin marks along the probe markedly improve capture efficiency for one target (6.7×) while modestly improving efficiency for the other (FIGS. 27(C and D)). Notably, probes with additional biotin moieties outperformed other designs in achieving improved coverage across GC-rich regions (FIG. 27(E-H)). The overall length distribution of mapped reads was minimally affected by the parameters tested (FIGS. 27(I and J)). However, when examining the longest top 10% of reads across conditions, probes more heavily modified with biotin skewed toward shorter reads (FIGS. 27(K and L)). The results support that the 125-nt probes at 20 bp spacing on the same strand with additional biotin moieties result in the best overall capture and coverage. For particularly GC-rich targets (>70%, >50 bp stretches), moderate addition of biotin during probe synthesis can improve coverage with the caveat of a slight shift towards shorter reads. The improved capture efficiency and coverage by using optimized probe design will allow higher levels of multiplexity and further reduce costs.

### Discussion

Multi-kilobase KIs are critical for the generation of various types of mouse and cell line models, such as reporter lines, humanization, gene replacement, inducible overexpression and conditional KI. The use of rAAV donors for editing single cell embryos eliminates the need for microinjection when coupled with RNP electroporation. As importantly, both birth rate and founder rate are greatly improved. The caveat is the size limit of cargo to 4.7 kb including homology arms and ITRs, leaving maximum insert to be below 4 kb, which as demonstrated herein, can be overcome by consecutive insertions of two or three rAAV donors.

>100 mouse models were generated using 1-, 2- or 3-rAAV donors in one step to deliver inserts up to over 6.6 kb in size. The most significant correlation is between KI efficiency and insert/homology length with one rAAV (FIG. 2D). Previous work using plasmid donors supports a positive correlation between length of homology arms and HDR, with efficiency plateaus achieved at 1-2 kb, but the most marked effect is the poor performance of shorter arms (100-500 bp) that are <0.5 in insert/total homology, lowering HDR rates by half or more when compared to longer arms. The homology arm length requirement further limits the insert size to around 3 kb. When more than one rAAV donor is used, donor 1 and/or donor 2 each bring in an additional gRNA site for recutting upon integration (FIG. 3A). Efficiency was related to insert size, with efficiency decreasing as insert size increased. Nonetheless, most projects only required two mouse sessions to complete (˜200-300 embryos).

Even though overall excellent efficiencies were achieved, it is not failproof with two sessions to obtain founders for a given model. Sufficient homology length to insert size correlates positively to better efficiency. Additional contributing variables can include purity and accuracy of titer of AAV preps, gRNA cutting efficiency, sequence context at target site, variation in manipulation of the embryos during a given session, etc., or any combinations of the above. For example, among the failed projects using one or two donors, each had a project that the donor differed only by a point mutation from the one that worked well, likely because of either rAAV quality/titer and/or arbitrary issues during the mouse session. Additionally, almost all multi-donor projects target the ROSA26 locus with very similar lengths of homology arms (˜800 bp each), yet the founder rate varies from 0-70%. Specifically, two of eight projects shared the same donor 1 and homology arms for donor 2 failed, yet the other six had founder rates ranging from 7.5% to 20%. Using multiple rAAV donors, titering by qPCR does not always reflect the actual viral concentration that can efficiently enter the nucleus to act as repair templates. Given the sequential nature of multiple insertion events, relative titer accuracy may be a leading contributing factor to variance in targeting efficiency.

The same rAAV strategy worked in iPSCs and cancer cell lines. Even though plasmid donors can be nucleofected, and editing is achievable, low survival post nucleofection and low targeting efficiency were often observed. In contrast, project XCB910b used a single rAAV to insert a GFP tag at the AAVS1 site, and showed a 10-fold increase in targeting efficiency compared to plasmid donor (FIG. 22(A-B)).

As importantly, in cells like BV2 and T cells, where exogenous DNA is cytotoxic, rAAV donors make a drastic difference. Using three rAAV donors, BV2 can be edited to have 6.7 kb large insertion (FIGS. 7(A-B) and 23(A-C)). And XCD47c, using two rAAV donors, reached about 14% positive clones in HEK293 cells and iPSCs (FIGS. 24(A-B) and 25(A-D)), compared to using a plasmid donor at less than 1%. Sequential insertion with AAV requires smaller plasmid constructs that are generally easier to synthesize than a single plasmid donor. Intermolecular recombination between monomeric circularized rAAV genomes have been reported as intermediate products that contribute to episomal persistence in muscle tissue, and rAAV genome concatenation is hypothesized to precede integration into the host genome. Homology between rAAV genomes is suggested to mediate intermolecular concatenation via base pairing between ITRs, which account for the head-to-tail configuration of episomal rAAV genomes reported in liver and skeletal muscle. Interestingly, when the second gRNA was left out in nucleofection of XCD47c, positive junctions were obtained in three out of 72 clones in HEK293 cells, compared to 10 out 75 with both gRNAs, implying the possibility of recombination between rAAV donors happens before HDR at the endogenous locus. The same targeting in iPSCs produced junction positive clones with both gRNAs, and without the second gRNA. Recent findings show that rAAV genome concatenation can bridge cis-acting transcriptional modifiers to rAAV expression cassettes delivered in trans, with nuclear concatemer formation detectable as early as 12 hours post-transduction. These concatamers may undergo further processing, providing a template for precise knock-in at a lower efficiency.

Genotyping becomes increasingly challenging with increasing insert size by junction PCRs, using one primer annealing to the target locus outside of a homology arm and the other in the insert to ensure site-specific insertion. Overlapping junction amplicons are more diagnostic but quickly become several kb in length with large inserts. Furthermore, multiple pairs of junction PCRs are necessary with sequential insertions for genotyping (FIG. 12(A-G)) and to confirm the final insert, yet some target sites and/or inserts are difficult to PCR amplify, and positive junctions do not guarantee the two junctions were amplified from the same allele, i.e. partial insertions can lead to false positives. Additionally, CRISPR-mediated targeted integration processes can introduce undesired by-products, such as random integration of donor templates, homology-independent insertion in full or partially into off-target cleavage sites, on-target large deletions and potentially chromosomal translocations, none of which can be detected by junction PCRs. Genotyping can be a time and resource-consuming obstacle for establishing sophisticated research models and potentially skewing data obtained if genotyping is inaccurate.

Cost-effective, high-throughput LOCK-seq was developed to not only screen for accurate KI allele but also detect large indels and donor random integration, providing comprehensive characterization of the genome. By using less than 1 μg of genomic DNA per sample, clonal cell populations and founder animals with different edits can be multiplexed for screening. This can be achieved with biotinylated oligo probes or probes synthesized via a two-step PCR process and loaded into the same MinION flow cell, which typically produces 20-30 Gb of total output and up to thousands of reads per sample. Although some regions may have lower sequencing depth due to secondary structure, sequence context, and probe performance, the method provides reliable and fast results with an average turnaround of under a week. This enables quick identification of knock-in clones and founder animals, thus minimizing culture time and animal husbandry costs.

LOCK-seq significantly reduces per sample cost compared to nCATS and related methods for three main reasons. First, LOCK-seq libraries result in more productive sequencing events on the MinION than those of nCATS. nCATS is amplification-free and relies on CRISPR/Cas9 to bind and expose the DNA ends of select loci for ligation of the motor protein required for ONT sequencing. It is hypothesized that even though the majority of DNA loaded on the flow cell is not competent for sequencing, it can transiently interact with pores or adapter ligated molecules, interfering with sequencing. On the other hand, LOCK-seq achieves greater than 10-fold increase in capture efficiency via hybridization-mediated target enrichment and higher output per flow cell with significantly more efficient ligation between the PCR amplicons of enriched sequences and the motor protein to result in greater than 100 folder increase in coverage than nCATS. Second, LOCK-seq enables low cost and flexible probe generation. Custom synthetic oligo probes labeled with 5′ end biotin were first used, which is pricy. It was then shown that probes generated by asymmetric PCR are versatile and efficient. Panels of 5-10 asymmetric PCR probes (<500 bp in length, 5′ biotinylated, spaced ˜20 bp apart along the flanking genomic regions, homology arms and the insert) can be produced in under an hour. Each set of asymmetric PCRs generates enough probes for hundreds of hybridizations. In contrast, nCATS and related methods require one or more gRNAs that are specific to the target (Table 1). To further improve capture efficiency, we tested different probe length, density, strand distribution and with additional biotin-modified bases incorporated (FIG. 21(A-B)). The data shows that probes of 125 nt, with 20 bp spacing on the same strand with additional biotin-modified internal bases resulted in the highest capture efficiency with minimal increase in probe costs. The preference for probes designed against the same strand may imply the impact of probe density, whereas more than one biotin/streptavidin interaction/probe seems to be needed for maximal capture of multi-kilobase DNA molecules. There is still room to further optimize probe design and production. Finally, fragmentation and barcoding are straightforward. Using Tn5 or Covaris g-TUBEs for fragmentation, barcodes can be ligated to the DNA to facilitate multiplexed target capture. Tn5 fragmentation typically yields post-hybridization reads of 1-2 kb, while Covaris g-TUBEs yield 4-6 kb-long reads, sufficient to obtain single contiguous reads with coverage across the entire transgene and its flanking genomic sequences. In cases where read lengths were shorter than the transgene size, assembly tools like Canu can be used to generate a high-quality consensus sequence, proving the presence of an insertion event, or lack thereof. Both Tn5 tagmentation and the Covaris g-TUBE fragmentation work for LOCK-seq. The latter only requires a one-minute centrifugation and yields longer fragment lengths, averaging around 10 kb. Tn5 tagmentation has higher throughput, is more suitable for an initial screen and can use cell lysates generated with Quick Extract instead of column-purified genomic DNA preparations (FIG. 26(A-D)). It was found that it is critical to use low cycle numbers for both PCR steps to achieve longer read length, 6-8 cycles for barcoding, and 16-18 cycles for post-hybridization PCR.

In addition to on-target insertion events, which can be precise or with imperfections (usually various sized deletions in the insert), LOCK-seq was used to identify concatenation of donors at the target site and random integrations of donors as well as genomic locations of the randomly inserted donors (FIG. 6(A-E)). Both random integration and concatenation of the donors potentially skew phenotypes of the cell or mouse models. Using LOCK-seq to screen and choose correct clones and founders are critical to the precision of the research models. LOCK-seq is agnostic to the format of donors used, and equal success has been achieved at genotyping models generated with plasmid donors. Further, the method should be readily adaptable to be used for genomic localization of transgenes created by various means, such as via lentiviral integration and transposition, and for detection and confirmation of genomic rearrangement events. The upper limit of insert size for reliable detection is currently at 10-20 kb.

The presence of artifacts due to fusion reads was explored and found to be <2%. The difference between artifacts and real fusion events is that real fusions have a distinct junction breakpoint that is supported by multiple independent reads, as opposed to artifacts which lack a consistent fusion junction breakpoint. When mapping to the whole genome, split reads will inevitably arise at low complexity sites, so unique sequence elements were used as anchors when mapping, ensuring that multiple independent reads traverse these regions. This increases confidence when curating the data. Critically, PCR is followed-up with to confirm putative off-target sites identified from LOCK-seq are not sequencing artifacts.

Nucleases potentially have off-target activities, which are not fully understood or well predicted, possibly leading to additional insertion of the donor template, with or without relying on homology. Using LOCK-seq, recurring random insertion sites among different founders and clones are likely to be off target sites, instead of truly “random”. So far, potential off-targeting cleavage-mediated insertion sites have not been detected in either mice or cell lines.

It has been demonstrated that LOCK-seq works well to characterize the post-edit genome. Yet several limitations of LOCK-seq exist. First, the need for two rounds of low-cycle PCRs restricts read length (FIG. 14A) and renders LOCK-seq to be more susceptible to coverage dropouts in GC-rich regions, which can be partially overcome by incorporation of more biotinylated bases in the probe (FIG. 27(A-L)). nCATS, on the other hand, is PCR-free, has longer read length and preserves methylation readout. With the current conditions described herein, read lengths of 4-6 kb on average were obtained, which limits the chance to obtain individual reads spanning from the genomic region outside one homology arm, across large inserts, especially with concatemers, to the genomic region outside of the other homology arm. Optimization of fragmentation methods, PCR conditions and probe design can yield and enrich longer fragments more efficiently. Additionally, it is difficult to apply this method to a transfected pool shortly after transfection, given the abundance of rAAV genome or plasmid donor, compared to the copy number of genomic targets. Finally, analysis of undesired events requires manual curation. A more automated pipeline is currently being developed to facilitate analysis. Even though the advantages of LOCK-seq compensate for these limitations enough to make it a powerful method, further improvements can enable LOCK-seq to be even more valuable.

In conclusion, it is shown that large, multi-kilobase KIs can be achieved efficiently in mouse embryos and cell lines via HDR using more than one rAAV donor and CRISPR/Cas9, and LOCK-seq reliably verifies on-target, precise KIs and identifies undesired partial Kis, donor concatenation and random integrations, enabling accurate selection of founders and clones to ensure model precision. Finally, LOCK-seq can also be used for genomic localization of transgenes created by various means, such as via lentiviral integration and transposition, and for detection and confirmation of genomic rearrangement events.

What is claimed is:

1. A method for preparing long-read sequence samples for multiplexing, the method comprising:
providing a genomic DNA sample;
barcoding the genomic DNA sample with an adapted Tn5 barcode to generate a barcoded material;
fragmenting the barcoded material, wherein the fragmented barcoded material is longer than about 2 kb; and
amplifying the fragmented barcoded material prior to multiplexing.

2. The method of claim 1, wherein the genomic DNA sample is a low-input genomic DNA sample.

3. The method of claim 2, wherein the low-input genomic DNA sample comprises at least about 250 ng and not more than about 5 μg.

4. The method of claim 1, wherein the adapted Tn5 barcode is pre-loaded with unique universal flanking sequences.

5. The method of claim 4, wherein the unique universal flanking sequences comprise Illumina P5 and P7.

6. The method of claim 1, wherein the fragmenting is performed at a temperature of about 37° C.

7. The method of claim 6, wherein the fragmenting is performed with a buffer comprising PEG, TAPS, and MgCl₂.

8. The method of claim 7, wherein the fragmenting comprises an incubation time of at least about 15 minutes and not more than about 20 minutes.

9. The method of claim 1, wherein the barcoding and fragmenting steps are performed simultaneously in a tagmentation step.

10. The method of claim 1, wherein the amplifying is performed in two rounds of PCR.

11. A method for generating a knockin (KI) model, the method comprising:
providing at least two rAAV donor vectors; and
inserting the more than one rAAV donor vectors with CRISPR/Cas9 to generate the KI model.

12. The method of claim 11, wherein the KI model is a multi-kilobase KI model.

13. The method of claim 11, wherein providing at least two rAAV donor vectors comprises providing at least three rAAV donor vectors.

14. The method of claim 11, wherein the KI model is a mouse embryo KI model.

15. The method of claim 11, wherein the KI model is an iPSC cell line KI model.

16. A knockin (KI) model, wherein the KI model was synthesized according to the method comprising:
providing at least two rAAV donor vectors; and
inserting the more than one rAAV donor vectors with CRISPR/Cas9 to generate the KI model.

17. The KI model of claim 16, wherein the KI model is a multi-kilobase KI model.

18. The KI model of claim 16, wherein providing at least two rAAV donor vectors comprises providing at least three rAAV donor vectors.

19. The KI model of claim 16, wherein the KI model is a mouse embryo KI model.

20. The KI model of claim 16, wherein the KI model is an iPSC cell line KI model.
